SEQUENCE LISTING (97 sequences)

SEQ ID NO: 1 - protein, 157 aa, synthetic construct
DLGKKLLQAARAGQLDEVRELLKAGADVNAKDWSGFTPLHIAAVQGHLEIVEVLLKAGADVNAKDHSGWTPLHVAAFSGHLEIVEVLLKAGADVNAKDATGQTPLHLAARTGHLEIVEVLLKAGADVNAQDKSGKTPADLAARAGHQDIAEVLQKAA

SEQ ID NO: 2 - protein, 124 aa, synthetic construct
DLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 3 - protein, 157 aa, synthetic construct
DLGKKLLQAARAGQLDEVRELLKAGADVNAKDEQGRTPLHIAAASGHLEIVEVLLKAGADVNAKDWFGITPLHIAAWWGHLEIVEVLLKAGADVNAKDQEGRTPLHIAAAIGHLEIVEVLLKAGADVNAQDKSGKTPADLAARAGHQDIAEVLQKAA

SEQ ID NO: 4 - protein, 124 aa, synthetic construct
DLGEKLLLAAALGQDDEVRELLKAGADVNAKDVQGSTPLHLAAVEGHLEIVEVLLKAGADVNAKDQWGQTPLHVAAIWGHLEIVEVLLKAGADVNAQDELGKTPADLVALQGHEDIAEVLQKAA

SEQ ID NO: 5 - protein, 124 aa, synthetic construct
DLGAKLLRAAKKGQDDEVRELLKAGADVNAKDELGFTPLHVAAEEGHLEIVEVLLKAGADVNAKDSWGATPLHVAAWVGHLEIVEVLLKAGADVNAQDQEGQTPADLAAEAGHEDIAEVLQKAA

SEQ ID NO: 6 - protein, 124 aa, synthetic construct
DLGKKLLEAAEEGQDDEVRELLKAGADVNAKDEWGSTPLHIAAWVGHLEIVEVLLKAGADVNAKDSLGATPLHLAAEAGHLEIVEVLLKAGADVNAQDLKGKTPADLAALEGHEDIAEVLQKAA

SEQ ID NO: 7 - protein, 124 aa, synthetic construct
DLGQKLLRAAKKGQDDEVRELLKAGADVNAKDEQGWTPLHNAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 8 - protein, 124 aa, synthetic construct
DLGKKLLEAARAGQDDEVRELLKAGADVNAKDEYGLTPLYLATAHGHLEIVEVLLKAGADVNAVDAIGFTPLHLAAFIGHLEIAEVLLKAGADVNAQDKFGKTPADIAAGAGNEDIAEVLQKAA

SEQ ID NO: 9 - protein, 124 aa, synthetic construct
DLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAA

SEQ ID NO: 10 - protein, 272 aa, synthetic construct
DLGKKLLEAARAGQDDEVRELLKAGADVNAKDEYGLTPLYLATAHGHLEIVEVLLKAGADVNAVDAIGFTPLHLAAFIGHLEIAEVLLKAGADVNAQDKFGKTPADIAAGAGNEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 11 - protein, 272 aa, synthetic construct
DLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 12 - protein, 272 aa, synthetic construct
DLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAAGSPTPTPTTPTPTPTTPTPTPTGSDLGKKLLEAARAGQDDEVRELLKAGADVNAKDEYGLTPLYLATAHGHLEIVEVLLKAGADVNAVDAIGFTPLHLAAFIGHLEIAEVLLKAGADVNAQDKFGKTPADIAAGAGNEDIAEVLQKAA

SEQ ID NO: 13 - protein, 272 aa, synthetic construct
DLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAAGSPTPTPTTPTPTPTTPTPTPTGSDLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAA

SEQ ID NO: 14 - protein, 305 aa, synthetic construct
DLGKKLLEAARAGQDDEVRELLKAGADVNAKDEYGLTPLYLATAHGHLEIVEVLLKAGADVNAVDAIGFTPLHLAAFIGHLEIAEVLLKAGADVNAQDKFGKTPADIAAGAGNEDIAEVLQKAAGSPTPTPTTPTPTPTTPTPTPTGSDLGKKLLQAARAGQLDEVRELLKAGADVNAKDEQGRTPLHIAAASGHLEIVEVLLKAGADVNAKDWFGITPLHIAAWWGHLEIVEVLLKAGADVNAKDQEGRTPLHIAAAIGHLEIVEVLLKAGADVNAQDKSGKTPADLAARAGHQDIAEVLQKAA

SEQ ID NO: 15 - protein, 305 aa, synthetic construct
DLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAAGSPTPTPTTPTPTPTTPTPTPTGSDLGKKLLQAARAGQLDEVRELLKAGADVNAKDEQGRTPLHIAAASGHLEIVEVLLKAGADVNAKDWFGITPLHIAAWWGHLEIVEVLLKAGADVNAKDQEGRTPLHIAAAIGHLEIVEVLLKAGADVNAQDKSGKTPADLAARAGHQDIAEVLQKAA

SEQ ID NO: 16 - protein, 305 aa, synthetic construct
DLGKKLLQAARAGQLDEVRELLKAGADVNAKDEQGRTPLHIAAASGHLEIVEVLLKAGADVNAKDWFGITPLHIAAWWGHLEIVEVLLKAGADVNAKDQEGRTPLHIAAAIGHLEIVEVLLKAGADVNAQDKSGKTPADLAARAGHQDIAEVLQKAAGSPTPTPTTPTPTPTTPTPTPTGSDLGKKLLEAARAGQDDEVRELLKAGADVNAKDEYGLTPLYLATAHGHLEIVEVLLKAGADVNAVDAIGFTPLHLAAFIGHLEIAEVLLKAGADVNAQDKFGKTPADIAAGAGNEDIAEVLQKAA

SEQ ID NO: 17 - protein, 305 aa, synthetic construct
DLGKKLLQAARAGQLDEVRELLKAGADVNAKDEQGRTPLHIAAASGHLEIVEVLLKAGADVNAKDWFGITPLHIAAWWGHLEIVEVLLKAGADVNAKDQEGRTPLHIAAAIGHLEIVEVLLKAGADVNAQDKSGKTPADLAARAGHQDIAEVLQKAAGSPTPTPTTPTPTPTTPTPTPTGSDLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAA

SEQ ID NO: 18 - protein, 305 aa, synthetic construct
DLGKKLLQAARAGQLDEVRELLKAGADVNAKDWSGFTPLHIAAVQGHLEIVEVLLKAGADVNAKDHSGWTPLHVAAFSGHLEIVEVLLKAGADVNAKDATGQTPLHLAARTGHLEIVEVLLKAGADVNAQDKSGKTPADLAARAGHQDIAEVLQKAAGSPTPTPTTPTPTPTTPTPTPTGSDLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAA

SEQ ID NO: 19 - protein, 272 aa, synthetic construct
DLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAAGSPTPTPTTPTPTPTTPTPTPTGSDLGEKLLLAAALGQDDEVRELLKAGADVNAKDVQGSTPLHLAAVEGHLEIVEVLLKAGADVNAKDQWGQTPLHVAAIWGHLEIVEVLLKAGADVNAQDELGKTPADLVALQGHEDIAEVLQKAA

SEQ ID NO: 20 - protein, 272 aa, synthetic construct
DLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAAGSPTPTPTTPTPTPTTPTPTPTGSDLGAKLLRAAKKGQDDEVRELLKAGADVNAKDELGFTPLHVAAEEGHLEIVEVLLKAGADVNAKDSWGATPLHVAAWVGHLEIVEVLLKAGADVNAQDQEGQTPADLAAEAGHEDIAEVLQKAA

SEQ ID NO: 21 - protein, 272 aa, synthetic construct
DLGIKLLFAAAKGQDDEVRELLKAGADVNAKDFQGVTPLHIAAQSGHLEIVEVLLKAGADVNAKDVTGDTPLHLAAQHGHLEIVEVLLKAGADVNAQDERGWTPADLAADWGHEDIAEVLQKAAGSPTPTPTTPTPTPTTPTPTPTGSDLGKKLLEAAEEGQDDEVRELLKAGADVNAKDEWGSTPLHIAAWVGHLEIVEVLLKAGADVNAKDSLGATPLHLAAEAGHLEIVEVLLKAGADVNAQDLKGKTPADLAALEGHEDIAEVLQKAA

SEQ ID NO: 22 - protein, 272 aa, synthetic construct
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 23 - protein, 305 aa, synthetic construct
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGKKLLQAARAGQLDEVRELLKAGADVNAKDEQGRTPLHIAAASGHLEIVEVLLKAGADVNAKDWFGITPLHIAAWWGHLEIVEVLLKAGADVNAKDQEGRTPLHIAAAIGHLEIVEVLLKAGADVNAQDKSGKTPADLAARAGHQDIAEVLQKAA

SEQ ID NO: 24 - protein, 124 aa, synthetic construct
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 25 - protein, 124 aa, synthetic construct
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 26 - protein, 124 aa, synthetic construct
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 27 - protein, 33 aa, unidentified
KDWSGFTPLHIAAVQGHLEIVEVLLKAGADVNA

SEQ ID NO: 28 - protein, 33 aa, synthetic construct
KDHSGWTPLHVAAFSGHLEIVEVLLKAGADVNA

SEQ ID NO: 29 - protein, 33 aa, synthetic construct
KDATGQTPLHLAARTGHLEIVEVLLKAGADVNA

SEQ ID NO: 30 - protein, 33 aa, synthetic construct
KDEQGWTPLHIAAWKGHLEIVEVLLKAGADVNA

SEQ ID NO: 31 - protein, 33 aa, synthetic construct
KDVFDWTPLHVAAHFGHLEIVEVLLKAGADVNA

SEQ ID NO: 32 - protein, 33 aa, synthetic construct
KDEQGRTPLHIAAASGHLEIVEVLLKAGADVNA

SEQ ID NO: 33 - protein, 33 aa, synthetic construct
KDWFGITPLHIAAWWGHLEIVEVLLKAGADVNA

SEQ ID NO: 34 - protein, 33 aa, synthetic construct
KDQEGRTPLHIAAAIGHLEIVEVLLKAGADVNA

SEQ ID NO: 35 - protein, 33 aa, synthetic construct
KDVQGSTPLHLAAVEGHLEIVEVLLKAGADVNA

SEQ ID NO: 36 - protein, 33 aa, synthetic construct
KDQWGQTPLHVAAIWGHLEIVEVLLKAGADVNA

SEQ ID NO: 37 - protein, 33 aa, synthetic construct
KDELGFTPLHVAAEEGHLEIVEVLLKAGADVNA

SEQ ID NO: 38 - protein, 33 aa, synthetic construct
KDSWGATPLHVAAWVGHLEIVEVLLKAGADVNA

SEQ ID NO: 39 - protein, 33 aa, synthetic construct
KDEWGSTPLHIAAWVGHLEIVEVLLKAGADVNA

SEQ ID NO: 40 - protein, 33 aa, synthetic construct
KDSLGATPLHLAAEAGHLEIVEVLLKAGADVNA

SEQ ID NO: 41 - protein, 33 aa, synthetic construct
KDEQGWTPLHNAAWKGHLEIVEVLLKAGADVNA

SEQ ID NO: 42 - protein, 30 aa, synthetic construct
DLGKKLLQAARAGQLDEVRELLKAGADVNA

SEQ ID NO: 43 - protein, 30 aa, synthetic construct
DLGQKLLRAAKKGQDDEVRELLKAGADVNA

SEQ ID NO: 44 - protein, 30 aa, synthetic construct
DLGEKLLLAAALGQDDEVRELLKAGADVNA

SEQ ID NO: 45 - protein, 30 aa, synthetic construct
DLGAKLLRAAKKGQDDEVRELLKAGADVNA

SEQ ID NO: 46 - protein, 30 aa, synthetic construct
DLGKKLLEAAEEGQDDEVRELLKAGADVNA

SEQ ID NO: 47 - protein, 28 aa, synthetic construct
QDKSGKTPADLAARAGHQDIAEVLQKAA

SEQ ID NO: 48 - protein, 28 aa, synthetic construct
QDRAGETPADLAAQAGHEDIAEVLQKAA

SEQ ID NO: 49 - protein, 28 aa, synthetic construct
QDELGKTPADLVALQGHEDIAEVLQKAA

SEQ ID NO: 50 - protein, 28 aa, synthetic construct
QDQEGQTPADLAAEAGHEDIAEVLQKAA

SEQ ID NO: 51 - protein, 28 aa, synthetic construct
QDLKGKTPADLAALEGHEDIAEVLQKAA

SEQ ID NO: 52 - protein, 33 aa, synthetic construct
  VARIANT 1..33: note = wherein X is selected from any amino acid
KDXXGXTPLHXAAXXGHLEIVEVLLKAGADVNA

SEQ ID NO: 53 - protein, 33 aa, synthetic construct
  VARIANT 1..33: note = wherein X is selected from any amino acid
KDXXGXTPLHXAAXXGHPEIVEVLLKAGADVNA

SEQ ID NO: 54 - protein, 33 aa, synthetic construct
  VARIANT 1..33: note = wherein X is selected from any amino acid
XDXXGXTPLHXAAXXGHLEIVEVLLKXGADVNA

SEQ ID NO: 55 - protein, 33 aa, synthetic construct
  VARIANT 1..33: note = wherein X is selected from any amino acid
XDXXGXTPLHXAAXXGHPEIVEVLLKXGADVNA

SEQ ID NO: 56 - protein, 33 aa, synthetic construct
  VARIANT 1..33: note = wherein X is selected from any amino acid
XDXXGXTPLHXAAXXGHLEIVEVLLKAGADVNA

SEQ ID NO: 57 - protein, 33 aa, synthetic construct
  VARIANT 1..33: note = wherein X is selected from any amino acid
XDXXDXTPLHXAAXXGHLEIVEVLLKAGADVNA

SEQ ID NO: 58 - protein, 30 aa, synthetic construct
  VARIANT 1..30: note = wherein X is selected from any amino acid
DLGXKLLXAAXXGQDDEVRELLKAGADVNA

SEQ ID NO: 59 - protein, 30 aa, synthetic construct
  VARIANT 1..30: note = wherein X is selected from any amino acid
DLGXKLLXAAXXGQLDEVRELLKAGADVNA

SEQ ID NO: 60 - protein, 30 aa, synthetic construct
  VARIANT 1..30: note = wherein X is selected from any amino acid
DLGXKLLEAAXXGQDDEVRELLKAGADVNA

SEQ ID NO: 61 - protein, 30 aa, synthetic construct
  VARIANT 1..30: note = wherein X is selected from any amino acid
DLGXKLLQAAXXGQLDEVRELLKAGADVNA

SEQ ID NO: 62 - protein, 28 aa, synthetic construct
  VARIANT 1..28: note = wherein X is selected from any amino acid
QDXXGXTPADLAAXXGHEDIAEVLQKAA

SEQ ID NO: 63 - protein, 28 aa, synthetic construct
  VARIANT 1..28: note = wherein X is selected from any amino acid
QDXXGXTPADLAAXXGHQDIAEVLQKAA

SEQ ID NO: 64 - protein, 28 aa, synthetic construct
  VARIANT 1..28: note = wherein X is selected from any amino acid
QDXXGXTPADLVAXXGHEDIAEVLQKAA

SEQ ID NO: 65 - protein, 28 aa, synthetic construct
  VARIANT 1..28: note = wherein X is selected from any amino acid
XDXXGXTPXXXXXDXXXEXXXXXXXXAA

SEQ ID NO: 66 - protein, 28 aa, synthetic construct
  VARIANT 1..28: note = wherein X is selected from any amino acid
XDXXGXTPXXXXXRXXXEXXXXXXXXAA

SEQ ID NO: 67 - protein, 28 aa, synthetic construct
  VARIANT 1..28: note = wherein X is selected from any amino acid
XDXXGXTPADLXAXXGHEDIAEVLQKAA

SEQ ID NO: 68 - protein, 28 aa, synthetic construct
  VARIANT 1..28: note = wherein X is selected from any amino acid
XDXXGXTPADLXAXXGHQDIAEVLQKAA

SEQ ID NO: 69 - protein, 12 aa, synthetic construct
MRGSHHHHHHGS

SEQ ID NO: 70 - protein, 24 aa, synthetic construct
GSPTPTPTTPTPTPTTPTPTPTGS

SEQ ID NO: 71 - protein, 26 aa, synthetic construct
MRGSHHHHHHDYKDDDDKPTPTPTGS

SEQ ID NO: 72 - protein, 210 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGSGGGSHHHHHHHHHH

SEQ ID NO: 73 - protein, 201 aa, synthetic construct
MRTEDLPKAVVFLEPQWYRVLEKDSVTLKCQGAYSPEDNSTQWFHNENLISSQASSYFIDAATVDDSGEYRCQTNLSTLSDPVQLEVHVGWLLLQAPRWVFKEEDPIHLRCHSWKNTALHKVTYLQNGKDRKYFHHNSDFHIPKATLKDSGSYFCRGLVGSKNVSSETVNITITQGLAVSTISGGGSGGGSHHHHHHHHHH

SEQ ID NO: 74 - protein, 202 aa, synthetic construct
GMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISGGGSGGGSHHHHHHHHHH

SEQ ID NO: 75 - protein, 225 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQGLNDIFEAQKIEWHEGGGSGGGSHHHHHHHHHH

SEQ ID NO: 76 - protein, 225 aa, synthetic construct
GMRAEDLPKAVVFLEPQWYRVLEKDRVTLKCQGAYSPEDNSTRWFHNESLISSQTSSYFIAAARVNNSGEYRCQTSLSTLSDPVQLEVHIGWLLLQAPRWVFKEEESIHLRCHSWKNTLLHKVTYLQNGKGRKYFHQNSDFYIPKATLKDSGSYFCRGLIGSKNVSSETVNITITQDLAVSSISSFFPPGYQGGGSGGGSHHHHHHHHHHGLNDIFEAQKIEWHE

SEQ ID NO: 77 - DNA, 471 bp, synthetic construct
  source 1..471: mol_type = other DNA
gacctgggtaaaaaactgctgcaagcagcacgtgcaggtcagctggatgaagttcgtgaactgctgaaagcaggcgccgatgttaatgcaaaagattggtctggcttcaccccgctgcatatcgctgctgtgcaaggtcacctggaaattgttgaagttctgctgaaagccggtgcagatgttaatgcaaaagatcactctggctggaccccgctgcatgtggctgctttctctggtcacctggaaattgttgaagttctgctgaaagccggtgcagatgttaatgcaaaagatgctaccggccaaaccccgctgcatctggctgctagaaccggtcacctggaaattgttgaagttctgctgaaagccggtgcagatgttaacgcacaggataaaagcggtaaaacccctgccgatctggcagctcgcgccggtcatcaagatattgctgaagtgctgcagaaggcagca

SEQ ID NO: 78 - DNA, 372 bp, synthetic construct
  source 1..372: mol_type = other DNA
gacttgggacaaaaattattgcgcgctgcaaaaaaaggccaggacgacgaggtgcgtgagttgcttaaagcaggggccgatgttaatgcaaaagatgaacaagggtggacgccgttgcatattgcagcgtggaaaggtcatttagaaatcgtagaggtgcttcttaaagcgggcgctgacgttaacgcaaaagacgtattcgattggacacctttgcatgtcgctgcgcactttggacacttagagatcgtggaggttttattaaaggctggcgcggatgtaaatgcacaggaccgtgccggagagacaccagcggatttagcagcgcaagcgggacacgaagacatcgccgaggtcctgcagaaggcagcg

SEQ ID NO: 79 - DNA, 471 bp, synthetic construct
  source 1..471: mol_type = other DNA
gacctgggtaaaaaactgctgcaagcagcacgtgcaggtcagctggatgaagttcgtgaactgctgaaagcaggcgccgatgttaatgcaaaagatgaacagggccgtaccccgctgcatatcgctgctgcttccggtcacctggaaattgttgaagttctgctgaaagccggtgcagatgttaatgcaaaagattggttcggcatcaccccgctgcatatcgctgcttggtggggtcacctggaaattgttgaagttctgctgaaagccggtgcagatgttaatgcaaaagatcaggaaggccgtaccccgctgcatatcgctgctgctatcggtcacctggaaattgttgaagttctgctgaaagccggtgcagatgttaacgcacaggataaaagcggtaaaacccctgccgatctggcagctcgcgccggtcatcaagatattgctgaagtgctgcagaaggcagcg

SEQ ID NO: 80 - DNA, 372 bp, synthetic construct
  source 1..372: mol_type = other DNA
gacctgggtgaaaagctgctgctggctgctgctctgggtcaggatgatgaagttcgtgaactgctgaaagcaggcgccgatgttaatgcaaaagatgtgcaaggctctaccccgctgcatctggctgctgtggagggtcacctagaaattgttgaagttctgctgaaagccggtgcagatgttaatgcaaaagatcaatggggccaaaccccgctgcatgtggctgctatctggggtcacctggaaattgttgaagttctgctgaaagccggtgcagatgttaacgcacaggatgaactgggcaagacccctgccgatctggtagctctgcaaggtcatgaagatattgcagaagtgctgcagaaggcagca

SEQ ID NO: 81 - DNA, 372 bp, synthetic construct
  source 1..372: mol_type = other DNA
gacctgggtgctaagctgctgcgtgctgctaagaagggtcaggatgatgaagttcgtgaactgctgaaagcaggcgccgatgttaatgcaaaagatgagctgggcttcaccccgctgcatgtggctgctgaggagggtcacctggaaattgttgaagttctgctgaaagccggtgcagatgttaatgcaaaagattcttggggcgctaccccgctgcatgtggctgcttgggtgggtcacctggaaattgttgaagttctgctgaaagccggtgcagatgttaacgcacaggatcaggaaggccagacccctgccgatctggcagctgaagctggtcatgaagatattgcagaagtgctgcagaaggcagca

SEQ ID NO: 82 - DNA, 372 bp, synthetic construct
  source 1..372: mol_type = other DNA
gacctgggtaagaagctgctggaagctgctgaagaaggtcaggatgatgaagttcgtgaactgctgaaagcaggcgccgatgttaatgcaaaagatgagtggggctctaccccgctgcatatcgctgcttgggtgggtcacctggaaattgttgaagttctgctgaaagccggtgcagatgttaatgcaaaagattctctgggcgctaccccgctgcatctggctgctgaggctggtcacctggaaattgttgaagttctgctgaaagccggtgcagatgttaacgcacaggatctgaagggcaagacccctgccgatctggcagctctggaaggtcatgaagatattgcagaagtgctgcagaaggcagca

SEQ ID NO: 83 - DNA, 378 bp, synthetic construct
  source 1..378: mol_type = other DNA
ggatccgacttgggacaaaaattattgcgcgctgcaaaaaaaggccaggacgacgaggtgcgtgagttgcttaaagcaggggccgatgttaatgcaaaagatgaacaagggtggacgccgttgcataatgcagcgtggaaaggtcatttagaaatcgtagaggtgcttcttaaagcgggcgctgacgttaacgcaaaagacgtattcgattggacacctttgcatgtcgctgcgcactttggacacttagagatcgtggaggttttattaaaggctggcgcggatgtaaatgcacaggaccgtgccggagagacaccagcggatttagcagcgcaagcgggacacgaagacatcgccgaggtcctgcagaaggcagcg

SEQ ID NO: 84 - protein, 124 aa, synthetic construct
DLGQKLLKAAQYGQDDEVRILLAAGADVNAKDWAGKTPLHVAAEAGHLEIVEVLLKAGADVNAKDKAGETPLHLAAEQGHLEIVEVLLKAGADVNAQDEWGQTPADLAAYYGLEDIAEVLQKAA

SEQ ID NO: 85 - protein, 124 aa, synthetic construct
DLGAKLLKAAQSGQDDEVRILLAAGADVNAKDKDGYTPLHLAAREGHLEIVEVLLKAGSDVNAKDYYGETPLHQAAEYGHLEIVEVLLKAGADVNAQDEWGATPADLAAQQGHEDIAEVLQKAA

SEQ ID NO: 86 - protein, 124 aa, synthetic construct
DLGKKLLQAARAGQLDEVRELLKAGADVNAKDVYGLTPLHLAAREGHLEIVEVLLKAGADVNAKDAYGETPLHLAAEAGHLEIVEVLLKAGADVNAKDEWGETPLNVAARAGHQDIAEVLQKAA

SEQ ID NO: 87 - protein, 124 aa, synthetic construct
DLGKKLLKAASWGQDDEVRILLAAGADVNAKDNSGWTPLHAAAWYGHLEIVEVLLKAGADVNAKDKTGLTPLHKAAHVGHLEIVEVLLKAGADVNAQDEWGATPADLAASTGHEDIAEVLQKAA

SEQ ID NO: 88 - protein, 124 aa, synthetic construct
DLGHKLLHAAAWGQDDEVRILLAAGADVNAKDQSGWTPLHVAAWYGHLEIVEVLLKAGADVNAKDLKGETPLHLAALVGHLEIVEVLLKAGADVNAQDIAGDTPADLAAIAGHEDIAEVLQKAA

SEQ ID NO: 89 - protein, 272 aa, synthetic construct
DLGQKLLKAAQYGQDDEVRILLAAGADVNAKDWAGKTPLHVAAEAGHLEIVEVLLKAGADVNAKDKAGETPLHLAAEQGHLEIVEVLLKAGADVNAQDEWGQTPADLAAYYGLEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 90 - protein, 272 aa, synthetic construct
DLGAKLLKAAQSGQDDEVRILLAAGADVNAKDKDGYTPLHLAAREGHLEIVEVLLKAGSDVNAKDYYGETPLHQAAEYGHLEIVEVLLKAGADVNAQDEWGATPADLAAQQGHEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 91 - protein, 272 aa, synthetic construct
DLGKKLLQAARAGQLDEVRELLKAGADVNAKDVYGLTPLHLAAREGHLEIVEVLLKAGADVNAKDAYGETPLHLAAEAGHLEIVEVLLKAGADVNAKDEWGETPLNVAARAGHQDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 92 - protein, 272 aa, synthetic construct
DLGKKLLKAASWGQDDEVRILLAAGADVNAKDNSGWTPLHAAAWYGHLEIVEVLLKAGADVNAKDKTGLTPLHKAAHVGHLEIVEVLLKAGADVNAQDEWGATPADLAASTGHEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 93 - protein, 272 aa, synthetic construct
DLGHKLLHAAAWGQDDEVRILLAAGADVNAKDQSGWTPLHVAAWYGHLEIVEVLLKAGADVNAKDLKGETPLHLAALVGHLEIVEVLLKAGADVNAQDIAGDTPADLAAIAGHEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 94 - protein, 272 aa, synthetic construct
DLGQKLLKAAQYGQDDEVRILLAAGADVNAKDWAGKTPLHVAAEAGHLEIVEVLLKAGADVNAKDKAGETPLHLAAEQGHLEIVEVLLKAGADVNAQDEWGQTPADLAAYYGLEDIAEVLQKAAGSPTPTPTTPTPTPTTPTPTPTGSDLGQKLLRAAKKGQDDEVRELLKAGADVNAKDEQGWTPLHNAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 95 - protein, 272 aa, synthetic construct
DLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 96 - protein, 272 aa, synthetic construct
DLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAAGSPTPTPTTPTPTPTTPTPTPTGSDLGQKLLRAAKKGQDDEVRELLKAGADVNAKDEQGWTPLHNAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 97 - protein, 33 aa, synthetic construct
KDXXDXTPLHXAAXXGHLEIVEVLLKAGADVNA

(19) United States
(12) Patent Application Publication
(10) Pub. No.: US 2026/0250338 A1
(43) Pub. Date: Aug. 27, 2026

(54) RECOMBINANT CD16A BINDING PROTEINS AND THEIR USE

(71) Applicant: MOLECULAR PARTNERS AG, Zürich-Schlieren (CH)

(72) Inventors: Alexander LINK, Zürich-Schlieren (CH); Valerie Perrine CALABRO, Zürich-Schlieren (CH); Anja Christina SCHLEGEL, Zürich-Schlieren (CH); Natalia VENETZ ARENAS, Zürich-Schlieren (CH)

(21) Appl. No.: 19/489,731

(22) PCT Filed: May 31, 2024
(86) PCT No.: PCT/EP2024/065105

§ 371 Date: Dec. 3, 2025

(30) Foreign Application Priority Data
Jun. 6, 2023 (EP) 23177833.3

(51) Int. Cl. C07K 14/47; A61K 38/00

(52) U.S. Cl. CPC C07K 14/47; A61K 38/00

(57) ABSTRACT

The present invention relates to recombinant binding proteins comprising an ankyrin repeat domain, wherein the ankyrin repeat domain has binding specificity for CD16a. In addition, the invention relates to nucleic acids encoding such recombinant binding proteins, pharmaceutical compositions comprising such proteins or nucleic acids, and the use of such binding proteins, nucleic acids or pharmaceutical compositions in methods for treating or diagnosing diseases, such as cancer in a mammal, including a human.

### FIELD OF THE DISCLOSURE

The present invention relates to recombinant binding proteins comprising a designed ankyrin repeat domain with binding specificity for CD16a, also known as Fc gamma III receptor (FcγRIIIA). In addition, the invention relates to nucleic acids encoding such binding proteins, pharmaceutical compositions comprising such binding proteins or nucleic acids, and the use of such binding proteins, nucleic acids or pharmaceutical compositions in methods of treating diseases, such as cancer, in a mammal, including a human.

### BACKGROUND

CD16 (also known as FcγRIII) is a low affinity receptor for the Fc portion of some IgGs known to be involved in antibody-dependent cellular cytotoxicity (ADCC) and is a well characterized membrane receptor responsible for triggering target cell lysis by Natural Killer (NK) cells (Mandelboim et al., 1999, PNAS 96:5640-5644). Human NK cells comprise approximately 15% of all lymphocytes and are defined phenotypically by their expression of CD56 and lack of expression of CD3 (Robertson and Ritz, 1990, Blood 76:2421-2438). The majority (approximately 90%) of human NK cells express CD56 at low density (CD56^{dim}) and CD16 at a high level (Cooper et al., 2001, Trends Immunol. 22:633-640). Human CD16 exists as two isoforms, CD16a (FcγRIIIA) and CD16b (FcγRIIIB), that share 96% sequence identity in their extracellular immunoglobulin-binding regions (van de Winkel and Capel, 1993, Immunol. Today 14(5):215-221; Robinson et al. 2018, Proc. of the Nat. Acad. of Sc. 115(1): E72-E81).

CD16a (FcγRIIIA) is expressed on macrophages, mast cells, and NK cells as a transmembrane receptor. On NK cells, the alpha chain of CD16a associates with the immunoreceptor tyrosine-based activation motif (ITAM) containing FcεRIy-chain and/or the T-cell receptor (TCR)/CD3 ζ-chain to mediate signalling (Wirthmueller et al., 1992, J. Exp. Med. 175:1381-1390). The interaction of CD16a with different combinations of homo- and hetero-dimers of the γ and ζ chains has been observed in NK cells, offering the possibility of different signalling pathways via variations of the CD16a complex in NK cells (Anderson et al., 1990, PNAS 87(6):2274-2278; Ackerly et al., 1992, Int. J. Cancer Suppl. 7:11-14).

CD16b (FcγRIIIB) is present on polymorphonuclear granulocytes (PMN) as a glycosyl-phosphatidylinositol (GPI)-anchored receptor (FcγRIIIB isoform), which cannot trigger tumour cell killing (van de Winkel and Capel, 1993, supra). In addition, CD16b exists as a soluble receptor in serum, and upon binding to antibodies may trigger side-effects via the formation of immune complexes in vivo.

FcγR-expressing effector cells have been shown to be involved in destroying tumour cells via ADCC. This has led to the development of several immunotherapeutic approaches to cancer therapy, which involve the use of FcγR activities, for example, tumour specific monoclonal antibodies can mediate destruction of tumour cells by ADCC induced via binding to FcγRs, and bi-specific molecules with one specificity for tumour cells and the other specificity for either an FcγRI on granulocytes or FcγRIII on NK cells have also been developed in efforts to improve effector cell recruitment (van Spriel et al., 2000 Immunol. Today, 21:391-397).

NK cells are professional cell killers and, unlike T cells, tend to exist in constitutively activated states not requiring additional (pre-)stimulation. In contrast, resting cytotoxic T-cells require activation via the binding of an antigen-MHC complex and subsequent co-stimulation via CD28. Thus, amongst the immune effector cells, the NK cell is one of the more attractive candidates for use in immunotherapy, and bi-specific antibodies with specificity to CD16 and HER-2/neu or CD16 and CD30 have been developed (Arndt et al., 1999 Blood 94:2562-2568; McCall et al., 2001 J. Immunol. 166:6112-6117).

Only few binding proteins described so far can distinguish between the two CD16 isoforms (CD16a and CD16b), a majority of them being antibodies or fragments thereof. The monoclonal antibody (mAb) 1D3, which only binds to CD16b (i.e. FcγRIIIB) expressed on neutrophils, the monoclonal antibodies which recognise different allotypic forms of CD16b (Ory et al. 1989, J. Clin. Invest. 83:1676-81; Huizinga et al. 1990 Blood 75:213-7; Tamm & Schmidt 1996, J. Immunol. 1996 157:1576-1581), and the anti-CD16a binders described in U.S. Pat. No. 9,035,026B2 are such example of CD16 isoform specific binders.

Thus, there remains a need for new CD16a-specific binding proteins with beneficial properties. Such binding proteins may be useful for therapeutic and diagnostic approaches for the treatment and characterization of diseases, including cancer.

### SUMMARY

The present invention relates to recombinant binding proteins comprising an ankyrin repeat domain, wherein the ankyrin repeat domain has binding specificity for CD16a. In addition, the invention relates to nucleic acids encoding such recombinant binding proteins, pharmaceutical compositions comprising such proteins or nucleic acids, and the use of such binding proteins, nucleic acids, or pharmaceutical compositions in methods for treating or diagnosing diseases, such as cancer, e.g., acute myeloid leukemia (AML), in a mammal, including a human.

Recombinant binding proteins of the invention specifically bind to or target CD16a expressed by immune cells such as NK cells or macrophages. Such binding proteins of the invention can serve as a tool or as a building block for the generation of new therapeutic or diagnostic agents. Also disclosed herein are recombinant binding proteins, in which the CD16a-specific ankyrin repeat domains are combined with one or more other functional moieties in one molecule. Such other functional moieties include a half-life extending moiety and/or a binding moiety, preferably ankyrin repeat domains, with binding specificity for a Disease-Associated Antigen (DAA), such as, e.g., a Tumor Associated Antigen (TAA) or an Infection Associated Antigen (IAA), preferably a Virus Associated Antigen (VAA). As such, recombinant binding proteins of the invention with binding specificity for CD16a are useful for the generation of novel therapeutic molecules, which may provide an improved toxicity profile and/or therapeutic window as compared to current therapeutic modalities.

In one aspect, the invention provides a recombinant binding protein comprising an ankyrin repeat domain having binding specificity for CD16a, wherein said ankyrin repeat domain comprises an ankyrin repeat module having an amino acid sequence selected from the group consisting of (1) SEQ ID NOs: 27 to 41, and (2) sequences in which up to 9 amino acids in any of SEQ ID NOs: 27 to 41 are substituted by other amino acids.

In one aspect, the invention provides a recombinant binding protein comprising an ankyrin repeat domain with binding specificity for CD16a, wherein said ankyrin repeat domain comprises an amino acid sequence with at least 80% amino acid sequence identity with any one of SEQ ID NOs: 1 to 7, and wherein A at the second last position of SEQ ID NOs: 1 to 7 is optionally substituted by L, and/or A at the last position of SEQ ID NOs: 1 to 7 is optionally substituted by N.

In one aspect, the invention provides a recombinant binding protein comprising an ankyrin repeat domain with binding specificity for CD16a, wherein said recombinant binding protein further comprises one, two or three binding moieties, wherein each of said binding moieties has binding specificity for a disease-associated antigen.

In one aspect, the invention provides a recombinant binding protein comprising an ankyrin repeat domain with binding specificity for CD16a as described herein, wherein said recombinant binding protein further comprises a half-life extending moiety.

In one aspect, the invention provides nucleic acids encoding the recombinant binding proteins of the invention and pharmaceutical compositions comprising the recombinant binding protein of the invention or the nucleic acid of the invention and optionally a pharmaceutically acceptable carrier and/or diluent.

In one aspect, the invention provides a method of activating immune cells, the method comprising the step of administering to a subject in need thereof an effective amount of the recombinant binding protein of the invention, the nucleic acid of the invention or the pharmaceutical composition of the invention.

In one aspect, the invention provides the recombinant binding protein of the invention, the nucleic acid of the invention or the pharmaceutical composition of the invention for use in a method of treating a medical condition. In one embodiment, said medical condition is an infectious disease, preferably a viral infectious disease. In a preferred embodiment, said medical condition is a cancer.

In one aspect, the invention provides a method of treating a medical condition in a human subject, the method comprising administering to said subject a therapeutically effective amount of the recombinant binding protein of the invention, the nucleic acid of the invention or the pharmaceutical composition of the invention. In a preferred embodiment, said medical condition is a cancer.

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).
E1. A recombinant binding protein comprising an ankyrin repeat domain having binding specificity for CD16a, wherein said ankyrin repeat domain comprises an ankyrin repeat module having an amino acid sequence selected from the group consisting of (1) SEQ ID NOs: 27 to 41, and (2) sequences in which up to 9 amino acids in any of SEQ ID NOs: 27 to 41 are substituted by other amino acids.
E2. The recombinant binding protein according to E1, wherein said ankyrin repeat module is a first ankyrin repeat module and wherein said ankyrin repeat domain further comprises a second ankyrin repeat module having an amino acid sequence selected from the group consisting of (1) SEQ ID NOs: 27 to 41 and (2) sequences in which up to 9 amino acids in any of SEQ ID NOs: 27 to 41 are substituted by other amino acids.
E3. The recombinant binding protein according to E2, wherein said first and second ankyrin repeat modules each independently comprise an amino acid sequence selected from the group consisting of (1) SEQ ID NO: 30, 31, 35, 36 and 37 to 41 and (2) sequences in which up to 9 amino acids in any of SEQ ID NO: 30, 31, 35, 36 and 37 to 41 are substituted by other amino acids.
E4. The recombinant binding protein according to E2 or E3, wherein said first ankyrin repeat module comprises an amino acid sequence selected from the group consisting of (1) SEQ ID NO: 30 and (2) sequences in which up to 9 amino acids in SEQ ID NO: 30 are substituted by other amino acids, and wherein said second ankyrin repeat module comprises an amino acid sequence selected from the group consisting of (1) SEQ ID NO: 31 and (2) sequences in which up to 9 amino acids of SEQ ID NO: 31 are substituted by other amino acids.
E5. The recombinant binding protein according to E2, wherein said ankyrin repeat domain further comprises a third ankyrin repeat module having an amino acid sequence selected from the group consisting of (1) SEQ ID NOs: 27 to 41 and (2) sequences in which up to 9 amino acids in any of SEQ ID NOs: 27 to 41 are substituted by other amino acids.
E6. The recombinant binding protein according to E5, wherein said first, second and third ankyrin repeat modules each independently comprise an amino acid sequence selected from the group consisting of (1) SEQ ID NO: 27 to 29 and 32 to 34 and (2) sequences in which up to 9 amino acids in any of SEQ ID NO: 27 to 29 and 32 to 34 are substituted by other amino acids.
E7. The recombinant binding protein according to any one of E2 to E4, wherein said first ankyrin repeat module is located N-terminally of said second ankyrin repeat module within said ankyrin repeat domain.
E8. The recombinant binding protein according to any one of E5 to E6, wherein said first ankyrin repeat module is located N-terminally of said second ankyrin repeat module, and said second ankyrin repeat module is located N-terminally of said third ankyrin repeat module within said ankyrin repeat domain.
E9. A recombinant binding protein comprising an ankyrin repeat domain with binding specificity for CD16a, wherein said ankyrin repeat domain comprises an amino acid sequence with at least 80% amino acid sequence identity with any one of SEQ ID NOs: 1 to 7, and wherein A at the second last position of SEQ ID NOs: 1 to 7 is optionally substituted by L, and/or A at the last position of SEQ ID NOs: 1 to 7 is optionally substituted by N.
E10. The recombinant binding protein according to E9, wherein said ankyrin repeat domain comprises an amino acid sequence with at least 80% amino acid sequence identity with SEQ ID NO: 2, and wherein A at the second last position of SEQ ID NO: 2 is optionally substituted by L, and/or A at the last position of SEQ ID NO: 2 is optionally substituted by N.
E11. The recombinant binding protein according to any one of the preceding embodiments, wherein said recombinant binding protein binds human CD16a in PBS with a dissociation constant (KD) below 10⁻⁷M.
E12. The recombinant binding protein according to any one of the preceding embodiments, wherein said recombinant binding protein does not substantially bind human CD16b or does not bind to human CD16b in PBS with a dissociation constant (KD) below 10⁻⁷M.
E13. The recombinant binding protein according to any one of the preceding embodiments, wherein said recombinant binding protein further comprises one, two or three binding moieties, wherein each of said binding moieties has binding specificity for a disease-associated antigen.
E14. The recombinant binding protein according to E13, wherein at least one of said binding moieties with binding specificity for a disease-associated antigen is an ankyrin repeat domain.
E15. The recombinant binding protein according to any one of the preceding embodiments, wherein said recombinant binding protein further comprises a half-life extending moiety.
E16. A nucleic acid encoding the recombinant binding protein according to any one of the preceding embodiments.
E17. A pharmaceutical composition comprising the recombinant binding protein according to any one of E1 to E15 or the nucleic acid according to E16, and optionally a pharmaceutically acceptable carrier and/or diluent.
E18. A method of activating immune cells, the method comprising the step of administering to a subject in need thereof an effective amount of the recombinant binding protein according to any one of E1 to E15, the nucleic acid according to E16 or the pharmaceutical composition according to E17.
E19. The method according to E18, wherein said immune cells are innate immune cells.
E20. The method according to E19, wherein said immune cells are natural killer cells.
E21. A method of treating a medical condition, the method comprising the step of administering to a subject in need thereof a therapeutically effective amount of the recombinant binding protein according to any one of E1 to E15, the nucleic acid of E16 or the pharmaceutical composition of E17.
E22. The method according to E21, wherein said medical condition is a cancer.
E23. The recombinant binding protein according to any one of E1 to E15, the nucleic acid according to E16 or the pharmaceutical composition according to E17 for use in the treatment of a medical condition.
E24. The recombinant binding protein according to E23, the nucleic acid according to E23 or the pharmaceutical composition according to E23, wherein said medical condition is a cancer.
E25. Use of the recombinant binding protein according to any one of E1 to E15, the nucleic acid according to E16 or the pharmaceutical composition according to E17 in the treatment of a medical condition.
E26. The use according to E25, wherein said medical condition is a cancer.
E27. Use of the recombinant binding protein according to any one of E1 to E15, the nucleic acid according to E16 or the pharmaceutical composition according to E17 in the manufacture of a medicament for the treatment of a medical condition.
E28. The use according to E27, wherein said medical condition is a cancer.
E29. A method for producing a recombinant binding protein according to any one of E1 to E15, said method comprising the steps of (i) expressing said recombinant binding protein in a suitable host cell, and (ii) purifying said recombinant binding protein.

### BRIEF DESCRIPTION OF THE FIGURES

FIG. 1: Binding specificity profile of CD16a-specific DARPins formatted as mono-specific (CD16a) or bi-specific constructs (CD16a combined to a HER2-specific ankyrin repeat domain of SEQ ID NO: 9). FIG. 1A shows the expression of CD16a on the surface of Jurkat-Lucia-NFAT cells and CD16b on the surface of transfected WT Jurkat cells stably expressing CD16b, as measured by flow cytometry. Serial dilutions of the constructs comprising CD16a-specific DARPins were incubated with these target cells, and binding of the constructs to the target cells was determined by flow cytometry. FIG. 1B shows the measured median fluorescence intensity (MFI) values for Construct 1 comprising DARPin #2 (SEQ ID NO: 2), Construct 2 comprising DARPin #3 (SEQ ID NO: 3), Construct 3 of SEQ ID NO: 13 which comprises DARPin #2 and a C-terminal HER2-specific domain, and Construct 4 of SEQ ID NO: 15 which comprises DARPin #3 and a N-terminal HER2-specific domain. FIG. 1C shows the measured MFI values for Construct 5 of SEQ ID NO: 11 which comprises DARPin #2 and a N-terminal HER2-specific domain, Construct 6 of SEQ ID NO: 17 which comprises DARPin #3 and a C-terminal HER2-specific domain, Construct 16 of SEQ ID NO: 19 which comprises DARPin #4 (SEQ ID NO: 4) and a N-terminal HER2-specific domain, Construct 17 of SEQ ID NO: 20 which comprises DARPin #5 (SEQ ID NO: 5) and a N-terminal HER2-specific domain and Construct 18 of SEQ ID NO: 21 which comprises DARPin #6 (SEQ ID NO: 6) and a N-terminal HER2-specific domain. FIG. 1D shows the measured MFI values for Construct of SEQ ID NO: 10 which comprises DARPin #2, Construct of SEQ ID NO: 11 which comprises DARPin #2, Construct of SEQ ID NO: 12 which comprises DARPin #2, and Construct of SEQ ID NO: 13 which comprises DARPin #2.

FIG. 2: Functional test results of CD16a-specific DARPins formatted as bi-specific (or 2D) NK cell engager constructs (CD16a combined to a HER2-specific ankyrin repeat domain) in an ADCC reporter cell assay. FIG. 2A shows a schematic view of the assay, where Jurkat-NFAT reporter cells emit luminescence upon binding of CD16aV receptor expressed on their surface (CD16aV refers to a CD16a sequence having a Val at position 176 (when counting the signal sequence). This corresponds to Val in position 160 of SEQ ID NO: 72). The 2D constructs are co-incubated with the reporter cells and tumor cells which express HER2, and subsequent signal is quantified. In FIG. 2B, SKOV3 target cells were used (E:T ratio of 5:1) to test Constructs comprising DARPin #2 (i.e Constructs No. 3, 5, 7 and 8), Constructs comprising DARPin #3 (i.e Constructs No. 4, 6, 9 and 10) and Construct comprising DARPin #1 (i.e Construct No. 11). Sequence details of those construct are shown in Table 3. Corresponding mono-specific (or 1D) HER2 binders were used as negative controls (i.e Constructs 12 and 13) and Trastuzumab as a reference HER2 binder. A control in which the Constructs were incubated with the reporter cells but without tumor cells is also shown. FIGS. 2C and 2D show results of further similar ADCC assays in which various HER2 expressing tumor cells were used as target cells. FIG. 2C shows the HER2 expression profile of said cell lines (A: SKOV3, B: JIMT-1, C: MDA-MB-175, D: HT29, E: MDA-MB-231 and F: MDA-MB-468) measured by flow cytometry. FIG. 2D shows the measured signals for Constructs No. 3 (SEQ ID NO: 13), 6 (SEQ ID NO: 17) and 11 (SEQ ID NO: 18) for each assay using the indicated cell lines. Corresponding mono-specific (or 1D) HER2 binders (Construct No. 13) were used as negative controls and Trastuzumab as a reference HER2 binder. Further sequence details are shown in Table 4a. FIG. 2E shows the effect of a construct with SEQ ID NO: 10 upon incubation with the reporter cells expressing CD32a and tumor cells which express HER2. FIG. 2F FIG. 2E shows the effect of a construct with SEQ ID NO: 10 upon incubation with the reporter cells expressing CD16a and tumor cells which express HER2.

FIG. 3: Activation of isolated human primary NK cells by CD16a DARPins formatted as bi-specific (2D) NK cell engagers constructs (CD16a combined to a HER2-specific ankyrin repeat domain). Constructs No. 3 (SEQ ID NO: 13) and 4 (SEQ ID NO: 17) next to mono-specific (or 1D) HER2 binders (Construct No. 13) and Trastuzumab were tested on HER2-expressing SKOV3 cells with a E:T ratio of 5:1 (E=primary NK cells and T=SKOV3 cells). Sequence details of the constructs are shown in Table 5. Various NK cell activation readouts were measured: IFNγ and granzyme B secretion (ELISA) is shown in FIG. 3A, percent of CD25 and CD69 expression (flow cytometry) is shown in FIG. 3B.

FIG. 4: Potency test results of CD16a-specific DARPins formatted as bi-specific (or 2D) NK cell engager constructs (CD16a combined to a HER2-specific ankyrin repeat domain) in tumor cell killing assays with primary human NK-cell and HER2 expressing tumor cells are shown. Readout of the potency was done by LDH release quantification. In FIG. 4A, SKOV3 target cells were incubated (E:T ratio of 5:1) with Constructs comprising DARPin #2 (i.e. Constructs No. 3, 5, 7 and 8), Constructs comprising DARPin #3 (i.e Constructs No. 4, 6, 9 and 10) or Construct comprising DARPin #1 (i.e. Construct No. 11). Sequence details of those construct are shown in Table 3. Corresponding mono-specific (or 1D) HER2 binders were used as negative controls (i.e. Constructs 12 and 13) and trastuzumab as a reference HER2 binder. A control in which the Constructs were incubated with the primary NK cells but without tumor cells is also shown. FIG. 4B show results of further similar tumor cell killing assays in which various HER2 expressing tumor cells were used as target cells. HER2 expression profile of said cell lines (SKOV3, JIMT1, MDA-MB-175, HT29, MDA-MB-231 and MDA-MB-468) correspond to the FIG. 2C. Measured LDH release for Constructs No. 3 (SEQ ID NO: 13), 6 (SEQ ID NO: 17) and 11 (SEQ ID NO: 18) for each assay using the indicated cell lines are shown. Corresponding mono-specific (or 1D) HER2 binders (Construct No. 13) were used as negative controls and trastuzumab as a reference HER2 binder. Further sequence details are shown in Table 4a.

FIG. 5: Surface Plasmon Resonance (SPR) analysis of binding proteins binding to human CD16a, exemplified by DARPin #2. Various concentrations (100 nM starting conc. and 1:3 dilutions) of purified ankyrin repeat protein were applied to a NLG chip with immobilized human CD16a (allotype 165V) for on-rate and off-rate measurements. The obtained SPR trace analyses were used to determine the binding affinity of the ankyrin repeat protein to CD16a. RU, Resonance Units.

FIG. 6: Serum concentration-time traces of CD16a specific Construct 14 (SEQ ID NO: 22) and Construct 15 (SEQ ID NO: 23) comprising respectively DARPin #2 and #3 in mice following intravenous administration of 1 mg/kg of the constructs (mean+/−SD, n=3 per time point, n=6 at last time point). Both Constructs also comprise a human serum albumin (HSA) binding ankyrin repeat domain of SEQ ID NO: 24.

FIG. 7: Cross-reactivity of DARPin #2 and DARPin #7 for human or cynomolgus CD16a target was determined by SPR, using titrations of the purified DARPins (100 nM starting conc. and 1:3 dilutions). Measured K_{D} values are shown in Table 8. RU, Resonance Units.

FIG. 8: Assessment of DARPin-dependent cellular cytotoxicity (DDCC). Bispecific (or 2D) immune cell engager constructs comprising CD16a-binding DARPin and CD117-binding ankyrin repeat domain were tested in a DDCC reporter assay on Kasumi-1 (CD117⁺) target cells (upper plot). A control in which the constructs were incubated with the reporter cells but without target cells is also shown (lower plot). Sequence details of the constructs and measured EC50 values are provided in Table 10.

FIG. 9: NK cell degranulation assay. Bispecific (or 2D) immune cell engager constructs comprising CD16a-binding DARPin and CD117-binding ankyrin repeat domain at different concentrations (10 nM (black), 1 nM (hatched), 0.1 nM (striped) and 0 nM (white)) were tested for their capacity to mediate degranulation (% of CD107a) of isolated primary NK cells (gated as live CD56+) in the presence of Kasumi-1 cells.

FIG. 10: DARPin-dependent cellular phagocytosis assay (DDCP). Bispecific (or 2D) immune cell engager constructs comprising CD16a-binding DARPin and CD117-binding ankyrin repeat domain were tested for their capacity to induce MO macrophages (C11b⁺) to phagocytose cell-tracker labelled Kasumi-1 (CD117⁺) target cells. The tested samples were applied at four different concentrations (10 nM (black), 1 nM (hatched), 0.1 nM (striped) and 0.01 nM (white) in FIG. 10A, or 1 nM (black), 0.2 nM (hatched), 0.04 nM (striped) and 0.008 nM (white) in FIG. 10B to Kasumi-1 target cells. Shown are % of double positive CD11 b+Cell Trace+ cells.

### DETAILED DESCRIPTION OF THE INVENTION

As presented above, CD16a is an attractive therapeutic target for the treatment of certain cancers, particularly leukaemia such as AML. The recombinant binding proteins described herein comprise a designed ankyrin repeat domain and designed ankyrin repeat modules that specifically bind to human CD16a. Furthermore, said recombinant binding proteins do not substantially bind with specificity to CD16b. Also disclosed are nucleic acids encoding the recombinant binding proteins, pharmaceutical compositions comprising the recombinant binding proteins or nucleic acids, and methods of using the recombinant binding proteins, nucleic acids, or pharmaceutical compositions.

Binding proteins specific for CD16a may be particularly useful as a component of a bi- or multi-specific binding molecule that is directed against disease-associated cells, as it would mainly recruit immune cells such as NK cells or macrophages, and would not be bound by circulating soluble CD16b or diverted from NK cell binding by binding to neutrophils or activated eosinophils, which predominantly express CD16b, along with CD32. CD16a and CD16b share a very high sequence identity and differ by only four amino acid residues in the extracellular antibody-binding domains. Despite the significant homology shared by CD16a and CD16b, CD16a binds antibodies more than 10-fold tighter, although it is unclear how differences in these four amino acids contribute to differences in binding affinity (Subedi et al; (2016); mAbs; 8: 1512-1524). Only one of these four differences, at position 129, forms part of the IgG1-binding interface. To efficiently mediate NK cell killing, the CD16a-specific binding protein must not only bind NK cells but also activate them upon binding. Applicant identified, designed, and generated CD16a-specific binding domains, which are able to activate NK cells as described in Example 4, and thus are suitable to be used as building blocks in therapeutic agents, such as, e.g., multi-specific binding proteins having beneficial properties. As the result of a complex screening process and rational design, multiple CD16a-specific ankyrin repeat proteins were generated and produced, namely DARPin proteins comprising any one of SEQ ID NO: 1 to 7.

### Ankyrin Repeat Domains

Designed ankyrin repeat protein libraries (WO2002/020565; Binz et al., Nat. Biotechnol. 22, 575-582, 2004; Stumpp et al., Drug Discov. Today 13, 695-701, 2008) can be used for the selection of target-specific designed ankyrin repeat domains that bind to their target with high affinity. Such target-specific designed ankyrin repeat domains in turn can be used as valuable components of recombinant binding proteins for the treatment of diseases. Designed ankyrin repeat proteins are a class of binding molecules which have the potential to overcome limitations of monoclonal antibodies, hence allowing novel therapeutic approaches. Such ankyrin repeat proteins may comprise a single designed ankyrin repeat domain or may comprise a combination of two or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two or more designed ankyrin repeat domains in one protein, make designed ankyrin repeat proteins ideal agonistic, antagonistic and/or inhibitory drug candidates. Furthermore, such ankyrin repeat proteins can be engineered to carry various effector functions, e.g., cytotoxic agents and/or half-life extending agents, enabling completely new drug formats. Such drug formats include e.g. innate immune cell engager molecules in which a CD16a-specific designed ankyrin repeat domain is combined to one, two, three or more binding moieties with defined target specificities forming a multi-specific binding protein engaging the CD16a receptor on innate immune cells such as NK cells or macrophages in combination with the other targets, as further described herein below. By simultaneously targeting antigens on target cells, e.g. tumor cells, and activating receptors on innate immune cells, such innate immune cell engager molecules strengthen the interaction between the two cell types and increase innate immune cell effector functions. Target cell-localized, e.g. tumor-localized, activation of the innate immune cells can therefore be achieved.

In one aspect, the invention relates to a recombinant binding protein comprising an ankyrin repeat domain, wherein said ankyrin repeat domain has binding specificity for CD16a, and wherein said ankyrin repeat domain comprises an ankyrin repeat module having an amino acid sequence selected from the group consisting of (1) SEQ ID NOs: 27 to 41 and (2) sequences in which up to 9, up to 8, up to 7, up to 6, up to 5, up to 4, up to 3, up to 2 or up to 1 amino acid(s) in any of SEQ ID NOs: 27 to 41 are substituted by other amino acids. Thus, in one embodiment, said ankyrin repeat module comprises an amino acid sequence selected from the group consisting of (1) SEQ ID NOs: 27 to 41 and (2) sequences in which up to 9 amino acids in any of SEQ ID NOs: 27 to 41 are substituted by other amino acids. In one embodiment, said ankyrin repeat module comprises an amino acid sequence selected from the group consisting of (1) SEQ ID NOs: 27 to 41 and (2) sequences in which up to 3 amino acids in any of SEQ ID NOs: 27 to 41 are substituted by other amino acids. In one embodiment, said ankyrin repeat module comprises an amino acid sequence selected from the group consisting of (1) SEQ ID NOs: 27 to 41 and (2) sequences in which up to 2 amino acids in any of SEQ ID NOs: 27 to 41 are substituted by other amino acids. In one embodiment, said ankyrin repeat module comprises an amino acid sequence selected from the group consisting of (1) SEQ ID NOs: 27 to 41 and (2) sequences in which up to 1 amino acid in any of SEQ ID NOs: 27 to 41 are substituted by other amino acids. In one embodiment, all of said 9, 8, 7, 6, 5, 4, 3, 2, or 1 amino acid substitutions occur in framework positions of said ankyrin repeat module(s). In one embodiment, said ankyrin repeat module has an amino acid sequence selected from the group consisting of SEQ ID NOs: 27 to 41.

In one embodiment, said ankyrin repeat module comprises the amino acid sequence of SEQ ID NO: 27 or a sequence in which one or two amino acids in SEQ ID NO: 27 are substituted by other amino acids.

In one embodiment, said ankyrin repeat module comprises the amino acid sequence of SEQ ID NO: 28 or a sequence in which one or two amino acids in SEQ ID NO: 28 are substituted by other amino acids.

In one embodiment, said ankyrin repeat module comprises the amino acid sequence of SEQ ID NO: 29 or a sequence in which one or two amino acids in SEQ ID NO: 29 are substituted by other amino acids.

In one embodiment, said ankyrin repeat module comprises the amino acid sequence of SEQ ID NO: 30 or a sequence in which one or two amino acids in SEQ ID NO: 30 are substituted by other amino acids.

In one embodiment, said ankyrin repeat module comprises the amino acid sequence of SEQ ID NO: 31 or a sequence in which one or two amino acids in SEQ ID NO: 31 are substituted by other amino acids.

In one embodiment, said ankyrin repeat module comprises the amino acid sequence of SEQ ID NO: 32 or a sequence in which one or two amino acids in SEQ ID NO: 32 are substituted by other amino acids.

In one embodiment, said ankyrin repeat module comprises the amino acid sequence of SEQ ID NO: 33 or a sequence in which one or two amino acids in SEQ ID NO: 33 are substituted by other amino acids.

In one embodiment, said ankyrin repeat module comprises the amino acid sequence of SEQ ID NO: 34 or a sequence in which one or two amino acids in SEQ ID NO: 34 are substituted by other amino acids.

In one embodiment, said ankyrin repeat module comprises the amino acid sequence of SEQ ID NO: 35 or a sequence in which one or two amino acids in SEQ ID NO: 35 are substituted by other amino acids.

In one embodiment, said ankyrin repeat module comprises the amino acid sequence of SEQ ID NO: 36 or a sequence in which one or two amino acids in SEQ ID NO: 36 are substituted by other amino acids.

In one embodiment, said ankyrin repeat module comprises the amino acid sequence of SEQ ID NO: 37 or a sequence in which one or two amino acids in SEQ ID NO: 37 are substituted by other amino acids.

In one embodiment, said ankyrin repeat module comprises the amino acid sequence of SEQ ID NO: 38 or a sequence in which one or two amino acids in SEQ ID NO: 38 are substituted by other amino acids.

In one embodiment, said ankyrin repeat module comprises the amino acid sequence of SEQ ID NO: 39 or a sequence in which one or two amino acids in SEQ ID NO: 39 are substituted by other amino acids.

In one embodiment, said ankyrin repeat module comprises the amino acid sequence of SEQ ID NO: 40 or a sequence in which one or two amino acids in SEQ ID NO: 40 are substituted by other amino acids.

In one embodiment, said ankyrin repeat module comprises the amino acid sequence of SEQ ID NO: 41 or a sequence in which one or two amino acids in SEQ ID NO: 41 are substituted by other amino acids.

In one embodiment, said ankyrin repeat module comprises the amino acid sequence of SEQ ID NO: 27. In one embodiment, said ankyrin repeat module comprises the amino acid sequence of SEQ ID NO: 28. In one embodiment, said ankyrin repeat module comprises the amino acid sequence of SEQ ID NO: 29. In one embodiment, said ankyrin repeat module comprises the amino acid sequence of SEQ ID NO: 30. In one embodiment, said ankyrin repeat module comprises the amino acid sequence of SEQ ID NO: 31. In one embodiment, said ankyrin repeat module comprises the amino acid sequence of SEQ ID NO: 32. In one embodiment, said ankyrin repeat module comprises the amino acid sequence of SEQ ID NO: 33. In one embodiment, said ankyrin repeat module comprises the amino acid sequence of SEQ ID NO: 34. In one embodiment, said ankyrin repeat module comprises the amino acid sequence of SEQ ID NO: 35. In one embodiment, said ankyrin repeat module comprises the amino acid sequence of SEQ ID NO: 36. In one embodiment, said ankyrin repeat module comprises the amino acid sequence of SEQ ID NO: 37. In one embodiment, said ankyrin repeat module comprises the amino acid sequence of SEQ ID NO: 38. In one embodiment, said ankyrin repeat module comprises the amino acid sequence of SEQ ID NO: 39. In one embodiment, said ankyrin repeat module comprises the amino acid sequence of SEQ ID NO: 40. In one embodiment, said ankyrin repeat module comprises the amino acid sequence of SEQ ID NO: 41.

In one embodiment, said ankyrin repeat domain comprises a first ankyrin repeat module and a second ankyrin repeat module. In a preferred embodiment, said first ankyrin repeat module is located N-terminally of said second ankyrin repeat module within said ankyrin repeat domain. In one embodiment, said first and said second ankyrin repeat module each comprises an amino acid sequence selected from the group consisting of (1) SEQ ID NOs: 27 to 41 and (2) sequences in which up to 9, up to 8, up to 7, up to 6, up to 5, up to 4, up to 3, up to 2 or up to 1 amino acids in any of SEQ ID NOs: 27 to 41 are substituted by other amino acids. Thus, in one embodiment, said ankyrin repeat domain comprises a first ankyrin repeat module having an amino acid sequence selected from the group consisting of (1) SEQ ID NOs: 27 to 41 and (2) sequences in which up to 9 amino acids in any of SEQ ID NOs: 27 to 41 are substituted by other amino acids and further comprises a second ankyrin repeat module having an amino acid sequence selected from the group consisting of (1) SEQ ID NOs: 27 to 41 and (2) sequences in which up to 9 amino acids in any of SEQ ID NOs: 27 to 41 are substituted by other amino acids.

In one particular embodiment, in such an ankyrin repeat domain said first ankyrin repeat module comprises an amino acid sequence selected from the group consisting of (1) SEQ ID NO: 30 and (2) sequences in which up to 9, up to 8, up to 7, up to 6, up to 5, up to 4, up to 3, up to 2 or up to 1 amino acids in SEQ ID NO: 30 are substituted by other amino acids, and said second ankyrin repeat module comprises an amino acid sequence selected from the group consisting of (1) SEQ ID NO: 31 and (2) sequences in which up to 9, up to 8, up to 7, up to 6, up to 5, up to 4, up to 3, up to 2 or up to 1 amino acids of SEQ ID NO: 31 are substituted by other amino acids.

Thus, in one embodiment, in such an ankyrin repeat domain said first ankyrin repeat module comprises an amino acid sequence selected from the group consisting of (1) SEQ ID NO: 30 and (2) sequences in which up to 9 amino acids in SEQ ID NO: 30 are substituted by other amino acids, and said second ankyrin repeat module comprises an amino acid sequence selected from the group consisting of (1) SEQ ID NO: 31 and (2) sequences in which up to 9 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 30 and (2) sequences in which up to 6 amino acids in SEQ ID NO: 30 are substituted by other amino acids, and said second ankyrin repeat module comprises an amino acid sequence selected from the group consisting of (1) SEQ ID NO: 31 and (2) sequences in which up to 6 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 30 and (2) sequences in which up to 5 amino acids in SEQ ID NO: 30 are substituted by other amino acids, and said second ankyrin repeat module comprises an amino acid sequence selected from the group consisting of (1) SEQ ID NO: 31 and (2) sequences in which up to 5 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 30 and (2) sequences in which up to 4 amino acids in SEQ ID NO: 30 are substituted by other amino acids, and said second ankyrin repeat module comprises an amino acid sequence selected from the group consisting of (1) SEQ ID NO: 31 and (2) sequences in which up to 4 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 30 and (2) sequences in which up to 3 amino acids in SEQ ID NO: 30 are substituted by other amino acids, and said second ankyrin repeat module comprises an amino acid sequence selected from the group consisting of (1) SEQ ID NO: 31 and (2) sequences in which up to 3 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 30 and (2) sequences in which up to 2 amino acids in SEQ ID NO: 30 are substituted by other amino acids, and said second ankyrin repeat module comprises an amino acid sequence selected from the group consisting of (1) SEQ ID NO: 31 and (2) sequences in which up to 2 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 30 and (2) sequences in which 1 amino acid in SEQ ID NO: 30 is substituted by another amino acid, and said second ankyrin repeat module comprises an amino acid sequence selected from the group consisting of (1) SEQ ID NO: 31 and (2) sequences in which 1 amino acid of SEQ ID NO: 31 is substituted by another amino acid. In one embodiment, in such an ankyrin repeat domain said first ankyrin repeat module comprises the amino acid sequence of SEQ ID NO: 30 and said second ankyrin repeat module comprises the amino acid sequence of SEQ ID NO: 31.

In a preferred embodiment, said first ankyrin repeat module comprises an amino acid sequence selected from the group consisting of (1) SEQ ID NO: 30 and (2) sequences in which up to 3, up to 2 or up to 1 amino acids in SEQ ID NO: 30 are substituted by other amino acids, and said second ankyrin repeat module comprises an amino acid sequence selected from the group consisting of (1) SEQ ID NO: 31 and (2) sequences in which up to 3, up to 2 or up to 1 amino acids of SEQ ID NO: 31 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 30 and said second ankyrin repeat module comprises the amino acid sequence of SEQ ID NO: 31, wherein said first ankyrin repeat module is located N-terminally of said second ankyrin repeat module within said ankyrin repeat domain.

In one particular embodiment, in such an ankyrin repeat domain said first ankyrin repeat module comprises an amino acid sequence selected from the group consisting of (1) SEQ ID NO: 35 and (2) sequences in which up to 9, up to 8, up to 7, up to 6, up to 5, up to 4, up to 3, up to 2 or up to 1 amino acids in SEQ ID NO: 35 are substituted by other amino acids, and said second ankyrin repeat module comprises an amino acid sequence selected from the group consisting of (1) SEQ ID NO: 36 and (2) sequences in which up to 9, up to 8, up to 7, up to 6, up to 5, up to 4, up to 3, up to 2 or up to 1 amino acids of SEQ ID NO: 36 are substituted by other amino acids.

Thus, in one embodiment, in such an ankyrin repeat domain said first ankyrin repeat module comprises an amino acid sequence selected from the group consisting of (1) SEQ ID NO: 35 and (2) sequences in which up to 9 amino acids in SEQ ID NO: 35 are substituted by other amino acids, and said second ankyrin repeat module comprises an amino acid sequence selected from the group consisting of (1) SEQ ID NO: 36 and (2) sequences in which up to 9 amino acids of SEQ ID NO: 36 are substituted by other amino acids. In one embodiment, in such an ankyrin repeat domain said first ankyrin repeat module comprises an amino acid sequence selected from the group consisting of (1) SEQ ID NO: 35 and (2) sequences in which up to 6 amino acids in SEQ ID NO: 35 are substituted by other amino acids, and said second ankyrin repeat module comprises an amino acid sequence selected from the group consisting of (1) SEQ ID NO: 36 and (2) sequences in which up to 6 amino acids of SEQ ID NO: 36 are substituted by other amino acids. In one embodiment, in such an ankyrin repeat domain said first ankyrin repeat module comprises an amino acid sequence selected from the group consisting of (1) SEQ ID NO: 35 and (2) sequences in which up to 5 amino acids in SEQ ID NO: 35 are substituted by other amino acids, and said second ankyrin repeat module comprises an amino acid sequence selected from the group consisting of (1) SEQ ID NO: 36 and (2) sequences in which up to 5 amino acids of SEQ ID NO: 36 are substituted by other amino acids. In one embodiment, in such an ankyrin repeat domain said first ankyrin repeat module comprises an amino acid sequence selected from the group consisting of (1) SEQ ID NO: 35 and (2) sequences in which up to 4 amino acids in SEQ ID NO: 35 are substituted by other amino acids, and said second ankyrin repeat module comprises an amino acid sequence selected from the group consisting of (1) SEQ ID NO: 36 and (2) sequences in which up to 4 amino acids of SEQ ID NO: 36 are substituted by other amino acids. In one embodiment, in such an ankyrin repeat domain said first ankyrin repeat module comprises an amino acid sequence selected from the group consisting of (1) SEQ ID NO: 35 and (2) sequences in which up to 3 amino acids in SEQ ID NO: 35 are substituted by other amino acids, and said second ankyrin repeat module comprises an amino acid sequence selected from the group consisting of (1) SEQ ID NO: 36 and (2) sequences in which up to 3 amino acids of SEQ ID NO: 36 are substituted by other amino acids. In one embodiment, in such an ankyrin repeat domain said first ankyrin repeat module comprises an amino acid sequence selected from the group consisting of (1) SEQ ID NO: 35 and (2) sequences in which up to 2 amino acids in SEQ ID NO: 35 are substituted by other amino acids, and said second ankyrin repeat module comprises an amino acid sequence selected from the group consisting of (1) SEQ ID NO: 36 and (2) sequences in which up to 2 amino acids of SEQ ID NO: 36 are substituted by other amino acids. In one embodiment, in such an ankyrin repeat domain said first ankyrin repeat module comprises an amino acid sequence selected from the group consisting of (1) SEQ ID NO: 35 and (2) sequences in which 1 amino acid in SEQ ID NO: 35 is substituted by another amino acid, and said second ankyrin repeat module comprises an amino acid sequence selected from the group consisting of (1) SEQ ID NO: 36 and (2) sequences in which 1 amino acid of SEQ ID NO: 36 is substituted by another amino acid. In one embodiment, in such an ankyrin repeat domain said first ankyrin repeat module comprises the amino acid sequence of SEQ ID NO: 35 and said second ankyrin repeat module comprises the amino acid sequence of SEQ ID NO: 36.

In a preferred embodiment, said first ankyrin repeat module comprises an amino acid sequence selected from the group consisting of (1) SEQ ID NO: 35 and (2) sequences in which up to 3, up to 2 or up to 1 amino acids in SEQ ID NO: 35 are substituted by other amino acids, and said second ankyrin repeat module comprises an amino acid sequence selected from the group consisting of (1) SEQ ID NO: 36 and (2) sequences in which up to 3, up to 2 or up to 1 amino acids of SEQ ID NO: 36 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 35 and said second ankyrin repeat module comprises the amino acid sequence of SEQ ID NO: 36, wherein said first ankyrin repeat module is located N-terminally of said second ankyrin repeat module within said ankyrin repeat domain.

In one particular embodiment, in such an ankyrin repeat domain said first ankyrin repeat module comprises an amino acid sequence selected from the group consisting of (1) SEQ ID NO: 37 and (2) sequences in which up to 9, up to 8, up to 7, up to 6, up to 5, up to 4, up to 3, up to 2 or up to 1 amino acids in SEQ ID NO: 37 are substituted by other amino acids, and said second ankyrin repeat module comprises an amino acid sequence selected from the group consisting of (1) SEQ ID NO: 38 and (2) sequences in which up to 9, up to 8, up to 7, up to 6, up to 5, up to 4, up to 3, up to 2 or up to 1 amino acids of SEQ ID NO: 38 are substituted by other amino acids.

Thus, in one embodiment, in such an ankyrin repeat domain said first ankyrin repeat module comprises an amino acid sequence selected from the group consisting of (1) SEQ ID NO: 37 and (2) sequences in which up to 9 amino acids in SEQ ID NO: 37 are substituted by other amino acids, and said second ankyrin repeat module comprises an amino acid sequence selected from the group consisting of (1) SEQ ID NO: 38 and (2) sequences in which up to 9 amino acids of SEQ ID NO: 38 are substituted by other amino acids. In one embodiment, in such an ankyrin repeat domain said first ankyrin repeat module comprises an amino acid sequence selected from the group consisting of (1) SEQ ID NO: 37 and (2) sequences in which up to 6 amino acids in SEQ ID NO: 37 are substituted by other amino acids, and said second ankyrin repeat module comprises an amino acid sequence selected from the group consisting of (1) SEQ ID NO: 38 and (2) sequences in which up to 6 amino acids of SEQ ID NO: 38 are substituted by other amino acids. In one embodiment, in such an ankyrin repeat domain said first ankyrin repeat module comprises an amino acid sequence selected from the group consisting of (1) SEQ ID NO: 37 and (2) sequences in which up to 5 amino acids in SEQ ID NO: 37 are substituted by other amino acids, and said second ankyrin repeat module comprises an amino acid sequence selected from the group consisting of (1) SEQ ID NO: 38 and (2) sequences in which up to 5 amino acids of SEQ ID NO: 38 are substituted by other amino acids. In one embodiment, in such an ankyrin repeat domain said first ankyrin repeat module comprises an amino acid sequence selected from the group consisting of (1) SEQ ID NO: 37 and (2) sequences in which up to 4 amino acids in SEQ ID NO: 37 are substituted by other amino acids, and said second ankyrin repeat module comprises an amino acid sequence selected from the group consisting of (1) SEQ ID NO: 38 and (2) sequences in which up to 4 amino acids of SEQ ID NO: 38 are substituted by other amino acids. In one embodiment, in such an ankyrin repeat domain said first ankyrin repeat module comprises an amino acid sequence selected from the group consisting of (1) SEQ ID NO: 37 and (2) sequences in which up to 3 amino acids in SEQ ID NO: 37 are substituted by other amino acids, and said second ankyrin repeat module comprises an amino acid sequence selected from the group consisting of (1) SEQ ID NO: 38 and (2) sequences in which up to 3 amino acids of SEQ ID NO: 38 are substituted by other amino acids. In one embodiment, in such an ankyrin repeat domain said first ankyrin repeat module comprises an amino acid sequence selected from the group consisting of (1) SEQ ID NO: 37 and (2) sequences in which up to 2 amino acids in SEQ ID NO: 37 are substituted by other amino acids, and said second ankyrin repeat module comprises an amino acid sequence selected from the group consisting of (1) SEQ ID NO: 38 and (2) sequences in which up to 2 amino acids of SEQ ID NO: 38 are substituted by other amino acids. In one embodiment, in such an ankyrin repeat domain said first ankyrin repeat module comprises an amino acid sequence selected from the group consisting of (1) SEQ ID NO: 37 and (2) sequences in which 1 amino acid in SEQ ID NO: 37 is substituted by another amino acid, and said second ankyrin repeat module comprises an amino acid sequence selected from the group consisting of (1) SEQ ID NO: 38 and (2) sequences in which 1 amino acid of SEQ ID NO: 38 is substituted by another amino acid. In one embodiment, in such an ankyrin repeat domain said first ankyrin repeat module comprises the amino acid sequence of SEQ ID NO: 37 and said second ankyrin repeat module comprises the amino acid sequence of SEQ ID NO: 38.

In a preferred embodiment, said first ankyrin repeat module comprises an amino acid sequence selected from the group consisting of (1) SEQ ID NO: 37 and (2) sequences in which up to 3, up to 2 or up to 1 amino acids in SEQ ID NO: 37 are substituted by other amino acids, and said second ankyrin repeat module comprises an amino acid sequence selected from the group consisting of (1) SEQ ID NO: 38 and (2) sequences in which up to 3, up to 2 or up to 1 amino acids of SEQ ID NO: 38 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 37 and said second ankyrin repeat module comprises the amino acid sequence of SEQ ID NO: 38, wherein said first ankyrin repeat module is located N-terminally of said second ankyrin repeat module within said ankyrin repeat domain.

In one particular embodiment, in such an ankyrin repeat domain said first ankyrin repeat module comprises an amino acid sequence selected from the group consisting of (1) SEQ ID NO: 39 and (2) sequences in which up to 9, up to 8, up to 7, up to 6, up to 5, up to 4, up to 3, up to 2 or up to 1 amino acids in SEQ ID NO: 39 are substituted by other amino acids, and said second ankyrin repeat module comprises an amino acid sequence selected from the group consisting of (1) SEQ ID NO: 40 and (2) sequences in which up to 9, up to 8, up to 7, up to 6, up to 5, up to 4, up to 3, up to 2 or up to 1 amino acids of SEQ ID NO: 40 are substituted by other amino acids.

Thus, in one embodiment, in such an ankyrin repeat domain said first ankyrin repeat module comprises an amino acid sequence selected from the group consisting of (1) SEQ ID NO: 39 and (2) sequences in which up to 9 amino acids in SEQ ID NO: 39 are substituted by other amino acids, and said second ankyrin repeat module comprises an amino acid sequence selected from the group consisting of (1) SEQ ID NO: 40 and (2) sequences in which up to 9 amino acids of SEQ ID NO: 40 are substituted by other amino acids. In one embodiment, in such an ankyrin repeat domain said first ankyrin repeat module comprises an amino acid sequence selected from the group consisting of (1) SEQ ID NO: 39 and (2) sequences in which up to 6 amino acids in SEQ ID NO: 39 are substituted by other amino acids, and said second ankyrin repeat module comprises an amino acid sequence selected from the group consisting of (1) SEQ ID NO: 40 and (2) sequences in which up to 6 amino acids of SEQ ID NO: 40 are substituted by other amino acids. In one embodiment, in such an ankyrin repeat domain said first ankyrin repeat module comprises an amino acid sequence selected from the group consisting of (1) SEQ ID NO: 39 and (2) sequences in which up to 5 amino acids in SEQ ID NO: 39 are substituted by other amino acids, and said second ankyrin repeat module comprises an amino acid sequence selected from the group consisting of (1) SEQ ID NO: 40 and (2) sequences in which up to 5 amino acids of SEQ ID NO: 40 are substituted by other amino acids. In one embodiment, in such an ankyrin repeat domain said first ankyrin repeat module comprises an amino acid sequence selected from the group consisting of (1) SEQ ID NO: 39 and (2) sequences in which up to 4 amino acids in SEQ ID NO: 39 are substituted by other amino acids, and said second ankyrin repeat module comprises an amino acid sequence selected from the group consisting of (1) SEQ ID NO: 40 and (2) sequences in which up to 4 amino acids of SEQ ID NO: 40 are substituted by other amino acids. In one embodiment, in such an ankyrin repeat domain said first ankyrin repeat module comprises an amino acid sequence selected from the group consisting of (1) SEQ ID NO: 39 and (2) sequences in which up to 3 amino acids in SEQ ID NO: 39 are substituted by other amino acids, and said second ankyrin repeat module comprises an amino acid sequence selected from the group consisting of (1) SEQ ID NO: 40 and (2) sequences in which up to 3 amino acids of SEQ ID NO: 40 are substituted by other amino acids. In one embodiment, in such an ankyrin repeat domain said first ankyrin repeat module comprises an amino acid sequence selected from the group consisting of (1) SEQ ID NO: 39 and (2) sequences in which up to 2 amino acids in SEQ ID NO: 39 are substituted by other amino acids, and said second ankyrin repeat module comprises an amino acid sequence selected from the group consisting of (1) SEQ ID NO: 40 and (2) sequences in which up to 2 amino acids of SEQ ID NO: 40 are substituted by other amino acids. In one embodiment, in such an ankyrin repeat domain said first ankyrin repeat module comprises an amino acid sequence selected from the group consisting of (1) SEQ ID NO: 39 and (2) sequences in which 1 amino acid in SEQ ID NO: 39 is substituted by another amino acid, and said second ankyrin repeat module comprises an amino acid sequence selected from the group consisting of (1) SEQ ID NO: 40 and (2) sequences in which 1 amino acid of SEQ ID NO: 40 is substituted by another amino acid. In one embodiment, in such an ankyrin repeat domain said first ankyrin repeat module comprises the amino acid sequence of SEQ ID NO: 39 and said second ankyrin repeat module comprises the amino acid sequence of SEQ ID NO: 40.

In a preferred embodiment, said first ankyrin repeat module comprises an amino acid sequence selected from the group consisting of (1) SEQ ID NO: 39 and (2) sequences in which up to 3, up to 2 or up to 1 amino acids in SEQ ID NO: 39 are substituted by other amino acids, and said second ankyrin repeat module comprises an amino acid sequence selected from the group consisting of (1) SEQ ID NO: 40 and (2) sequences in which up to 3, up to 2 or up to 1 amino acids of SEQ ID NO: 40 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 39 and said second ankyrin repeat module comprises the amino acid sequence of SEQ ID NO: 40, wherein said first ankyrin repeat module is located N-terminally of said second ankyrin repeat module within said ankyrin repeat domain.

In one particular embodiment, in such an ankyrin repeat domain said first ankyrin repeat module comprises an amino acid sequence selected from the group consisting of (1) SEQ ID NO: 41 and (2) sequences in which up to 9, up to 8, up to 7, up to 6, up to 5, up to 4, up to 3, up to 2 or up to 1 amino acids in SEQ ID NO: 41 are substituted by other amino acids, and said second ankyrin repeat module comprises an amino acid sequence selected from the group consisting of (1) SEQ ID NO: 31 and (2) sequences in which up to 9, up to 8, up to 7, up to 6, up to 5, up to 4, up to 3, up to 2 or up to 1 amino acids of SEQ ID NO: 31 are substituted by other amino acids.

Thus, in one embodiment, in such an ankyrin repeat domain said first ankyrin repeat module comprises an amino acid sequence selected from the group consisting of (1) SEQ ID NO: 41 and (2) sequences in which up to 9 amino acids in SEQ ID NO: 41 are substituted by other amino acids, and said second ankyrin repeat module comprises an amino acid sequence selected from the group consisting of (1) SEQ ID NO: 31 and (2) sequences in which up to 9 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 41 and (2) sequences in which up to 6 amino acids in SEQ ID NO: 41 are substituted by other amino acids, and said second ankyrin repeat module comprises an amino acid sequence selected from the group consisting of (1) SEQ ID NO: 31 and (2) sequences in which up to 6 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 41 and (2) sequences in which up to 5 amino acids in SEQ ID NO: 41 are substituted by other amino acids, and said second ankyrin repeat module comprises an amino acid sequence selected from the group consisting of (1) SEQ ID NO: 31 and (2) sequences in which up to 5 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 41 and (2) sequences in which up to 4 amino acids in SEQ ID NO: 41 are substituted by other amino acids, and said second ankyrin repeat module comprises an amino acid sequence selected from the group consisting of (1) SEQ ID NO: 31 and (2) sequences in which up to 4 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 41 and (2) sequences in which up to 3 amino acids in SEQ ID NO: 41 are substituted by other amino acids, and said second ankyrin repeat module comprises an amino acid sequence selected from the group consisting of (1) SEQ ID NO: 31 and (2) sequences in which up to 3 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 41 and (2) sequences in which up to 2 amino acids in SEQ ID NO: 41 are substituted by other amino acids, and said second ankyrin repeat module comprises an amino acid sequence selected from the group consisting of (1) SEQ ID NO: 31 and (2) sequences in which up to 2 amino acids of SEQ ID NO: 31 are substituted by other amino acids. In one embodiment, in such an ankyrin repeat domain said first ankyrin repeat module comprises an amino acid sequence selected from the group consisting of (1) SEQ ID NO: 41 and (2) sequences in which 1 amino acid in SEQ ID NO: 41 is substituted by another amino acid, and said second ankyrin repeat module comprises an amino acid sequence selected from the group consisting of (1) SEQ ID NO: 31 and (2) sequences in which 1 amino acid of SEQ ID NO: 31 is substituted by another amino acid. In one embodiment, in such an ankyrin repeat domain said first ankyrin repeat module comprises the amino acid sequence of SEQ ID NO: 41 and said second ankyrin repeat module comprises the amino acid sequence of SEQ ID NO: 31.

In a preferred embodiment, said first ankyrin repeat module comprises an amino acid sequence selected from the group consisting of (1) SEQ ID NO: 41 and (2) sequences in which up to 3, up to 2 or up to 1 amino acids in SEQ ID NO: 41 are substituted by other amino acids, and said second ankyrin repeat module comprises an amino acid sequence selected from the group consisting of (1) SEQ ID NO: 31 and (2) sequences in which up to 3, up to 2 or up to 1 amino acids of SEQ ID NO: 31 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 41 and said second ankyrin repeat module comprises the amino acid sequence of SEQ ID NO: 31, wherein said first ankyrin repeat module is located N-terminally of said second ankyrin repeat module within said ankyrin repeat domain.

In one particular embodiment, in such an ankyrin repeat domain said first ankyrin repeat module comprises an amino acid sequence selected from the group consisting of (1) SEQ ID NO: 27 and (2) sequences in which up to 9, up to 8, up to 7, up to 6, up to 5, up to 4, up to 3, up to 2 or up to 1 amino acids in SEQ ID NO: 27 are substituted by other amino acids, and said second ankyrin repeat module comprises an amino acid sequence selected from the group consisting of (1) SEQ ID NO: 28 and (2) sequences in which up to 9, up to 8, up to 7, up to 6, up to 5, up to 4, up to 3, up to 2 or up to 1 amino acids of SEQ ID NO: 28 are substituted by other amino acids.

Thus, in one embodiment, in such an ankyrin repeat domain said first ankyrin repeat module comprises an amino acid sequence selected from the group consisting of (1) SEQ ID NO: 27 and (2) sequences in which up to 9 amino acids in SEQ ID NO: 27 are substituted by other amino acids, and said second ankyrin repeat module comprises an amino acid sequence selected from the group consisting of (1) SEQ ID NO: 28 and (2) sequences in which up to 9 amino acids of SEQ ID NO: 28 are substituted by other amino acids. In one embodiment, in such an ankyrin repeat domain said first ankyrin repeat module comprises an amino acid sequence selected from the group consisting of (1) SEQ ID NO: 27 and (2) sequences in which up to 6 amino acids in SEQ ID NO: 27 are substituted by other amino acids, and said second ankyrin repeat module comprises an amino acid sequence selected from the group consisting of (1) SEQ ID NO: 28 and (2) sequences in which up to 6 amino acids of SEQ ID NO: 28 are substituted by other amino acids. In one embodiment, in such an ankyrin repeat domain said first ankyrin repeat module comprises an amino acid sequence selected from the group consisting of (1) SEQ ID NO: 27 and (2) sequences in which up to 5 amino acids in SEQ ID NO: 27 are substituted by other amino acids, and said second ankyrin repeat module comprises an amino acid sequence selected from the group consisting of (1) SEQ ID NO: 28 and (2) sequences in which up to 5 amino acids of SEQ ID NO: 28 are substituted by other amino acids. In one embodiment, in such an ankyrin repeat domain said first ankyrin repeat module comprises an amino acid sequence selected from the group consisting of (1) SEQ ID NO: 27 and (2) sequences in which up to 4 amino acids in SEQ ID NO: 27 are substituted by other amino acids, and said second ankyrin repeat module comprises an amino acid sequence selected from the group consisting of (1) SEQ ID NO: 28 and (2) sequences in which up to 4 amino acids of SEQ ID NO: 28 are substituted by other amino acids. In one embodiment, in such an ankyrin repeat domain said first ankyrin repeat module comprises an amino acid sequence selected from the group consisting of (1) SEQ ID NO: 27 and (2) sequences in which up to 3 amino acids in SEQ ID NO: 27 are substituted by other amino acids, and said second ankyrin repeat module comprises an amino acid sequence selected from the group consisting of (1) SEQ ID NO: 28 and (2) sequences in which up to 3 amino acids of SEQ ID NO: 28 are substituted by other amino acids. In one embodiment, in such an ankyrin repeat domain said first ankyrin repeat module comprises an amino acid sequence selected from the group consisting of (1) SEQ ID NO: 27 and (2) sequences in which up to 2 amino acids in SEQ ID NO: 27 are substituted by other amino acids, and said second ankyrin repeat module comprises an amino acid sequence selected from the group consisting of (1) SEQ ID NO: 28 and (2) sequences in which up to 2 amino acids of SEQ ID NO: 28 are substituted by other amino acids. In one embodiment, in such an ankyrin repeat domain said first ankyrin repeat module comprises an amino acid sequence selected from the group consisting of (1) SEQ ID NO: 27 and (2) sequences in which 1 amino acid in SEQ ID NO: 27 is substituted by another amino acid, and said second ankyrin repeat module comprises an amino acid sequence selected from the group consisting of (1) SEQ ID NO: 28 and (2) sequences in which 1 amino acid of SEQ ID NO: 28 is substituted by another amino acid. In one embodiment, in such an ankyrin repeat domain said first ankyrin repeat module comprises the amino acid sequence of SEQ ID NO: 27 and said second ankyrin repeat module comprises the amino acid sequence of SEQ ID NO: 28.

In a preferred embodiment, said first ankyrin repeat module comprises an amino acid sequence selected from the group consisting of (1) SEQ ID NO: 27 and (2) sequences in which up to 3, up to 2 or up to 1 amino acids in SEQ ID NO: 27 are substituted by other amino acids, and said second ankyrin repeat module comprises an amino acid sequence selected from the group consisting of (1) SEQ ID NO: 28 and (2) sequences in which up to 3, up to 2 or up to 1 amino acids of SEQ ID NO: 28 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 27 and said second ankyrin repeat module comprises the amino acid sequence of SEQ ID NO: 28, wherein said first ankyrin repeat module is located N-terminally of said second ankyrin repeat module within said ankyrin repeat domain.

In one particular embodiment, in such an ankyrin repeat domain said first ankyrin repeat module comprises an amino acid sequence selected from the group consisting of (1) SEQ ID NO: 32 and (2) sequences in which up to 9, up to 8, up to 7, up to 6, up to 5, up to 4, up to 3, up to 2 or up to 1 amino acids in SEQ ID NO: 32 are substituted by other amino acids, and said second ankyrin repeat module comprises an amino acid sequence selected from the group consisting of (1) SEQ ID NO: 33 and (2) sequences in which up to 9, up to 8, up to 7, up to 6, up to 5, up to 4, up to 3, up to 2 or up to 1 amino acids of SEQ ID NO: 33 are substituted by other amino acids.

Thus, in one embodiment, in such an ankyrin repeat domain said first ankyrin repeat module comprises an amino acid sequence selected from the group consisting of (1) SEQ ID NO: 32 and (2) sequences in which up to 9 amino acids in SEQ ID NO: 32 are substituted by other amino acids, and said second ankyrin repeat module comprises an amino acid sequence selected from the group consisting of (1) SEQ ID NO: 33 and (2) sequences in which up to 9 amino acids of SEQ ID NO: 33 are substituted by other amino acids. In one embodiment, in such an ankyrin repeat domain said first ankyrin repeat module comprises an amino acid sequence selected from the group consisting of (1) SEQ ID NO: 32 and (2) sequences in which up to 6 amino acids in SEQ ID NO: 32 are substituted by other amino acids, and said second ankyrin repeat module comprises an amino acid sequence selected from the group consisting of (1) SEQ ID NO: 33 and (2) sequences in which up to 6 amino acids of SEQ ID NO: 33 are substituted by other amino acids. In one embodiment, in such an ankyrin repeat domain said first ankyrin repeat module comprises an amino acid sequence selected from the group consisting of (1) SEQ ID NO: 32 and (2) sequences in which up to 5 amino acids in SEQ ID NO: 32 are substituted by other amino acids, and said second ankyrin repeat module comprises an amino acid sequence selected from the group consisting of (1) SEQ ID NO: 33 and (2) sequences in which up to 5 amino acids of SEQ ID NO: 33 are substituted by other amino acids. In one embodiment, in such an ankyrin repeat domain said first ankyrin repeat module comprises an amino acid sequence selected from the group consisting of (1) SEQ ID NO: 32 and (2) sequences in which up to 4 amino acids in SEQ ID NO: 32 are substituted by other amino acids, and said second ankyrin repeat module comprises an amino acid sequence selected from the group consisting of (1) SEQ ID NO: 33 and (2) sequences in which up to 4 amino acids of SEQ ID NO: 33 are substituted by other amino acids. In one embodiment, in such an ankyrin repeat domain said first ankyrin repeat module comprises an amino acid sequence selected from the group consisting of (1) SEQ ID NO: 32 and (2) sequences in which up to 3 amino acids in SEQ ID NO: 32 are substituted by other amino acids, and said second ankyrin repeat module comprises an amino acid sequence selected from the group consisting of (1) SEQ ID NO: 33 and (2) sequences in which up to 3 amino acids of SEQ ID NO: 33 are substituted by other amino acids. In one embodiment, in such an ankyrin repeat domain said first ankyrin repeat module comprises an amino acid sequence selected from the group consisting of (1) SEQ ID NO: 32 and (2) sequences in which up to 2 amino acids in SEQ ID NO: 32 are substituted by other amino acids, and said second ankyrin repeat module comprises an amino acid sequence selected from the group consisting of (1) SEQ ID NO: 33 and (2) sequences in which up to 2 amino acids of SEQ ID NO: 33 are substituted by other amino acids. In one embodiment, in such an ankyrin repeat domain said first ankyrin repeat module comprises an amino acid sequence selected from the group consisting of (1) SEQ ID NO: 32 and (2) sequences in which 1 amino acid in SEQ ID NO: 32 is substituted by another amino acid, and said second ankyrin repeat module comprises an amino acid sequence selected from the group consisting of (1) SEQ ID NO: 33 and (2) sequences in which 1 amino acid of SEQ ID NO: 33 is substituted by another amino acid. In one embodiment, in such an ankyrin repeat domain said first ankyrin repeat module comprises the amino acid sequence of SEQ ID NO: 32 and said second ankyrin repeat module comprises the amino acid sequence of SEQ ID NO: 33.

In a preferred embodiment, said first ankyrin repeat module comprises an amino acid sequence selected from the group consisting of (1) SEQ ID NO: 32 and (2) sequences in which up to 3, up to 2 or up to 1 amino acids in SEQ ID NO: 32 are substituted by other amino acids, and said second ankyrin repeat module comprises an amino acid sequence selected from the group consisting of (1) SEQ ID NO: 33 and (2) sequences in which up to 3, up to 2 or up to 1 amino acids of SEQ ID NO: 33 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 32 and said second ankyrin repeat module comprises the amino acid sequence of SEQ ID NO: 33, wherein said first ankyrin repeat module is located N-terminally of said second ankyrin repeat module within said ankyrin repeat domain.

In another embodiment, said ankyrin repeat domain comprises a first ankyrin repeat module, a second ankyrin repeat module and a third ankyrin repeat module. In a preferred embodiment, said first, second and third ankyrin repeat modules are arranged as first ankyrin repeat module-second ankyrin repeat module-third ankyrin repeat module in N-terminal to C-terminal direction within said ankyrin repeat domain. In one embodiment, said first, second and third ankyrin repeat module each comprises an amino acid sequence selected from the group consisting of (1) SEQ ID NOs: 27 to 41 and (2) sequences in which up to 9, up to 8, up to 7, up to 6, up to 5, up to 4, up to 3, up to 2 or up to 1 amino acids in any of SEQ ID NOs: 27 to 41 are substituted by other amino acids. Thus, in one embodiment, said ankyrin repeat domain comprises a first ankyrin repeat module having an amino acid sequence selected from the group consisting of (1) SEQ ID NOs: 27 to 41 and (2) sequences in which up to 9 amino acids in any of SEQ ID NOs: 27 to 41 are substituted by other amino acids, further comprises a second ankyrin repeat module having an amino acid sequence selected from the group consisting of (1) SEQ ID NOs: 27 to 41 and (2) sequences in which up to 9 amino acids in any of SEQ ID NOs: 27 to 41 are substituted by other amino acids and further comprises a third ankyrin repeat module having an amino acid sequence selected from the group consisting of (1) SEQ ID NOs: 27 to 41 and (2) sequences in which up to 9 amino acids in any of SEQ ID NOs: 27 to 41 are substituted by other amino acids.

In one particular embodiment, in such an ankyrin repeat domain said first ankyrin repeat module comprises an amino acid sequence selected from the group consisting of (1) SEQ ID NO: 27 and (2) sequences in which up to 9, up to 8, up to 7, up to 6, up to 5, up to 4, up to 3, up to 2 or up to 1 amino acids in SEQ ID NO: 27 are substituted by other amino acids, said second ankyrin repeat module comprises an amino acid sequence selected from the group consisting of (1) SEQ ID NO: 28 and (2) sequences in which up to 9, up to 8, up to 7, up to 6, up to 5, up to 4, up to 3, up to 2 or up to 1 amino acids of SEQ ID NO: 28 are substituted by other amino acids and said third ankyrin repeat module comprises an amino acid sequence selected from the group consisting of (1) SEQ ID NO: 29 and (2) sequences in which up to 9, up to 8, up to 7, up to 6, up to 5, up to 4, up to 3, up to 2 or up to 1 amino acids in SEQ ID NO: 29 are substituted by other amino acids.

Thus, in one embodiment, in such an ankyrin repeat domain said first ankyrin repeat module comprises an amino acid sequence selected from the group consisting of (1) SEQ ID NO: 27 and (2) sequences in which up to 9 amino acids in SEQ ID NO: 27 are substituted by other amino acids, said second ankyrin repeat module comprises an amino acid sequence selected from the group consisting of (1) SEQ ID NO: 28 and (2) sequences in which up to 9 amino acids of SEQ ID NO: 28 are substituted by other amino acids and said third ankyrin repeat module comprises an amino acid sequence selected from the group consisting of (1) SEQ ID NO: 29 and (2) sequences in which up to 9 amino acids in SEQ ID NO: 29 are substituted by other amino acids.

in one embodiment, in such an ankyrin repeat domain said first ankyrin repeat module comprises an amino acid sequence selected from the group consisting of (1) SEQ ID NO: 27 and (2) sequences in which up to 5 amino acids in SEQ ID NO: 27 are substituted by other amino acids, said second ankyrin repeat module comprises an amino acid sequence selected from the group consisting of (1) SEQ ID NO: 28 and (2) sequences in which up to 5 amino acids of SEQ ID NO: 28 are substituted by other amino acids and said third ankyrin repeat module comprises an amino acid sequence selected from the group consisting of (1) SEQ ID NO: 29 and (2) sequences in which up to 5 amino acids in SEQ ID NO: 29 are substituted by other amino acids.

In one embodiment, in such an ankyrin repeat domain said first ankyrin repeat module comprises an amino acid sequence selected from the group consisting of (1) SEQ ID NO: 27 and (2) sequences in which up to 4 amino acids in SEQ ID NO: 27 are substituted by other amino acids, said second ankyrin repeat module comprises an amino acid sequence selected from the group consisting of (1) SEQ ID NO: 28 and (2) sequences in which up to 4 amino acids of SEQ ID NO: 28 are substituted by other amino acids and said third ankyrin repeat module comprises an amino acid sequence selected from the group consisting of (1) SEQ ID NO: 29 and (2) sequences in which up to 4 amino acids in SEQ ID NO: 29 are substituted by other amino acids.

In one embodiment, in such an ankyrin repeat domain said first ankyrin repeat module comprises an amino acid sequence selected from the group consisting of (1) SEQ ID NO: 27 and (2) sequences in which up to 3 amino acids in SEQ ID NO: 27 are substituted by other amino acids, said second ankyrin repeat module comprises an amino acid sequence selected from the group consisting of (1) SEQ ID NO: 28 and (2) sequences in which up to 3 amino acids of SEQ ID NO: 28 are substituted by other amino acids and said third ankyrin repeat module comprises an amino acid sequence selected from the group consisting of (1) SEQ ID NO: 29 and (2) sequences in which up to 3 amino acids in SEQ ID NO: 29 are substituted by other amino acids.

In one embodiment, in such an ankyrin repeat domain said first ankyrin repeat module comprises an amino acid sequence selected from the group consisting of (1) SEQ ID NO: 27 and (2) sequences in which up to 2 amino acids in SEQ ID NO: 27 are substituted by other amino acids, said second ankyrin repeat module comprises an amino acid sequence selected from the group consisting of (1) SEQ ID NO: 28 and (2) sequences in which up to 2 amino acids of SEQ ID NO: 28 are substituted by other amino acids and said third ankyrin repeat module comprises an amino acid sequence selected from the group consisting of (1) SEQ ID NO: 29 and (2) sequences in which up to 2 amino acids in SEQ ID NO: 29 are substituted by other amino acids.

In one embodiment, in such an ankyrin repeat domain said first ankyrin repeat module comprises an amino acid sequence selected from the group consisting of (1) SEQ ID NO: 27 and (2) sequences in which up to 1 amino acids in SEQ ID NO: 27 are substituted by other amino acids, said second ankyrin repeat module comprises an amino acid sequence selected from the group consisting of (1) SEQ ID NO: 28 and (2) sequences in which up to 1 amino acids of SEQ ID NO: 28 are substituted by other amino acids and said third ankyrin repeat module comprises an amino acid sequence selected from the group consisting of (1) SEQ ID NO: 29 and (2) sequences in which up to 1 amino acids in SEQ ID NO: 29 are substituted by other amino acids.

In one embodiment, in such an ankyrin repeat domain said first ankyrin repeat module comprises the amino acid sequence of SEQ ID NO: 27, said second ankyrin repeat module comprises the amino acid sequence of SEQ ID NO: 28 and said third ankyrin repeat module comprises the amino acid sequence of SEQ ID NO: 29.

In a preferred embodiment, said first ankyrin repeat module comprises an amino acid sequence selected from the group consisting of (1) SEQ ID NO: 27 and (2) sequences in which up to 3, up to 2 or up to 1 amino acids in SEQ ID NO: 27 are substituted by other amino acids, said second ankyrin repeat module comprises an amino acid sequence selected from the group consisting of (1) SEQ ID NO: 28 and (2) sequences in which up to 3, up to 2 or up to 1 amino acids of SEQ ID NO: 28 are substituted by other amino acids, and said third ankyrin repeat module comprises an amino acid sequence selected from the group consisting of (1) SEQ ID NO: 29 and (2) sequences in which up to 3, up to 2 or up to 1 amino acids of SEQ ID NO: 29 are substituted by other amino acids, wherein said first, second and third ankyrin repeat modules are arranged as first ankyrin repeat module-second ankyrin repeat module-third ankyrin repeat module in N-terminal to C-terminal direction within said ankyrin repeat domain.

Further, in a more preferred embodiment, said first ankyrin repeat module comprises the amino acid sequence of SEQ ID NO: 27, said second ankyrin repeat module comprises the amino acid sequence of SEQ ID NO: 28, and said third ankyrin repeat module comprises the amino acid sequence of SEQ ID NO: 29, wherein said first, second and third ankyrin repeat modules are arranged as first ankyrin repeat module-second ankyrin repeat module-third ankyrin repeat module in N-terminal to C-terminal direction within said ankyrin repeat domain.

In one particular embodiment, in such an ankyrin repeat domain said first ankyrin repeat module comprises an amino acid sequence selected from the group consisting of (1) SEQ ID NO: 32 and (2) sequences in which up to 9, up to 8, up to 7, up to 6, up to 5, up to 4, up to 3, up to 2 or up to 1 amino acids in SEQ ID NO: 32 are substituted by other amino acids, said second ankyrin repeat module comprises an amino acid sequence selected from the group consisting of (1) SEQ ID NO: 33 and (2) sequences in which up to 9, up to 8, up to 7, up to 6, up to 5, up to 4, up to 3, up to 2 or up to 1 amino acids of SEQ ID NO: 33 are substituted by other amino acids and said third ankyrin repeat module comprises an amino acid sequence selected from the group consisting of (1) SEQ ID NO: 34 and (2) sequences in which up to 9, up to 8, up to 7, up to 6, up to 5, up to 4, up to 3, up to 2 or up to 1 amino acids in SEQ ID NO: 34 are substituted by other amino acids.

Thus, in one embodiment, in such an ankyrin repeat domain said first ankyrin repeat module comprises an amino acid sequence selected from the group consisting of (1) SEQ ID NO: 32 and (2) sequences in which up to 9 amino acids in SEQ ID NO: 32 are substituted by other amino acids, said second ankyrin repeat module comprises an amino acid sequence selected from the group consisting of (1) SEQ ID NO: 33 and (2) sequences in which up to 9 amino acids of SEQ ID NO: 33 are substituted by other amino acids and said third ankyrin repeat module comprises an amino acid sequence selected from the group consisting of (1) SEQ ID NO: 34 and (2) sequences in which up to 9 amino acids in SEQ ID NO: 34 are substituted by other amino acids.

In one embodiment, in such an ankyrin repeat domain said first ankyrin repeat module comprises an amino acid sequence selected from the group consisting of (1) SEQ ID NO: 32 and (2) sequences in which up to 5 amino acids in SEQ ID NO: 32 are substituted by other amino acids, said second ankyrin repeat module comprises an amino acid sequence selected from the group consisting of (1) SEQ ID NO: 33 and (2) sequences in which up to 5 amino acids of SEQ ID NO: 33 are substituted by other amino acids and said third ankyrin repeat module comprises an amino acid sequence selected from the group consisting of (1) SEQ ID NO: 34 and (2) sequences in which up to 5 amino acids in SEQ ID NO: 34 are substituted by other amino acids.

In one embodiment, in such an ankyrin repeat domain said first ankyrin repeat module comprises an amino acid sequence selected from the group consisting of (1) SEQ ID NO: 32 and (2) sequences in which up to 4 amino acids in SEQ ID NO: 32 are substituted by other amino acids, said second ankyrin repeat module comprises an amino acid sequence selected from the group consisting of (1) SEQ ID NO: 33 and (2) sequences in which up to 4 amino acids of SEQ ID NO: 33 are substituted by other amino acids and said third ankyrin repeat module comprises an amino acid sequence selected from the group consisting of (1) SEQ ID NO: 34 and (2) sequences in which up to 4 amino acids in SEQ ID NO: 34 are substituted by other amino acids.

In one embodiment, in such an ankyrin repeat domain said first ankyrin repeat module comprises an amino acid sequence selected from the group consisting of (1) SEQ ID NO: 32 and (2) sequences in which up to 3 amino acids in SEQ ID NO: 32 are substituted by other amino acids, said second ankyrin repeat module comprises an amino acid sequence selected from the group consisting of (1) SEQ ID NO: 33 and (2) sequences in which up to 3 amino acids of SEQ ID NO: 33 are substituted by other amino acids and said third ankyrin repeat module comprises an amino acid sequence selected from the group consisting of (1) SEQ ID NO: 34 and (2) sequences in which up to 3 amino acids in SEQ ID NO: 34 are substituted by other amino acids.

In one embodiment, in such an ankyrin repeat domain said first ankyrin repeat module comprises an amino acid sequence selected from the group consisting of (1) SEQ ID NO: 32 and (2) sequences in which up to 2 amino acids in SEQ ID NO: 32 are substituted by other amino acids, said second ankyrin repeat module comprises an amino acid sequence selected from the group consisting of (1) SEQ ID NO: 33 and (2) sequences in which up to 2 amino acids of SEQ ID NO: 33 are substituted by other amino acids and said third ankyrin repeat module comprises an amino acid sequence selected from the group consisting of (1) SEQ ID NO: 34 and (2) sequences in which up to 2 amino acids in SEQ ID NO: 34 are substituted by other amino acids.

In one embodiment, in such an ankyrin repeat domain said first ankyrin repeat module comprises an amino acid sequence selected from the group consisting of (1) SEQ ID NO: 32 and (2) sequences in which up to 1 amino acids in SEQ ID NO: 32 are substituted by other amino acids, said second ankyrin repeat module comprises an amino acid sequence selected from the group consisting of (1) SEQ ID NO: 33 and (2) sequences in which up to 1 amino acids of SEQ ID NO: 33 are substituted by other amino acids and said third ankyrin repeat module comprises an amino acid sequence selected from the group consisting of (1) SEQ ID NO: 34 and (2) sequences in which up to 1 amino acids in SEQ ID NO: 34 are substituted by other amino acids.

In one embodiment, in such an ankyrin repeat domain said first ankyrin repeat module comprises the amino acid sequence of SEQ ID NO: 32, said second ankyrin repeat module comprises the amino acid sequence of SEQ ID NO: 33 and said third ankyrin repeat module comprises the amino acid sequence of SEQ ID NO: 34.

In a preferred embodiment, said first ankyrin repeat module comprises an amino acid sequence selected from the group consisting of (1) SEQ ID NO: 32 and (2) sequences in which up to 3, up to 2 or up to 1 amino acids in SEQ ID NO: 32 are substituted by other amino acids, said second ankyrin repeat module comprises an amino acid sequence selected from the group consisting of (1) SEQ ID NO: 33 and (2) sequences in which up to 3, up to 2 or up to 1 amino acids of SEQ ID NO: 33 are substituted by other amino acids, and said third ankyrin repeat module comprises an amino acid sequence selected from the group consisting of (1) SEQ ID NO: 34 and (2) sequences in which up to 3, up to 2 or up to 1 amino acids of SEQ ID NO: 34 are substituted by other amino acids, wherein said first, second and third ankyrin repeat modules are arranged as first ankyrin repeat module-second ankyrin repeat module-third ankyrin repeat module in N-terminal to C-terminal direction within said ankyrin repeat domain.

Further, in a more preferred embodiment, said first ankyrin repeat module comprises the amino acid sequence of SEQ ID NO: 32, said second ankyrin repeat module comprises the amino acid sequence of SEQ ID NO: 33, and said third ankyrin repeat module comprises the amino acid sequence of SEQ ID NO: 34, wherein said first, second and third ankyrin repeat modules are arranged as first ankyrin repeat module-second ankyrin repeat module-third ankyrin repeat module in N-terminal to C-terminal direction within said ankyrin repeat domain.

In further embodiments, said first, second or third ankyrin repeat module comprised in said ankyrin repeat domain independently comprises a sequence selected from the group consisting of (1) SEQ ID NOs: 52 to 57 and 97 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any one of SEQ ID NOs: 52 to 57 and 97 are substituted by another amino acid.

The ankyrin repeat domains of the recombinant binding protein disclosed herein preferably comprise a N-terminal and/or a C-terminal capping module (thereafter also referred to as capping repeats). Capping modules are located at the N- and/or C-terminal end of an ankyrin repeat domain, typically forming tight tertiary interactions (i.e. tertiary structure interactions) with the internal ankyrin repeat module(s), thereby providing a cap that shields the hydrophobic core of the ankyrin repeat domain at the side from exposure to the solvent. Examples of capping sequences are described in International Patent Publication Nos. WO 2002/020565 and WO 2012/069655, in U.S. Patent Publication No. US20130296221, and by Interlandi et al., J Mol Biol. 2008 Jan. 18; 375(3):837-54. Examples of N-terminal capping modules (i.e. N-terminal capping repeats) are provided with SEQ ID NOs: 42 to 46 and 58 to 61 and examples of C-terminal capping modules (i.e. C-terminal capping repeats) are provided with SEQ ID NOs: 47 to 51 and 62 to 68.

Accordingly, in some embodiments, the invention provides a recombinant binding protein comprising an ankyrin repeat domain as described herein, wherein said ankyrin repeat domain has binding specificity for CD16a and wherein said ankyrin repeat domain comprises an N-terminal capping module having an amino acid sequence selected from the group consisting of (1) SEQ ID NOs: 42 to 46 and (2) sequences in which up to 9, up to 8, up to 7, up to 6, up to 5, up to 4, up to 3, up to 2 or up to 1 amino acid(s) in any of SEQ ID NOs: 42 to 46 are substituted by other amino acids. Alternatively or in addition, said ankyrin repeat domain comprises an C-terminal capping module having an amino acid sequence selected from the group consisting of (1) SEQ ID NOs: 47 to 51 and (2) sequences in which up to 9, up to 8, up to 7, up to 6, up to 5, up to 4, up to 3, up to 2 or up to 1 amino acid(s) in any of SEQ ID NOs: 47 to 51 are substituted by other amino acids.

In further embodiments, the N-terminal capping module comprised in said ankyrin repeat domain comprises a sequence selected from the group consisting of (1) SEQ ID NOs: 58 to 61 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any one of SEQ ID NOs: 58 to 61 are substituted by another amino acid. Alternatively or in addition, the C-terminal capping module comprised in said designed ankyrin repeat domain comprises a sequence selected from the group consisting of (1) SEQ ID NOs: 62 to 68 and (2) sequences in which up to 9 framework residues, up to 8 framework residues, up to 7 framework residues, up to 6 framework residues, up to 5 framework residues, up to 4 framework residues, up to 3 framework residues, up to 2 framework residues, or up to 1 framework residue in any one of SEQ ID NOs: 62 to 68 are substituted by another amino acid.

In one preferred embodiment, all of said amino acid substitutions of said ankyrin repeat module(s) as described and referred to herein occur in framework positions of said ankyrin repeat module(s), wherein typically the overall structure of the module(s) is not affected by the substitutions. Such an embodiment of substitution in framework positions shall apply to all embodiments irrespective of whether such substitution is explicitly described. For clarity, the term “ankyrin repeat module” includes N-terminal and C-terminal capping modules.

In one preferred embodiment, all of said amino acid substitutions of said ankyrin repeat module(s) as described and referred to herein occur in framework positions and in positions other than positions 3, 4, 6, 14 and 15, preferably other than positions 3, 4, 5, 6, 11, 14 and 15 of said ankyrin repeat module(s) of SEQ ID NOs: 27 to 41 and 52 to 57 and 97, in positions other than positions 4, 8, 11 and 12 of SEQ ID NOs: 42 to 46 and 58 to 61, and in positions other than positions 3, 4, 6, 14 and 15 of SEQ ID NOs: 47 to 51 and 62 to 68, wherein typically the overall structure of the module(s) is not affected by the substitutions.

In another aspect, the invention relates to a recombinant binding protein comprising an ankyrin repeat domain, wherein said ankyrin repeat domain has binding specificity for CD16a, wherein said ankyrin repeat domain comprises an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% amino acid sequence identity with any one of SEQ ID NOs: 1 to 7, and wherein A at the second last position of SEQ ID NOs: 1 to 7 is optionally substituted by L, and/or A at the last position of SEQ ID NOs: 1 to 7 is optionally substituted by N.

Thus, in one embodiment, said ankyrin repeat domain comprises an amino acid sequence with at least 80% amino acid sequence identity with any one of SEQ ID NOs: 1 to 7. In one embodiment, said ankyrin repeat domain comprises an amino acid sequence with at least 90% amino acid sequence identity with any one of SEQ ID NOs: 1 to 7. In another embodiment, said ankyrin repeat domain comprises an amino acid sequence with at least 93% amino acid sequence identity with any one of SEQ ID NOs: 1 to 7. In a further embodiment, said ankyrin repeat domain comprises an amino acid sequence with at least 95% amino acid sequence identity with any one of SEQ ID NOs: 1 to 7. In one embodiment, said ankyrin repeat domain comprises an amino acid sequence with at least 98% amino acid sequence identity with any one of SEQ ID NOs: 1 to 7. In one embodiment, said ankyrin repeat domain comprises an amino acid sequence of any one of SEQ ID NOs: 1 to 7. In one embodiment, said recombinant binding protein comprises an ankyrin repeat domain with binding specificity for CD16a, wherein said ankyrin repeat domain comprises an amino acid sequence selected from SEQ ID NOs: 1 to 7, and wherein A at the second last position of SEQ ID NOs: 1 to 7 is optionally substituted by L, and/or A at the last position of SEQ ID NOs: 1 to 7 is optionally substituted by N.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 1, and wherein A at the second last position of SEQ ID NO: 1 is optionally substituted by L, and/or A at the last position of SEQ ID NO: 1 is optionally substituted by N. Thus, in one embodiment, said ankyrin repeat domain comprises an amino acid sequence with at least 80% amino acid sequence identity with SEQ ID NO: 1. In one embodiment, said ankyrin repeat domain comprises an amino acid sequence with at least 90% amino acid sequence identity with SEQ ID NO: 1. In another embodiment, said ankyrin repeat domain comprises an amino acid sequence with at least 93% amino acid sequence identity with SEQ ID NO: 1; and in a further embodiment, said ankyrin repeat domain comprises an amino acid sequence with at least 95% amino acid sequence identity with SEQ ID NO: 1. In one embodiment, said ankyrin repeat domain comprises an amino acid sequence with at least 98% amino acid sequence identity with SEQ ID NO: 1; and in one embodiment, said ankyrin repeat domain comprises the amino acid sequence of SEQ ID NO: 1. Thus, in one embodiment, said recombinant binding protein comprises an ankyrin repeat domain having binding specificity for CD16a, wherein said ankyrin repeat domain comprises the amino acid sequence of SEQ ID NO: 1.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 2, and wherein A at the second last position of SEQ ID NO: 2 is optionally substituted by L, and/or A at the last position of SEQ ID NO: 2 is optionally substituted by N. Thus, in one embodiment, said ankyrin repeat domain comprises an amino acid sequence with at least 80% amino acid sequence identity with SEQ ID NO: 2. In one embodiment, said ankyrin repeat domain comprises an amino acid sequence with at least 90% amino acid sequence identity with SEQ ID NO: 2. In another embodiment, said ankyrin repeat domain comprises an amino acid sequence with at least 93% amino acid sequence identity with SEQ ID NO: 2; and in a further embodiment, said ankyrin repeat domain comprises an amino acid sequence with at least 95% amino acid sequence identity with SEQ ID NO: 2. In one embodiment, said ankyrin repeat domain comprises an amino acid sequence with at least 98% amino acid sequence identity with SEQ ID NO: 2; and in one embodiment, said ankyrin repeat domain comprises the amino acid sequence of SEQ ID NO: 2. Thus, in one embodiment, said recombinant binding protein comprises an ankyrin repeat domain having binding specificity for CD16a, wherein said ankyrin repeat domain comprises the amino acid sequence of SEQ ID NO: 2.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 3, and wherein A at the second last position of SEQ ID NO: 3 is optionally substituted by L, and/or A at the last position of SEQ ID NO: 3 is optionally substituted by N. Thus, in one embodiment, said ankyrin repeat domain comprises an amino acid sequence with at least 80% amino acid sequence identity with SEQ ID NO: 3. In one embodiment, said ankyrin repeat domain comprises an amino acid sequence with at least 90% amino acid sequence identity with SEQ ID NO: 3. In another embodiment, said ankyrin repeat domain comprises an amino acid sequence with at least 93% amino acid sequence identity with SEQ ID NO: 3; and in a further embodiment, said ankyrin repeat domain comprises an amino acid sequence with at least 95% amino acid sequence identity with SEQ ID NO: 3. In one embodiment, said ankyrin repeat domain comprises an amino acid sequence with at least 98% amino acid sequence identity with SEQ ID NO: 3; and in one embodiment, said ankyrin repeat domain comprises the amino acid sequence of SEQ ID NO: 3. Thus, in one embodiment, said recombinant binding protein comprises an ankyrin repeat domain having binding specificity for CD16a, wherein said ankyrin repeat domain comprises the amino acid sequence of SEQ ID NO: 3.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 4, and wherein A at the second last position of SEQ ID NO: 4 is optionally substituted by L, and/or A at the last position of SEQ ID NO: 4 is optionally substituted by N. Thus, in one embodiment, said ankyrin repeat domain comprises an amino acid sequence with at least 80% amino acid sequence identity with SEQ ID NO: 4. In one embodiment, said ankyrin repeat domain comprises an amino acid sequence with at least 90% amino acid sequence identity with SEQ ID NO: 4. In another embodiment, said ankyrin repeat domain comprises an amino acid sequence with at least 93% amino acid sequence identity with SEQ ID NO: 4; and in a further embodiment, said ankyrin repeat domain comprises an amino acid sequence with at least 95% amino acid sequence identity with SEQ ID NO: 4. In one embodiment, said ankyrin repeat domain comprises an amino acid sequence with at least 98% amino acid sequence identity with SEQ ID NO: 4; and in one embodiment, said ankyrin repeat domain comprises the amino acid sequence of SEQ ID NO: 4. Thus, in one embodiment, said recombinant binding protein comprises an ankyrin repeat domain having binding specificity for CD16a, wherein said ankyrin repeat domain comprises the amino acid sequence of SEQ ID NO: 4.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 5, and wherein A at the second last position of SEQ ID NO: 5 is optionally substituted by L, and/or A at the last position of SEQ ID NO: 5 is optionally substituted by N. Thus, in one embodiment, said ankyrin repeat domain comprises an amino acid sequence with at least 80% amino acid sequence identity with SEQ ID NO: 5. In one embodiment, said ankyrin repeat domain comprises an amino acid sequence with at least 90% amino acid sequence identity with SEQ ID NO: 5. In another embodiment, said ankyrin repeat domain comprises an amino acid sequence with at least 93% amino acid sequence identity with SEQ ID NO: 5; and in a further embodiment, said ankyrin repeat domain comprises an amino acid sequence with at least 95% amino acid sequence identity with SEQ ID NO: 5. In one embodiment, said ankyrin repeat domain comprises an amino acid sequence with at least 98% amino acid sequence identity with SEQ ID NO: 5; and in one embodiment, said ankyrin repeat domain comprises the amino acid sequence of SEQ ID NO: 5. Thus, in one embodiment, said recombinant binding protein comprises an ankyrin repeat domain having binding specificity for CD16a, wherein said ankyrin repeat domain comprises the amino acid sequence of SEQ ID NO: 5.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 6, and wherein A at the second last position of SEQ ID NO: 6 is optionally substituted by L, and/or A at the last position of SEQ ID NO: 6 is optionally substituted by N. Thus, in one embodiment, said ankyrin repeat domain comprises an amino acid sequence with at least 80% amino acid sequence identity with SEQ ID NO: 6. In one embodiment, said ankyrin repeat domain comprises an amino acid sequence with at least 90% amino acid sequence identity with SEQ ID NO: 6. In another embodiment, said ankyrin repeat domain comprises an amino acid sequence with at least 93% amino acid sequence identity with SEQ ID NO: 6; and in a further embodiment, said ankyrin repeat domain comprises an amino acid sequence with at least 95% amino acid sequence identity with SEQ ID NO: 6. In one embodiment, said ankyrin repeat domain comprises an amino acid sequence with at least 98% amino acid sequence identity with SEQ ID NO: 6; and in one embodiment, said ankyrin repeat domain comprises the amino acid sequence of SEQ ID NO: 6. Thus, in one embodiment, said recombinant binding protein comprises an ankyrin repeat domain having binding specificity for CD16a, wherein said ankyrin repeat domain comprises the amino acid sequence of SEQ ID NO: 6.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 7, and wherein A at the second last position of SEQ ID NO: 7 is optionally substituted by L, and/or A at the last position of SEQ ID NO: 7 is optionally substituted by N. Thus, in one embodiment, said ankyrin repeat domain comprises an amino acid sequence with at least 80% amino acid sequence identity with SEQ ID NO: 7. In one embodiment, said ankyrin repeat domain comprises an amino acid sequence with at least 90% amino acid sequence identity with SEQ ID NO: 7. In another embodiment, said ankyrin repeat domain comprises an amino acid sequence with at least 93% amino acid sequence identity with SEQ ID NO: 7; and in a further embodiment, said ankyrin repeat domain comprises an amino acid sequence with at least 95% amino acid sequence identity with SEQ ID NO: 7. In one embodiment, said ankyrin repeat domain comprises an amino acid sequence with at least 98% amino acid sequence identity with SEQ ID NO: 7; and in one embodiment, said ankyrin repeat domain comprises the amino acid sequence of SEQ ID NO: 7. Thus, in one embodiment, said recombinant binding protein comprises an ankyrin repeat domain having binding specificity for CD16a, wherein said ankyrin repeat domain comprises the amino acid sequence of SEQ ID NO: 7.

In one embodiment, said recombinant binding protein comprises an ankyrin repeat domain with binding specificity for CD16a, wherein the potential interaction residues in said ankyrin repeat domain are identical to the corresponding positions in any one of the ankyrin repeat domains of SEQ ID NOs: 1 to 7.

In one embodiment, the recombinant binding protein comprising an ankyrin repeat domain with binding specificity for CD16a according to the invention binds to human CD16a with a dissociation constant (K_{D}) of or below about 10⁻⁷M, or of or below about 10⁻⁸M, or of or below about 10⁻⁹M. Thus, in one embodiment, said recombinant binding protein binds to CD16a with a K_{D} of or below about 10⁻⁷M. In another embodiment, said recombinant binding protein binds to CD16a with a K_{D} of or below about 10⁻⁸M. In another embodiment, said recombinant binding protein binds to CD16a with a K_{D} of or below about 10⁻⁹M. Preferably, said binding is measured in PBS. In one embodiment, said binding is measured in PBS using surface plasmon resonance (SPR). In one embodiment, said binding is measured in PBS using surface plasmon resonance (SPR) as described in Example 6.

Accordingly, in one embodiment, said recombinant binding protein comprises an ankyrin repeat domain with binding specificity for CD16a, wherein said binding protein binds human CD16a in PBS with a K_{D} of or below about 10⁻⁷M, or of or below about 10⁻⁸M, or of or below about 10⁻⁹M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 7, wherein A at the second last position of SEQ ID NOs: 1 to 7 is optionally substituted by L, and/or A at the last position of SEQ ID NOs: 1 to 7 is optionally substituted by N.

In one embodiment, said recombinant binding protein comprises an ankyrin repeat domain having binding specificity for CD16a, wherein said binding protein binds human CD16a in PBS with a K_{D} of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with of SEQ ID NO: 1, and wherein A at the second last position of SEQ ID NO: 1 is optionally substituted by L, and/or A at the last position of SEQ ID NO: 1 is optionally substituted by N. Thus, in one embodiment, said binding protein binds human CD16a in PBS with a K_{D} of or below about 10⁻⁸M, and said ankyrin repeat domain comprises an amino acid sequence with at least 80% amino acid sequence identity with the SEQ ID NO: 1. In one embodiment, said binding protein binds human CD16a in PBS with a K_{D} below 10⁻⁸M, and said ankyrin repeat domain comprises an amino acid sequence with at least 90% amino acid sequence identity with the SEQ ID NO: 1. In another embodiment, said binding protein binds human CD16a in PBS with a K_{D} below 10⁻⁸M, and said ankyrin repeat domain comprises an amino acid sequence with at least 93% amino acid sequence identity with SEQ ID NO: 1; and in a further embodiment, said binding protein binds human CD16a in PBS with a K_{D} below 10⁻⁸M, and said ankyrin repeat domain comprises an amino acid sequence with at least 95% amino acid sequence identity with SEQ ID NO: 1. In one embodiment, said binding protein binds human CD16a in PBS with a K_{D} below 10⁻⁸M, and said ankyrin repeat domain comprises an amino acid sequence with at least 98% amino acid sequence identity with SEQ ID NO: 1; and in one embodiment, said binding protein binds human CD16a in PBS with a K_{D} below 10⁻⁸M, and said ankyrin repeat domain comprises the amino acid sequence of SEQ ID NO: 1. Thus, in one embodiment, said recombinant binding protein comprises an ankyrin repeat domain with binding specificity for CD16a, wherein said binding protein binds human CD16a in PBS with a K_{D} of or below about 10⁻⁸M, and wherein said ankyrin repeat domain comprises the amino acid sequence of SEQ ID NO: 1, and wherein A at the second last position of SEQ ID NO: 1 is optionally substituted by L, and/or A at the last position of SEQ ID NO: 1 is optionally substituted by N.

In one embodiment, said recombinant binding protein comprises an ankyrin repeat domain having binding specificity for CD16a, wherein said binding protein binds human CD16a in PBS with a K_{D} of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with of SEQ ID NO: 2, and wherein A at the second last position of SEQ ID NO: 2 is optionally substituted by L, and/or A at the last position of SEQ ID NO: 2 is optionally substituted by N. Thus, in one embodiment, said binding protein binds human CD16a in PBS with a K_{D} of or below about 10⁻⁸M, and said ankyrin repeat domain comprises an amino acid sequence with at least 80% amino acid sequence identity with the SEQ ID NO: 2. In one embodiment, said binding protein binds human CD16a in PBS with a K_{D} below 10⁻⁸M, and said ankyrin repeat domain comprises an amino acid sequence with at least 90% amino acid sequence identity with the SEQ ID NO: 2. In another embodiment, said binding protein binds human CD16a in PBS with a K_{D} below 10⁻⁸M, and said ankyrin repeat domain comprises an amino acid sequence with at least 93% amino acid sequence identity with SEQ ID NO: 2; and in a further embodiment, said binding protein binds human CD16a in PBS with a K_{D} below 10⁻⁸M, and said ankyrin repeat domain comprises an amino acid sequence with at least 95% amino acid sequence identity with SEQ ID NO: 2. In one embodiment, said binding protein binds human CD16a in PBS with a K_{D} below 10⁻⁸M, and said ankyrin repeat domain comprises an amino acid sequence with at least 98% amino acid sequence identity with SEQ ID NO: 2; and in one embodiment, said binding protein binds human CD16a in PBS with a K_{D} below 10⁻⁸M, and said ankyrin repeat domain comprises the amino acid sequence of SEQ ID NO: 2. Thus, in one embodiment, said recombinant binding protein comprises an ankyrin repeat domain with binding specificity for CD16a, wherein said binding protein binds human CD16a in PBS with a K_{D} of or below about 10⁻⁸M, and wherein said ankyrin repeat domain comprises the amino acid sequence of SEQ ID NO: 2, and wherein A at the second last position of SEQ ID NO: 2 is optionally substituted by L, and/or A at the last position of SEQ ID NO: 2 is optionally substituted by N.

In one embodiment, said recombinant binding protein comprises an ankyrin repeat domain having binding specificity for CD16a, wherein said binding protein binds human CD16a in PBS with a K_{D} of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with of SEQ ID NO: 3, and wherein A at the second last position of SEQ ID NO: 3 is optionally substituted by L, and/or A at the last position of SEQ ID NO: 3 is optionally substituted by N. Thus, in one embodiment, said binding protein binds human CD16a in PBS with a K_{D} of or below about 10⁻⁸M, and said ankyrin repeat domain comprises an amino acid sequence with at least 80% amino acid sequence identity with the SEQ ID NO: 3. In one embodiment, said binding protein binds human CD16a in PBS with a K_{D} below 10⁻⁸M, and said ankyrin repeat domain comprises an amino acid sequence with at least 90% amino acid sequence identity with the SEQ ID NO: 3. In another embodiment, said binding protein binds human CD16a in PBS with a K_{D} below 10⁻⁸M, and said ankyrin repeat domain comprises an amino acid sequence with at least 93% amino acid sequence identity with SEQ ID NO: 3; and in a further embodiment, said binding protein binds human CD16a in PBS with a K_{D} below 10⁻⁸M, and said ankyrin repeat domain comprises an amino acid sequence with at least 95% amino acid sequence identity with SEQ ID NO: 3. In one embodiment, said binding protein binds human CD16a in PBS with a K_{D} below 10⁻⁸M, and said ankyrin repeat domain comprises an amino acid sequence with at least 98% amino acid sequence identity with SEQ ID NO: 3; and in one embodiment, said binding protein binds human CD16a in PBS with a K_{D} below 10⁻⁸M, and said ankyrin repeat domain comprises the amino acid sequence of SEQ ID NO: 3. Thus, in one embodiment, said recombinant binding protein comprises an ankyrin repeat domain with binding specificity for CD16a, wherein said binding protein binds human CD16a in PBS with a K_{D} of or below about 10⁻⁸M, and wherein said ankyrin repeat domain comprises the amino acid sequence of SEQ ID NO: 3, and wherein A at the second last position of SEQ ID NO: 3 is optionally substituted by L, and/or A at the last position of SEQ ID NO: 3 is optionally substituted by N.

In one embodiment, said recombinant binding protein comprises an ankyrin repeat domain having binding specificity for CD16a, wherein said binding protein binds human CD16a in PBS with a K_{D} of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with of SEQ ID NO: 4, and wherein A at the second last position of SEQ ID NO: 4 is optionally substituted by L, and/or A at the last position of SEQ ID NO: 4 is optionally substituted by N. Thus, in one embodiment, said binding protein binds human CD16a in PBS with a K_{D} of or below about 10⁻⁸M, and said ankyrin repeat domain comprises an amino acid sequence with at least 80% amino acid sequence identity with the SEQ ID NO: 4. In one embodiment, said binding protein binds human CD16a in PBS with a K_{D} below 10⁻⁸M, and said ankyrin repeat domain comprises an amino acid sequence with at least 90% amino acid sequence identity with the SEQ ID NO: 4. In another embodiment, said binding protein binds human CD16a in PBS with a K_{D} below 10⁻⁸M, and said ankyrin repeat domain comprises an amino acid sequence with at least 93% amino acid sequence identity with SEQ ID NO: 4; and in a further embodiment, said binding protein binds human CD16a in PBS with a K_{D} below 10⁻⁸M, and said ankyrin repeat domain comprises an amino acid sequence with at least 95% amino acid sequence identity with SEQ ID NO: 4. In one embodiment, said binding protein binds human CD16a in PBS with a K_{D} below 10⁻⁸M, and said ankyrin repeat domain comprises an amino acid sequence with at least 98% amino acid sequence identity with SEQ ID NO: 4; and in one embodiment, said binding protein binds human CD16a in PBS with a K_{D} below 10⁻⁸M, and said ankyrin repeat domain comprises the amino acid sequence of SEQ ID NO: 4. Thus, in one embodiment, said recombinant binding protein comprises an ankyrin repeat domain with binding specificity for CD16a, wherein said binding protein binds human CD16a in PBS with a K_{D} of or below about 10⁻⁸M, and wherein said ankyrin repeat domain comprises the amino acid sequence of SEQ ID NO: 4, and wherein A at the second last position of SEQ ID NO: 4 is optionally substituted by L, and/or A at the last position of SEQ ID NO: 4 is optionally substituted by N.

In one embodiment, said recombinant binding protein comprises an ankyrin repeat domain having binding specificity for CD16a, wherein said binding protein binds human CD16a in PBS with a K_{D} of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with of SEQ ID NO: 5, and wherein A at the second last position of SEQ ID NO: 5 is optionally substituted by L, and/or A at the last position of SEQ ID NO: 5 is optionally substituted by N. Thus, in one embodiment, said binding protein binds human CD16a in PBS with a K_{D} of or below about 10⁻⁸M, and said ankyrin repeat domain comprises an amino acid sequence with at least 80% amino acid sequence identity with the SEQ ID NO: 5. In one embodiment, said binding protein binds human CD16a in PBS with a K_{D} below 10⁻⁸M, and said ankyrin repeat domain comprises an amino acid sequence with at least 90% amino acid sequence identity with the SEQ ID NO: 5. In another embodiment, said binding protein binds human CD16a in PBS with a K_{D} below 10⁻⁸M, and said ankyrin repeat domain comprises an amino acid sequence with at least 93% amino acid sequence identity with SEQ ID NO: 5; and in a further embodiment, said binding protein binds human CD16a in PBS with a K_{D} below 10⁻⁸M, and said ankyrin repeat domain comprises an amino acid sequence with at least 95% amino acid sequence identity with SEQ ID NO: 5. In one embodiment, said binding protein binds human CD16a in PBS with a K_{D} below 10⁻⁸M, and said ankyrin repeat domain comprises an amino acid sequence with at least 98% amino acid sequence identity with SEQ ID NO: 5; and in one embodiment, said binding protein binds human CD16a in PBS with a K_{D} below 10⁻⁸M, and said ankyrin repeat domain comprises the amino acid sequence of SEQ ID NO: 5. Thus, in one embodiment, said recombinant binding protein comprises an ankyrin repeat domain with binding specificity for CD16a, wherein said binding protein binds human CD16a in PBS with a K_{D} of or below about 10⁻⁸M, and wherein said ankyrin repeat domain comprises the amino acid sequence of SEQ ID NO: 5, and wherein A at the second last position of SEQ ID NO: 5 is optionally substituted by L, and/or A at the last position of SEQ ID NO: 5 is optionally substituted by N.

In one embodiment, said recombinant binding protein comprises an ankyrin repeat domain having binding specificity for CD16a, wherein said binding protein binds human CD16a in PBS with a K_{D} of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with of SEQ ID NO: 6, and wherein A at the second last position of SEQ ID NO: 6 is optionally substituted by L, and/or A at the last position of SEQ ID NO: 6 is optionally substituted by N. Thus, in one embodiment, said binding protein binds human CD16a in PBS with a K_{D} of or below about 10⁻⁸M, and said ankyrin repeat domain comprises an amino acid sequence with at least 80% amino acid sequence identity with the SEQ ID NO: 6. In one embodiment, said binding protein binds human CD16a in PBS with a K_{D} below 10⁻⁸M, and said ankyrin repeat domain comprises an amino acid sequence with at least 90% amino acid sequence identity with the SEQ ID NO: 6. In another embodiment, said binding protein binds human CD16a in PBS with a K_{D} below 10⁻⁸M, and said ankyrin repeat domain comprises an amino acid sequence with at least 93% amino acid sequence identity with SEQ ID NO: 6; and in a further embodiment, said binding protein binds human CD16a in PBS with a K_{D} below 10⁻⁸M, and said ankyrin repeat domain comprises an amino acid sequence with at least 95% amino acid sequence identity with SEQ ID NO: 6. In one embodiment, said binding protein binds human CD16a in PBS with a K_{D} below 10⁻⁸M, and said ankyrin repeat domain comprises an amino acid sequence with at least 98% amino acid sequence identity with SEQ ID NO: 6; and in one embodiment, said binding protein binds human CD16a in PBS with a K_{D} below 10⁻⁸M, and said ankyrin repeat domain comprises the amino acid sequence of SEQ ID NO: 6. Thus, in one embodiment, said recombinant binding protein comprises an ankyrin repeat domain with binding specificity for CD16a, wherein said binding protein binds human CD16a in PBS with a K_{D} of or below about 10⁻⁸M, and wherein said ankyrin repeat domain comprises the amino acid sequence of SEQ ID NO: 6, and wherein A at the second last position of SEQ ID NO: 6 is optionally substituted by L, and/or A at the last position of SEQ ID NO: 6 is optionally substituted by N.

In one embodiment, said recombinant binding protein comprises an ankyrin repeat domain having binding specificity for CD16a, wherein said binding protein binds human CD16a in PBS with a K_{D} of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with of SEQ ID NO: 7, and wherein A at the second last position of SEQ ID NO: 7 is optionally substituted by L, and/or A at the last position of SEQ ID NO: 7 is optionally substituted by N. Thus, in one embodiment, said binding protein binds human CD16a in PBS with a K_{D} of or below about 10⁻⁸M, and said ankyrin repeat domain comprises an amino acid sequence with at least 80% amino acid sequence identity with the SEQ ID NO: 7. In one embodiment, said binding protein binds human CD16a in PBS with a K_{D} below 10⁻⁸M, and said ankyrin repeat domain comprises an amino acid sequence with at least 90% amino acid sequence identity with the SEQ ID NO: 7. In another embodiment, said binding protein binds human CD16a in PBS with a K_{D} below 10⁻⁸M, and said ankyrin repeat domain comprises an amino acid sequence with at least 93% amino acid sequence identity with SEQ ID NO: 7; and in a further embodiment, said binding protein binds human CD16a in PBS with a K_{D} below 10⁻⁸M, and said ankyrin repeat domain comprises an amino acid sequence with at least 95% amino acid sequence identity with SEQ ID NO: 7. In one embodiment, said binding protein binds human CD16a in PBS with a K_{D} below 10⁻⁸M, and said ankyrin repeat domain comprises an amino acid sequence with at least 98% amino acid sequence identity with SEQ ID NO: 7; and in one embodiment, said binding protein binds human CD16a in PBS with a K_{D} below 10⁻⁸M, and said ankyrin repeat domain comprises the amino acid sequence of SEQ ID NO: 7. Thus, in one embodiment, said recombinant binding protein comprises an ankyrin repeat domain with binding specificity for CD16a, wherein said binding protein binds human CD16a in PBS with a K_{D} of or below about 10⁻⁸M, and wherein said ankyrin repeat domain comprises the amino acid sequence of SEQ ID NO: 7, and wherein A at the second last position of SEQ ID NO: 7 is optionally substituted by L, and/or A at the last position of SEQ ID NO: 7 is optionally substituted by N.

In further embodiments, the recombinant binding protein comprising an ankyrin repeat domain with binding specificity for CD16a binds to human CD16a as described herein, and does not substantially bind to CD16b. Accordingly, in a preferred embodiment, the recombinant binding protein of the invention has a higher binding affinity for CD16a than to CD16b. Thus, in one embodiment the recombinant binding protein of the invention binds to CD16a with a K_{D} that is at least 2 times, at least 5 times, at least 10 times, at least 20 times, at least 50 times, at least 10² times, at least 10³ times, at least 10⁴ times or at least 10⁵ times lower than the K_{D} of said recombinant binding protein for CD16b.

In one embodiment, the invention relates to a recombinant binding protein comprising an ankyrin repeat domain with binding specificity for CD16a, wherein said ankyrin repeat domain has a melting temperature (Tm) higher than 65° C., higher than 68° C., higher than 70° C., higher than 72° C., higher than 75° C., higher than 78° C., higher than 80° C., of about 75° C., of about 80° C., of about 82° C., of about 85° C., of between 65° C. and 95° C., of between 70° C. and 90° C., or of between 72° C. and 88° C. Thus, in one embodiment, said ankyrin repeat domain has a melting temperature (Tm) higher than 65° C. In another embodiment, said ankyrin repeat domain has a melting temperature (Tm) higher than 70° C. In another embodiment, said ankyrin repeat domain has a melting temperature (Tm) higher than 75° C. In another embodiment, said ankyrin repeat domain has a melting temperature (Tm) higher than 80° C. In another embodiment, said ankyrin repeat domain has a melting temperature (Tm) of between 65° C. and 95° C. In another embodiment, said ankyrin repeat domain has a melting temperature (Tm) of between 72° C. and 88° C.

In one embodiment, the recombinant binding protein described herein comprises an ankyrin repeat domain with binding specificity for human CD16a and cynomolgus CD16a.

In a further aspect, the invention relates to the recombinant binding protein as described above, further comprising at least one binding moiety with binding specificity for a disease-associated antigen.

Thus, in a particular embodiment, the recombinant binding protein of the invention comprises an ankyrin repeat domain with binding specificity for CD16a as described herein and further comprises a binding moiety with binding specificity for a disease-associated antigen. In a preferred embodiment, said binding moiety with binding specificity for a disease-associated antigen is located N-terminally of said ankyrin repeat domain with binding specificity for CD16a within said binding protein. In another embodiment, said binding moiety with binding specificity for a disease-associated antigen is located C-terminally of said ankyrin repeat domain with binding specificity for CD16a within said binding protein.

In one preferred embodiment, said disease-associated antigen is a Tumor Associated Antigen (TAA). Illustrative examples of Tumor Associated Antigens include, but are not limited to, proteins expressed on the cell surface of tumor cells, such as, e.g., HER2, CD123, CD33, and CD70, or a peptide-MHC complex, wherein said peptide is derived from a protein expressed in a tumor cell (such as, e.g. NY-ESO-1 or MAGE-A3) and said MHC is MHC class I. In another embodiment, said disease-associated antigen is an Infection Associated Antigen, preferably a Virus Associated Antigen (VAA). Illustrative examples of Virus Associated Antigens include, but are not limited to, a peptide-MHC complex, wherein said peptide is derived from a viral infectious agent (such as, e.g. HBcAg or EBNA-1) and said MHC is MHC class I.

In one embodiment, said binding moiety with binding specificity for a disease-associated antigen is linked, conjugated, fused or otherwise physically attached to said ankyrin repeat domain with binding specificity for CD16a. In one embodiment, said binding moiety with binding specificity for a disease-associated antigen is covalently linked to said ankyrin repeat domain with binding specificity for CD16a. In one embodiment, said binding moiety with binding specificity for a disease-associated antigen is covalently linked to said ankyrin repeat domain with binding specificity for CD16a with a peptide linker. In one embodiment, the amino acid sequence of said peptide linker has a length from 1 to 50 amino acids, preferably from 6 to 38 amino acids. In one embodiment, said peptide linker is a proline-threonine rich peptide linker. In one embodiment, said binding moiety with binding specificity for a disease-associated antigen is covalently linked to said ankyrin repeat domain with binding specificity for CD16a with the proline-threonine rich peptide linker of SEQ ID NO: 70. In one preferred embodiment, said binding moiety with binding specificity for a disease-associated antigen is a designed ankyrin repeat domain with binding specificity for a disease-associated antigen, preferably a Tumor Associated Antigen (TAA). In further embodiments, each binding moiety with binding specificity for a TAA comprised in said recombinant binding protein of the invention is a designed ankyrin repeat domain with binding specificity for the respective TAA.

In another embodiment, the binding moiety with binding specificity for a disease-associated antigen is an antibody or a camelid nanobody. Camelid nanobodies (also known as camelid single-domain antibodies or VHHs) are derived from the Camelidae family of mammals such the llamas, camels, and alpacas. Unlike other antibodies, camelid antibodies lack a light chain and are composed of two identical heavy chains. Camelid antibodies typically have a relatively low molecular weight in the region of around 15 kDa. In one embodiment, the binding moiety with binding specificity for a disease-associated antigen is a shark antibody domain. Shark antibody domains, like camelid nanobodies, also lack a light chain.

In another embodiment, the binding moiety with binding specificity for a disease-associated antigen is an alternative scaffold. Alternative scaffolds include any polypeptides or proteins comprising a binding domain that is capable of binding an antigen (such as a drug molecule) and that is not derived from an antibody or immunoglobulin molecule. The binding domain of alternative scaffolds may comprise or may be derived from a variety of different polypeptide or protein structures. Alternative scaffolds include, but are not limited to, adnectins (monobodies), affibodies, affilins, affimers and aptamers, affitins, alphabodies, anticalins, armadillo repeat protein-based scaffolds, atrimers, avimers, ankyrin repeat protein-based scaffolds (such as DARPin proteins), fynomers, knottins, and Kunitz domain peptides. Alternative scaffolds are described, e.g., in Yu et al., Annu Rev Anal Chem (Palo Alto Calif). 2017 June 12; 10(1): 293-320. doi:10.1146/annurevanchem-061516-045205. Thus, in one embodiment, the binding moiety with binding specificity for a disease-associated antigen comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, or a Kunitz domain.

In one embodiment, the recombinant binding protein of the invention comprises an ankyrin repeat domain with binding specificity for CD16a as described herein and further comprises an ankyrin repeat domain with binding specificity for HER2. In one embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 10 to 21. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 10. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 11. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 12. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 13. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 14. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 15. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 16. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 17. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 18. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 19. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 20. In a particular embodiment, said recombinant protein comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 21.

In one embodiment, the recombinant binding protein of the invention comprises an ankyrin repeat domain with binding specificity for CD16a as described herein and further comprises an ankyrin repeat domain with binding specificity for CD47. Suitable ankyrin repeat domains with binding specificity for CD47 are described in EP23177836.

In one embodiment, the recombinant binding protein of the invention comprises an ankyrin repeat domain with binding specificity for CD16a as described herein and further comprises two ankyrin repeat domains with binding specificity for a disease-associated antigen. In preferred embodiments, said disease-associated antigen is a Tumor Associated Antigen (TAA).

In one embodiment, the recombinant binding protein of the invention comprises an ankyrin repeat domain with binding specificity for CD16a as described herein and further comprises an ankyrin repeat domain with dual binding specificity, wherein binding of said ankyrin repeat domain with dual binding specificity to its first and second binding targets is mutually exclusive. Such ankyrin repeat domains with dual binding specificity (also referred to as “2-in-1 repeat domains”) and corresponding production methods are described in PCT/EP2022/085794. In a more particular embodiment, the recombinant binding protein of the invention comprises an ankyrin repeat domain with binding specificity for CD16a as described herein and further comprises a second and a third ankyrin repeat domain, wherein the second ankyrin repeat domain has binding specificity for CD47 and the third ankyrin repeat domain is a 2-in-1 repeat domain having a first binding specificity for a TAA and a second binding specificity for said second ankyrin repeat domain having binding specificity for CD47. Such a design enables a conditional TAA-dependent activation of the CD47 binding domain, wherein upon presence of the TAA, the 2-in-1 repeat domain binds the TAA and releases the CD47-specific binding domain, thereby releasing the inhibition of the CD47-specific binding domain.

### Half-Life Extending Moieties

A “half-life extending moiety” extends the serum half-life in vivo of the recombinant binding proteins described herein, compared to the same protein without the half-life extending moiety. Examples of half-life extending moieties include, but are not limited to, polyhistidine, Glu-Glu, glutathione S transferase (GST), thioredoxin, protein A, protein G, an immunoglobulin domain, maltose binding protein (MBP), a human serum albumin (HSA) binding domain, or polyethylene glycol (PEG).

In one embodiment, the recombinant binding protein described herein further comprises one or more half-life extending moiety. Thus, in a particular embodiment, the recombinant binding protein of the invention comprises an ankyrin repeat domain with binding specificity for CD16a as described herein and further comprises one or more half-life extending moiety. Preferably, said half-life extending moiety binds to human serum albumin.

In some embodiments, said half-life extending moiety comprises a serum albumin-binding ankyrin repeat domain comprising an amino acid sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 24 to 26. In an exemplary embodiment, said half-life extending moiety comprises an amino acid sequence that is at least about 90% identical to any one of SEQ ID NOs: 24 to 26. In one embodiment, said half-life extending moiety comprises a serum albumin-binding ankyrin repeat domain comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 24. In an exemplary embodiment, said half-life extending moiety comprises a serum albumin-binding ankyrin repeat domain comprising an amino acid sequence that is at least 90% identical to SEQ ID NO: 24.

In some embodiments, said serum albumin binding domain is located at the N-terminus of the recombinant binding protein. In some embodiments, said serum albumin binding domain is located at the C-terminus of the recombinant binding protein. In some embodiments, two or more serum albumin binding ankyrin repeat domains are preferred. In some embodiments, two serum albumin binding ankyrin repeat domains are located at the N-terminus of the recombinant binding protein. In some embodiments, two serum albumin binding ankyrin repeat domains are located at the C-terminus of the recombinant binding protein. In some embodiments, a first serum albumin binding ankyrin repeat domains is located at the N-terminus of the recombinant binding protein, and a second serum albumin binding ankyrin repeat domains is located at the C-terminus of the recombinant binding protein.

In some embodiments, the half-life extending moiety comprises an immunoglobulin domain. In some embodiments, the immunoglobulin domain comprises an Fc domain. In some embodiments, the Fc domain is derived from any one of the known heavy chain isotypes: IgG (γ), IgM (μ), IgD (δ), IgE (ε), or IgA (α). In some embodiments, the Fc domain is derived from any one of the known heavy chain isotypes or subtypes: IgG₁ (γ1), IgG₂ (γ2), IgG₃ (γ3), IgG₄ (γ4), IgA₁ (α1), IgA₂ (α2). In some embodiments, the Fc domain is the Fc domain of human IgG₁.

In some embodiments, the Fc domain comprises an uninterrupted native sequence (i.e., wild type sequence) of an Fc domain. In some embodiments, the immunoglobulin Fc domain comprises a variant Fc domain resulting in altered biological activity. For example, at least one point mutation or deletion may be introduced into the Fc domain so as to reduce or eliminate the effector activity (e.g., International Patent Publication No. WO 2005/063815), and/or to increase the homogeneity during the production of the recombinant binding protein. In some embodiments, the Fc domain is the Fc domain of human IgG₁ and comprises one or more of the following effector-null substitutions: L234A, L235A, and G237A (Eu numbering). In some embodiments, the Fc domain does not comprise the lysine located at the C-terminal position of human IgG1 (i.e., K447 by Eu numbering). The absence of the lysine may increase homogeneity during the production of the recombinant binding protein. In some embodiments, the Fc domain comprises the lysine located at the C-terminal position (K447, Eu numbering).

### Substitutions

A substitution as recited in the embodiments of the present invention is preferably a conservative substitution according to Table A.

TABLE A
Amino Acid Substitutions

| Original | Conservative | |
|---|---|---|
| Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr(Y) | Phe | Trp; Phe; Thr; Ser |
| Val (V) | Leu | Ile; Leu; Met; Phe; Ala; Norleucine |

In addition, the second last position of any ankyrin repeat domain of a recombinant binding protein of the invention can be “A” or “L”, and/or the last position can be “A” or “N”. Furthermore, each ankyrin repeat domain comprised in a recombinant binding protein of the invention may optionally comprise a “G,” an “S,” or a “GS” sequence at its N-terminus.

### Nucleic Acids

In another aspect, the invention relates to a nucleic acid encoding the amino acid sequence of an ankyrin repeat domain or a recombinant binding protein of the present invention. In one embodiment, the invention relates to a nucleic acid encoding the amino acid sequence of a recombinant binding protein of the present invention. In one embodiment, the invention relates to a nucleic acid encoding an amino acid sequence selected from the group consisting of SEQ ID NO: 1 to 7. In one embodiment, the invention relates to a nucleic acid encoding the amino acid sequence of SEQ ID NO: 1. In one embodiment, the invention relates to a nucleic acid encoding the amino acid sequence of SEQ ID NO: 2. In one embodiment, the invention relates to a nucleic acid encoding the amino acid sequence of SEQ ID NO: 3. In one embodiment, the invention relates to a nucleic acid encoding the amino acid sequence of SEQ ID NO: 4. In one embodiment, the invention relates to a nucleic acid encoding the amino acid sequence of SEQ ID NO: 5. In one embodiment, the invention relates to a nucleic acid encoding the amino acid sequence of SEQ ID NO: 6. In one embodiment, the invention relates to a nucleic acid encoding the amino acid sequence of SEQ ID NO: 7. Furthermore, the invention relates to vectors comprising any nucleic acid of the invention. Nucleic acids are well known to the skilled person in the art. In the examples, nucleic acids were used to produce designed ankyrin repeat domains or recombinant binding proteins of the invention in E. coli. Examples of nucleic acids are provided by SEQ ID NOs: 77 to 83 which encode the amino acid sequences of SEQ ID NOs: 1 to 7, respectively.

### Compositions, Uses and Methods of Treatment

In one aspect, the invention relates to a pharmaceutical composition comprising a recombinant binding protein and/or a designed ankyrin repeat domain of the present invention, and/or a nucleic acid encoding a recombinant binding protein and/or a designed ankyrin repeat domain of the present invention, and optionally a pharmaceutically acceptable carrier and/or diluent.

In one aspect, the invention relates to a pharmaceutical composition comprising a recombinant binding protein or a nucleic acid encoding a recombinant binding protein of the present invention, and optionally a pharmaceutically acceptable carrier and/or diluent.

Pharmaceutically acceptable carriers and/or diluents are known to the person skilled in the art and are explained in more detail below.

A pharmaceutical composition comprises a recombinant binding protein, and/or a designed ankyrin repeat domain, and/or a nucleic acid, preferably a recombinant binding protein and/or a nucleic acid, as described herein and a pharmaceutically acceptable carrier, excipient, or stabilizer, for example as described in Remington's Pharmaceutical Sciences 16^{th} edition, Osol, A. Ed., 1980.

Suitable carriers, diluents, excipients or stabilizers known to one of skill in the art include, for example, saline, Ringer's solution, dextrose solution, Hank's solution, fixed oils, ethyl oleate, 5% dextrose in saline, substances that enhance isotonicity and chemical stability, buffers, and preservatives. Other suitable carriers include any carrier that does not itself induce the production of antibodies harmful to the individual receiving the composition such as proteins, polysaccharides, polylactic acids, polyglycolic acids, polymeric amino acids, and amino acid copolymers. A pharmaceutical composition may also be a combination formulation, comprising an additional active agent, such as an anti-cancer agent or an anti-angiogenic agent, or an additional bioactive compound. The compositions to be used for in vivo administration must be aseptic or sterile. This is readily accomplished by filtration through sterile filtration membranes.

In one aspect, a pharmaceutical composition comprises at least one recombinant binding protein as described herein and a detergent such as nonionic detergent, a buffer such as phosphate buffer, and a sugar such as sucrose. In one aspect, such a pharmaceutical composition comprises recombinant binding proteins as described above and PBS.

In another aspect, the invention provides a method of activating immune cells in a mammal, including a human, the method comprising the step of administering to said mammal the recombinant protein of the invention, the nucleic acid of the invention or the pharmaceutical composition of the invention. In one embodiment, said activation is tumor-localized. In one embodiment, said immune cells are innate immune cells. In a one embodiment, said innate immune cells are NK cells. In one embodiment, said innate immune cells are macrophages. In one embodiment, said innate immune cells comprise NK cells and macrophages.

In another aspect, the invention provides a method of treating a medical condition, the method comprising the step of administering to a subject in need thereof a therapeutically effective amount of the recombinant binding protein of the invention, the nucleic acid of the invention or the pharmaceutical composition of the invention.

In another aspect, the invention provides a method of treating a medical condition, the method comprising the step of administering to a subject in need thereof a therapeutically effective amount of the recombinant binding protein further comprising a binding agent with binding specificity for a disease-associated antigen, a nucleic acid encoding said recombinant binding protein or a pharmaceutical composition comprising said binding protein.

In one aspect, the invention relates to a pharmaceutical composition, a recombinant binding protein, or a nucleic acid according to the present invention for use in the treatment of a disease. For that purpose, the pharmaceutical composition, the nucleic acid or the recombinant binding protein according to the present invention is administered to a subject in need thereof, in a therapeutically effective amount. Administration may include topical administration, oral administration, or parenteral administration. The typical route of administration is parenteral administration. In parental administration, the pharmaceutical composition of this invention will be formulated in a unit dosage injectable form such as a solution, suspension, or emulsion, in association with the pharmaceutically acceptable excipients as defined above. The dosage and mode of administration will depend on the individual to be treated and the disease.

Further, any of the above-mentioned pharmaceutical composition, nucleic acid or recombinant protein is considered for use in the treatment of a disorder.

In one aspect, said recombinant binding protein or such other pharmaceutical composition described herein is applied intravenously. For parenteral application, the recombinant binding protein or said pharmaceutical composition can be injected as bolus injection or by slow infusion at a therapeutically effective amount.

In one aspect, the invention relates to the use of the recombinant binding protein of the invention, the nucleic acid of the invention or the pharmaceutical composition of the invention, as a medicament for the treatment of a disease. In one aspect, the invention relates to the use of the recombinant binding protein of the invention, the nucleic acid of the invention or the pharmaceutical composition of the invention for manufacturing of a medicament. In one aspect, the invention relates to the use of the recombinant binding protein of the invention, the nucleic acid of the invention or the pharmaceutical composition of the invention, for manufacturing of a medicament for the treatment of a disease. In one aspect, the invention relates to a process for the manufacturing of a medicament for the treatment of a disease, wherein the recombinant binding protein of the invention, the nucleic acid of the invention or the pharmaceutical composition of the invention is an active ingredient of the medicament. In one aspect, the invention relates to a method of treatment of a disease using the recombinant binding protein of the invention, the nucleic acid of the invention or the pharmaceutical composition of the invention.

In one aspect the invention further provides a use of such a recombinant binding protein for treating a medical condition of a subject in need thereof.

As used herein, said medical condition or disease is a cancer, preferably a liquid tumor, more preferably leukemia, even more preferably acute myeloid leukemia (AML). In a preferred embodiment, said cancer expresses one or more disease associated antigen (DAA), e.g. one or more tumor associated antigens (TAA) which is/are bound by the binding moieties comprised in the recombinant protein of the invention.

The recombinant binding protein of the present invention, nucleic acid of the invention or a pharmaceutical composition of the invention can also be used in combination with one or more other therapies known in the art. The term “use in combination with”, as used herein, shall refer to a co-administration, which is carried out under a given regimen. This includes synchronous administration of the different compounds as well as time-shifted administration of the different compounds (e.g., compound A is given once and compound B is given several times thereafter, or vice versa, or both compounds are given synchronously and one of the two is also given at later stages).

In one aspect, the invention relates to a kit comprising the recombinant binding protein of the invention. In one aspect, the invention relates to a kit comprising a nucleic acid encoding the recombinant binding protein of the invention. In one aspect, the invention relates to a kit comprising the pharmaceutical composition of the invention. In one aspect, the invention relates to a kit comprising the recombinant protein of the invention, and/or the nucleic acid of the invention, and/or the pharmaceutical composition of the invention. In one aspect, the invention relates to a kit comprising the recombinant protein comprising an ankyrin repeat domain with binding specificity for CD16a of the invention, for example SEQ ID NOs: 1 to 7, and/or a nucleic acid encoding the recombinant protein comprising an ankyrin repeat domain with binding specificity for CD16a, for example SEQ ID NOs: 1 to 7, and/or a pharmaceutical composition comprising the recombinant protein comprising an ankyrin repeat domain with binding specificity for CD16a, for example SEQ ID NOs: 1 to 7. In one aspect, the invention relates to a kit comprising the recombinant protein comprising any one of the amino acid sequences of SEQ ID NOs: 1 to 7, and/or a nucleic acid encoding said recombinant protein, and/or a pharmaceutical composition comprising the recombinant protein.

In one aspect, the invention relates to a method for producing a recombinant binding protein of the present invention. In one aspect, the invention relates to a method for producing a recombinant binding protein, for example a recombinant binding protein comprising the amino acid sequence of any one of SEQ ID NOs: 1 to 23, the method comprising the steps of (i) expressing said recombinant binding protein in a suitable host cell (e.g., bacteria), and (ii) purifying said recombinant binding protein (e.g., using chromatography). Said method may comprise additional steps. An example of such a method of producing a recombinant binding protein of the present invention is described in Example 1.

The invention is not restricted to the particular aspects described in the Examples.

### Definitions

Unless defined otherwise herein, all technical and scientific terms used herein shall have the meanings that are commonly understood by those of ordinary skill in the art to which the present invention belongs.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g., by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g., produced by gene synthesis) encoding a polypeptide can be cloned into a bacterial expression plasmid (e.g., pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g., Escherichia coli), these bacteria can produce the polypeptide(s) encoded by this recombinant DNA. The correspondingly produced polypeptide or protein is called a recombinant polypeptide or recombinant protein.

In the context of the present invention, the term “binding protein” refers to a protein comprising a binding domain. A binding protein may also comprise two, three, four, five or more binding domains. Preferably, said binding protein is a recombinant binding protein. Binding proteins of the instant invention comprise an ankyrin repeat domain with binding specificity for CD16a.

Furthermore, any such binding protein may comprise additional polypeptides (such as e.g., polypeptide tags, peptide linkers, fusion to other proteinaceous domains with binding specificity, cytokines, hormones, or antagonists), or chemical modifications (such as coupling to polyethylene-glycol, toxins (e.g., DM1 from Immunogen), small molecules, antibiotics and alike) well known to the person skilled in the art.

The term “binding domain” means a protein domain exhibiting binding specificity for a target. Preferably, said binding domain is a recombinant binding domain.

As used herein, the term “target” refers to an individual molecule such as a nucleic acid molecule, a polypeptide or protein, a carbohydrate, or any other naturally occurring molecule, including any part of such individual molecule, or to complexes of two or more of such molecules, or to a whole cell or a tissue sample, or to any non-natural compound. Preferably, a target is a naturally occurring or non-natural polypeptide or protein, or a polypeptide or protein containing chemical modifications, for example, naturally occurring or non-natural phosphorylation, acetylation, or methylation. In the context of the present invention, CD16a protein expressed on NK cells is target of CD16a-specific binding proteins. Preferably, the target is human CD16a.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S-S bridges of cysteines. Polypeptides are well-known to the person skilled in the art.

Patent application WO2002/020565 and Forrer et al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains.

The term “repeat domain” refers to a protein domain comprising two or more consecutive repeat modules as structural units, wherein said repeat modules have structural and sequence homology. Preferably, a repeat domain further comprises an N-terminal and/or a C-terminal capping module. For clarity, a capping module can be a repeat module. Such repeat domains, repeat modules, and capping modules, sequence motives, as well as structural homology and sequence homology are well known to the practitioner in the art from examples of ankyrin repeat domains (WO2002/020565), leucine-rich repeat domains (WO2002/020565), tetratricopeptide repeat domains (Main, E. R., Xiong, Y., Cocco, M. J., D'Andrea, L., Regan, L., Structure 11(5), 497-508, 2003), and armadillo repeat domains (WO2009/040338). It is further well known to the practitioner in the art, that such repeat domains are different from proteins comprising repeated amino acid sequences, where every repeated amino acid sequence is able to form an individual domain (for example FN3 domains of Fibronectin).

The term “ankyrin repeat domain” refers to a repeat domain comprising two or more consecutive ankyrin repeat modules as structural units. Ankyrin repeat domains may be modularly assembled into larger ankyrin repeat proteins, optionally with half-life extension domains, using standard recombinant DNA technologies (see, e.g., Forrer, P., et al., FEBS letters 539, 2-6, 2003, WO2002/020565, WO2016/156596, WO2018/054971). The term “construct” as used herein refers to a recombinant binding protein comprising one or more designed ankyrin repeat domain and optionally a peptide linker and/or tag sequence. Peptide linkers and tag sequences are known to the person skilled in the art.

The term “designed” as used in designed repeat protein, designed repeat domain and the like refers to the property that such repeat proteins and repeat domains, respectively, are man-made and do not occur in nature. The binding proteins of the instant invention are designed repeat proteins and they comprise at least one designed ankyrin repeat domain. Preferably, the designed repeat domain is a designed ankyrin repeat domain.

The term “target interaction residues” refers to amino acid residues of a repeat module, which contribute to the direct interaction with a target.

The term “framework residues” refers to amino acid residues of a repeat module, which contribute to the folding topology, i.e. which contribute to the fold of said repeat module or which contribute to the interaction with a neighbouring module. Such contribution may be the interaction with other residues in the repeat module, or the influence on the polypeptide backbone conformation as found in α-helices or β-sheets, or the participation in amino acid stretches forming linear polypeptides or loops. Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “repeat modules” refers to the repeated amino acid sequence and structural units of the designed repeat domains, which are originally derived from the repeat units of naturally occurring repeat proteins. Each repeat module comprised in a repeat domain is derived from one or more repeat units of a family or subfamily of naturally occurring repeat proteins, e.g. the family of ankyrin repeat proteins. Furthermore, each repeat module comprised in a repeat domain may comprise a “repeat sequence motif” deduced from homologous repeat modules obtained from repeat domains selected on a target, e.g. as described in Example 1, and having the same target specificity.

Accordingly, the term “ankyrin repeat module” refers to a repeat module, which is originally derived from the repeat units of naturally occurring ankyrin repeat proteins. Ankyrin repeat proteins are well known to the person skilled in the art.

Repeat modules may comprise positions with amino acid residues which have not been randomized in a library for the purpose of selecting target-specific repeat domains (“non-randomized positions”) and positions with amino acid residues which have been randomized in the library for the purpose of selecting target-specific repeat domains (“randomized positions”). The non-randomized positions comprise framework residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline. For the purpose of this patent application, amino acid residues 3, 4, 6, 11, 14 and 15 of SEQ ID NOs: 27 to 41, 52 to 57 and 97, amino acid residues 4, 8, 11 and 12 of SEQ ID NOs: 42 to 46 and 58 to 61, and amino acid residues 3, 4, 6, 14 and 15 of SEQ ID NOs: 47 to 51 and 62 to 68 are randomized positions of the ankyrin repeat modules of the instant invention. Randomized positions are generally not substituted by other amino acids in the variants of the module sequences disclosed herein.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

The terms “binding specificity”, “has binding specificity for a target”, “specifically binding to a target”, “binding to a target with high specificity”, “specific for a target”, “target specificity”, or “specifically binds” and the like means that a binding protein or binding domain binds to a target with a lower dissociation constant (i.e. it binds with higher affinity) than it binds to an unrelated protein such as the E. coli maltose binding protein (MBP). Preferably, the dissociation constant (“K_{D}”) for the target is at least 10²; more preferably, at least 10³; more preferably, at least 10⁴; or more preferably, at least 10⁵ times lower than the corresponding dissociation constant for MBP. Methods to determine dissociation constants of protein-protein interactions, such as surface plasmon resonance (SPR) based technologies (e.g., SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS.

Binding of any molecule to another is governed by two forces, namely the association rate (kₒₙ) and the dissociation rate (k_{off}). The affinity of any binder [B] to a target [T] can then be expressed by the equilibrium dissociation constant K_{D}, which is the quotient of k_{off}/kₒₙ.

$$[ B ] + [ T ] {{⇌}^{{k}_{on}}}_{{k}_{off}} [ BT ]$$

kₒₙ is a second-order rate constant of the binding reaction, with the unit M⁻¹ s⁻¹, whereas the dissociation reaction k_{off} is a first-order rate constant with the unit s⁻¹. From this it becomes clear that the association reaction depends on the concentration of the reactants, whereas the dissociation is independent of the concentration, following a simple exponential decay function.

A variety of methods of measuring binding affinity are known in the art, any of which can be used for purposes of the present invention. For example, as exemplified herein, the binding affinity of a particular binding moiety to a drug molecule target can be expressed as K_{D} value, which refers to the dissociation constant of the binding moiety and the drug molecule target. K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M), and the smaller the K_{D}, the stronger the affinity of binding.

K_{D} values can be determined using any suitable method. One exemplary method for measuring K_{D} is surface plasmon resonance (SPR) (see, e.g., Nguyen et al. Sensors (Basel). 2015 May 5; 15(5):10481-510). K_{D} value may be measured by SPR using a biosensor system such as a BIACORE® system. BIAcore kinetic analysis comprises, e.g., analysing the binding and dissociation of an antigen from chips with immobilized molecules (e.g., molecules comprising epitope binding domains), on their surface. Another method for determining the K_{D} of a protein is by using Bio-Layer Interferometry (see, e.g., Shah et al. J Vis Exp. 2014; (84): 51383). A K_{D} value may be measured using OCTET® technology (Octet QKe system, ForteBio). Alternatively, or in addition, a KinExA® (Kinetic Exclusion Assay) assay, available from Sapidyne Instruments (Boise, Id.) can also be used. Any method suitable for assessing the binding affinity between two binding partners is encompassed herein.

A typical and preferred determination of dissociation constants (K_{D}) of the inventive recombinant binding proteins with binding specificity for CD16a by Surface Plasmon Resonance (SPR) analysis is described in Example 6.

The term “binding agent” or “binding moiety” refers to any molecule capable of specifically binding a target molecule. Binding agents include, for example, antibodies, antibody fragments, aptamers, peptides (e.g., Williams et al., J Biol Chem 266:5182-5190 (1991)), alternative scaffolds, antibody mimics, repeat proteins, e.g., designed ankyrin repeat proteins, receptor proteins and any other naturally occurring interaction partners of the target molecule, and can comprise natural proteins and proteins modified or genetically engineered, e.g., to include non-natural residues and/or to lack natural residues.

The term “PBS” means a phosphate buffered water solution containing 137 mM NaCl, 10 mM phosphate and 2.7 mM KCl and having a pH of 7.4.

The term “about” as used herein is equivalent to ±10% of a given numerical value, unless otherwise stated.

Preferably, clearance, and/or exposure, and/or terminal half-life are assessed in a mammal, more preferably mouse and/or cynomolgus monkey, more preferably cynomolgus monkey. Clearance, and/or exposure, and/or terminal half-life may be assessed as described in Example 7. Preferably, when measuring the clearance, and/or exposure, and/or terminal half-life in mouse, the evaluation is done considering the data up to 48 h post-injection. More preferably, the evaluation of terminal half-life in mouse is calculated from 24 h to 48 h. Preferably, when measuring the clearance, and/or exposure, and/or terminal half-life in cynomolgus monkey, the evaluation is done considering the data up to day 7 post-injection. More preferably, the evaluation of terminal half-life in cynomolgus monkey is calculated from day 1 to day 5. The person skilled in the art further is able to identify effects such as target-mediated clearance and consider them when calculating the terminal half-life. The term “terminal half-life” of a drug such as a recombinant binding protein of the invention refers to the time required to reach half the plasma concentration of the drug applied to a mammal after reaching pseudo-equilibrium (for example calculated from 24 hours to 48 hours in mouse or calculated from day 1 to day 5 in cynomolgus monkey). Terminal half-life is not defined as the time required to eliminate half the dose of the drug administered to the mammal. The term terminal half-life is well known to the person skilled in the art. Preferably, pharmacokinetic comparison is done at any dose, more preferably at equivalent dose (i.e., same mg/kg dose) or equimolar dose (i.e., same mol/kg dose), more preferably at equimolar dose (i.e., same mol/kg dose). It is understood by the person skilled in the art that equivalent and/or equimolar dosing in animals is subject to experimental dose variations of at least about 20%, more preferably about 30%, about 40%, about 50%, about 60%, about 70%, about 80%, about 90%, or about 100%. Preferably, a dose used for pharmacokinetic measurement is selected from about 0.001 to about 1000 mg/kg, more preferably about 0.01 to about 100 mg/kg, more preferably about 0.1 to about 50 mg/kg, more preferably about 0.5 to about 10 mg/kg.

The term “CD16a” refers to the Fc gamma III-A receptor. This is a receptor for IgGs. The amino acid sequence of human CD16a (hCD16a) is shown in UniProt (www.uniprot.org) Ref. No. P08637.

The term “CD16b” refers to the Fc gamma III-B receptor. CD16b is the only Fc receptor anchored to the cell membrane by a glycosyl-phosphatidylinositol (GPI) linker and upon neutrophil activation is found soluble in serum. The amino acid sequence of human CD16b (hCD16b) is shown in UniProt (www.uniprot.org) Ref. No. 075015.

The term “tumor-localized activation of immune cells” means that innate immune cells are activated preferentially in tumor tissue as compared to a non-tumor tissue.

Furthermore, the term “peptide” also encompasses peptides modified by, e.g., glycosylation, and proteins comprising two or more polypeptide chains, each of length of 4 to 600 amino acids long, cross-linked by, e.g., disulfide bonds, such as, e.g., insulin and immunoglobulins. The term “chemical or biochemical agent” is intended to include any naturally occurring or synthetic compound that may be administered to a recipient.

The term “medical condition” (or disorder or disease) includes autoimmune disorders, inflammatory disorders, retinopathies (particularly proliferative retinopathies), neurodegenerative disorders, infections, metabolic diseases, and neoplastic diseases. Any of the recombinant binding proteins described herein may be used for the preparation of a medicament for the treatment of such a disorder, particularly a disorder selected from the group consisting of an autoimmune disorder, an inflammatory disorder, an immune disorder, and a neoplastic disease. A “medical condition” may be one that is characterized by inappropriate cell proliferation. A medical condition may be a hyperproliferative condition. The invention particularly relates to a method of treating a medical condition, the method comprising the step of administering, to a patient in need of such treatment, a therapeutically effective amount of a recombinant binding protein or said pharmaceutical composition of the invention. In a preferred embodiment said medical condition is a neoplastic disease. The term “neoplastic disease”, as used herein, refers to an abnormal state or condition of cells or tissue characterized by rapidly proliferating cell growth or neoplasm. In one embodiment said medical condition is a malignant neoplastic disease. In one embodiment said medical condition is a cancer, preferably leukaemia, more preferably acute myeloid leukaemia. The term “therapeutically effective amount” means an amount that is sufficient to produce a desired effect on a patient.

The term “antibody” means not only intact antibody molecules, but also any fragments and variants of antibody molecules that retain immunogen-binding ability. Such fragments and variants are also well known in the art and are regularly employed both in vitro and in vivo. Accordingly, the term “antibody” encompasses intact immunoglobulin molecules, antibody fragments such as, e.g., Fab, Fab′, F(ab′)₂, and single chain V region fragments (scFv), bispecific antibodies, chimeric antibodies, antibody fusion polypeptides, and unconventional antibodies.

The terms “cancer” and “cancerous” are used herein to refer to or describe the physiological condition in mammals that is typically characterized by unregulated cell growth. Cancer encompasses solid tumors and liquid tumors, as well as primary tumors and metastases. A “tumor” comprises one or more cancerous cells. Solid tumors typically also comprise tumor stroma. Examples of cancer include, but are not limited to, primary and metastatic carcinoma, lymphoma, blastoma, sarcoma, and leukemia, and any other epithelial and lymphoid malignancies. More particular examples of such cancers include brain cancer, bladder cancer, breast cancer, ovarian cancer, clear cell kidney cancer, head/neck squamous cell carcinoma, lung adenocarcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, colorectal cancer, or gastric cancer.

Throughout this specification and the claims which follow, and unless the context requires otherwise, the terms “comprising”, “having” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

### EXAMPLES

Starting materials and reagents disclosed below are known to those skilled in the art, are commercially available and/or can be prepared using well-known techniques.

### Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. coli XL1-blue (Stratagene, USA) or BL21 (Novagen, USA). Cell lines used in Example 3: Human JIMT-1 (DSMZ: ACC-589), Human HT29 (ATCC HTB-38), Human SKOV3 (ATCC HTB-77), Human MDA-MB-175 (ATCC HTB-25), Human MDA-MB-231 (ATCC HTB-26) and Human MDA-MB-468 (ATCC HTB-132).

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

### Designed Ankyrin Repeat Protein Libraries

Methods to generate designed ankyrin repeat protein libraries have been described, e.g., in U.S. Pat. No. 7,417,130; Binz et al., J. Mol. Biol. 332, 489-503, 2003; Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module and a fixed C-terminal capping module or a randomized C-terminal capping module. Preferably, such libraries are assembled to not have any of the amino acids C, G, M, N (in front of a G residue) and P at randomized positions of repeat or capping modules.

Furthermore, such randomized modules in such libraries may comprise additional polypeptide loop insertions with randomized amino acid positions. Examples of such polypeptide loop insertions are complementarity determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the boarder of two ankyrin repeats, ankyrin repeat proteins libraries may contain randomized loops (with fixed and randomized positions) of variable length (e.g., 1 to 20 amino acids) inserted in one or more beta-turns of an ankyrin repeat domain. Any such N-terminal capping module of an ankyrin repeat protein library preferably possesses the RILLAA, RILLKA or RELLKA motif (e.g., present from position 19 to 24 in SEQ ID NO: 42) and any such C-terminal capping module of an ankyrin repeat protein library preferably possesses the KLN, KLA or KAA motif (e.g., present at the last three amino acids in SEQ ID NO: 47). SEQ ID NO: 42 to 46 provide examples of N-terminal capping modules comprising the RILLAA, RILLKA or RELLKA motif, and SEQ ID NO: 47 to 51 provide examples of C-terminal capping modules comprising the KLN, KLA or KAA motif.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V2O, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

### Example 1: Selection of Binding Proteins Comprising an Ankyrin Repeat Domain with Binding Specificity for CD16a

Using ribosome display (Hanes, J. and Plückthun, A., PNAS 94, 4937-42, 1997), many ankyrin repeat proteins with binding specificity for human CD16a (hCD16a) were selected from DARPin libraries similar as described by Binz et al. 2004 (loc. cit.). The binding of the selected clones toward recombinant CD16a target was assessed by crude extract Homogeneous Time Resolved Fluorescence (HTRF), indicating that hundreds of hCD16a-specific binding proteins were successfully selected. For example, the ankyrin repeat domains of SEQ ID NOs: 1-6 constitute amino acid sequences of selected binding proteins comprising an ankyrin repeat domain with binding specificity for CD16a. SEQ ID NOs: 27 to 51 constitute ankyrin repeat modules of selected binding proteins with binding specificity for hCD16a.

### Selection of CD16a-Specific Ankyrin Repeat Proteins by Ribosome Display

The selection of CD16a-specific ankyrin repeat proteins was performed by ribosome display (Hanes and Plückthun, loc. cit.) using the biotinylated extracellular domains of human CD16a (UniProt entry P08637) as target protein, libraries of ankyrin repeat proteins as described above, and established protocols (See, e.g., Zahnd, C., Amstutz, P. and Plückthun, A., Nat. Methods 4, 69-79, 2007). A variant of the CD16a sequence having a Val at position 176 (when counting the signal sequence) was used for selection. This corresponds to Val in position 160 of SEQ ID NO: 72 and is also herein referred to as CD16aV. A variant of the CD16a sequence having a Phe at this same position was used in the screening steps and is referred to as CD16aF (SEQ ID NO: 75). These CD16a targets (AcroBiosystems) include a C-terminal His-tag and were biotinylated using the Sulfo-NHS-LC-Biotin kit (Pierce). In total four to six rounds of standard ribosome selections were employed, using decreasing target concentration and increasing washing stringency to increase selection pressure from round 1 to round 6 (Binz et al. 2004, loc. cit.). A deselection strategy (or negative selection) was applied in each round by using a mix of two CD16b allotypes referred to as CD16b_NA1 and CD16b_NA2 (SEQ ID NOs: 73 and 74 respectively).

To enrich high affinity CD16a-specific ankyrin repeat proteins, the output from the sixth round of standard ribosome display selection (above) was subjected to an off-rate selection round using non-biotinylated target with increased selection stringency (Zahnd, 2007, loc. cit.). A final standard selection round was performed after the off-rate selection round to amplify and recover the off-rate selected binding proteins.

### Selected Clones Bind Specifically to Human CD16a as Shown by Crude Extract HTRF

Individual selected ankyrin repeat proteins specifically binding to CD16aV and/or CD16aF in solution were identified by Homogeneous Time Resolved Fluorescence (HTRF) assay using crude extracts of ankyrin repeat protein-expressing Escherichia coli cells using standard protocols. Ankyrin repeat protein clones selected by ribosome display were cloned into derivatives of the pQE30 (Qiagen) expression vector containing a N-terminal Flag tag (SEQ ID NO: 71), transformed into E. coli XL1-Blue (Stratagene), plated on LB-agar (containing 1% glucose and 50 μg/ml ampicillin) and then incubated overnight at 37° C. Single colonies were picked into a 96 well plate (each clone in a single well) containing 160 μl growth medium (TB containing 1% glucose and 50 μg/ml ampicillin) and incubated overnight at 37° C., shaking at 800 rpm. 150 μl of fresh TB medium containing 50 μg/ml ampicillin was inoculated with 8.5 μl of the overnight culture in a fresh 96-well plate. After incubation for 120 minutes at 37° C. and 700 rpm, expression was induced with IPTG (0.5 mM final concentration) and continued for 4 hours. Cells were harvested and the pellets were frozen at −20° C. overnight before resuspension in 8 μl B-PERII (Thermo Scientific) and incubation for 1 hour at room temperature with shaking (900 rpm). Then, 160 μl PBS was added and cell debris was removed by centrifugation (3220 g for 15 min).

In parallel, further HTRF assays were similarly performed with ankyrin repeat protein clones cloned into derivatives of the pQE30 (Qiagen) expression vector containing a N-terminal HER2 specific ankyrin repeat domain and Flag tag. The clones in this screening branch were also tested for cross-reactivity to Cynomolgus CD16 (cCD16) (UniProt ID Nr: A3RFZ7). The extracellular domain of the cCD16 has 92% sequence identity to human CD16, and was biotinylated and C-terminally His- and Avi-tagged, corresponding to SEQ ID NO: 76 (AcroBiosystems).

CD16aV, CD16aF and cCD16 (all biotinylated) were used for the assessment of desired target binding and CD16b_NA1 and CD16b_NA2 (both biotinylated) as control to exclude CD16b binders. Representative conditions are as follows: the extract of each lysed clone was applied as a 1:1000 dilution (final concentration) in PBSTB (PBS supplemented with 0.1% Tween 20® and 0.2% (w/v) BSA, pH 7.4) together with 4 nM (final concentration) biotinylated target (i.e CD16aV, CD16aF, cCD16, CD16b_NA1 or CD16b_NA2), 1:400 (final concentration) of anti-strep-Tb HTRF antibody—FRET donor conjugate (Cisbio) and 1:400 (final concentration) of anti-Flag-D2 antibody FRET acceptor conjugate (Cisbio) to a well of 384 well plate and incubated for 60 minutes at RT. The HTRF was read-out on a Tecan M1000 using a 340 nm excitation wavelength and a 665±10 nm emission filter. Screening of several hundred clones by such a crude cell extract HTRF revealed ankyrin repeat domains with specificity for hCD16a. Amino acid sequences of selected ankyrin repeat domains that specifically bind to hCD16a are provided in SEQ ID NOs: 1-6:
DARPin #1 (SEQ ID NO: 1);
DARPin #2 (SEQ ID NO: 2);
DARPin #3 (SEQ ID NO: 3);
DARPin #4 (SEQ ID NO: 4);
DARPin #5 (SEQ ID NO: 5); and
DARPin #6 (SEQ ID NO: 6).

These DARPin optionally comprise additionally a G, an S, or a GS sequence at their N-terminus.

### Expression of CD16a-Specific Ankyrin Repeat Proteins

For further analysis, the selected clones showing specific CD16a binding in the crude cell extract HTRF, as described above, were expressed in E. coli cells, with a His-tag (SEQ ID NO: 69) fused to their N-terminus for easy purification. Expressed proteins were purified according to standard protocols. 5 ml of stationary overnight cultures (TB, 1% glucose, 50 mg/l of ampicillin; 37° C.) were used to inoculate 150 ml cultures (TB, 50 mg/l ampicillin, 37° C.). At an absorbance of 1.0 to 1.5 at 600 nm, the cultures were induced with 1 mM IPTG and incubated at 37° C. for 4-5 h while shaking. The cultures were centrifuged, and the resulting pellets were re-suspended in 10 ml of TBS500 (50 mM Tris-HCl, 500 mM NaCl, pH 8) and stored at −20° C., before they were thawed, mixed with 50 KU DNase/ml and 1 mg/mL of lysozyme and lysed (sonication or French press). Following the lysis, the samples were centrifuged and the supernatant was collected and filtrated. Imidazole (20 mM final concentration) was added to the homogenate. Proteins were purified over a bench-top Ni-nitrilotriacetic (Ni-NTA) acid column followed by a desalting column (NAP-25) step according to standard protocols and resins known to the person skilled in the art. Highly soluble ankyrin repeat proteins were purified from E. coli culture (up to 200 mg ankyrin repeat protein per litre of culture) with a purity >95% as estimated from 4-12% SDS PAGE. These mono-domain DARPins were characterized biophysically by size exclusion chromatography, ProteOn surface plasmon resonance (SPR) target affinity assessment, ELISA, target protein-competition HTRF experiments, and/or SDS-PAGE.

Generation of Affinity Matured CD16 Specific Binding Proteins with Binding Specificity for CD16a.

In further development of the initially identified CD16a specific binding proteins, variants with increased affinity to and/or reduced off-rate from target protein were generated using affinity maturation. Thereby, one initially identified binding protein DARPin #2 (the “parental” binding protein) was selected as a suitable starting point for affinity maturation. The affinity maturation procedure entailed saturation mutagenesis of each randomized position and one non-randomized position (i.e position 5 in 2^{nd} internal repeat) of the ankyrin repeat domain used as a starting point. Sequences generated by the affinity maturation procedure were screened for lower off-rates by competition HTRF. In short: Single amino acid point mutations variants were generated by standard QuikChange PCR on parental plasmid using primers with a single NNK degenerated codon to introduce all 20 amino acids at a potential binding position. Crude extracts of ankyrin repeat proteins, containing an N-terminal Flag-tag (SEQ ID NO: 71) were incubated with the biotinylated target before addition of excess of non-flag-tagged parental CD16a-specific binding proteins and measurement of HTRF signal over time. Beneficial mutations identified based on higher HTRF signals compared to parental protein, were combined in the binding proteins by protein engineering. This way affinity matured DARPin #7 was generated, originating from DARPin #2.

### Example 2: Cell Binding Specificity Profile of Selected CD16a-Specific DARPins

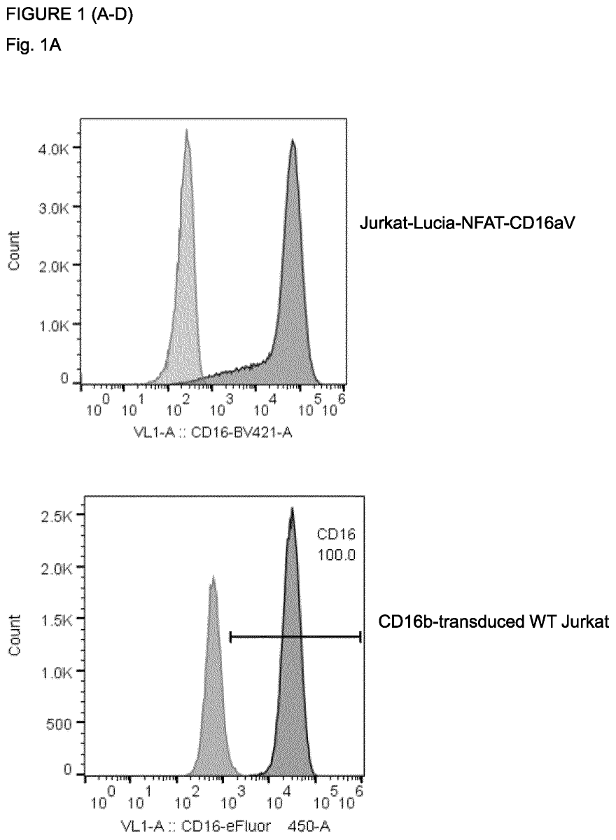

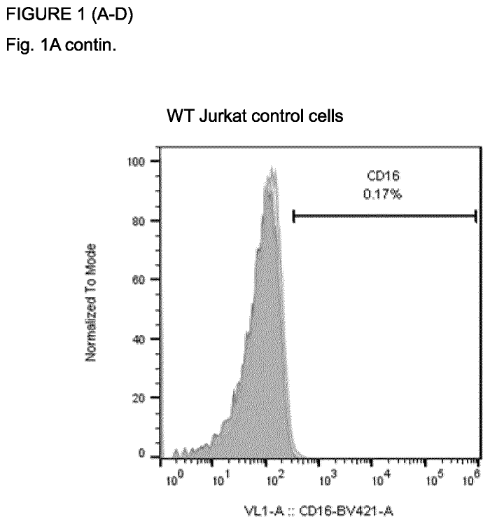

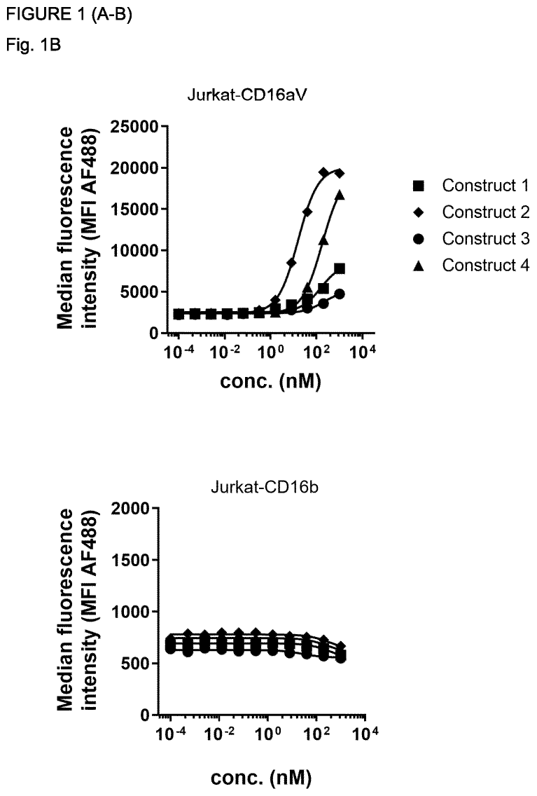

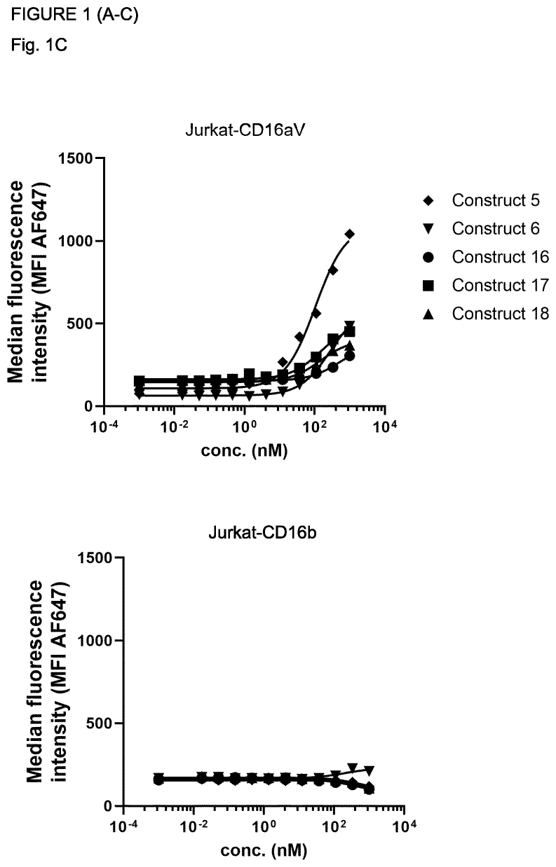

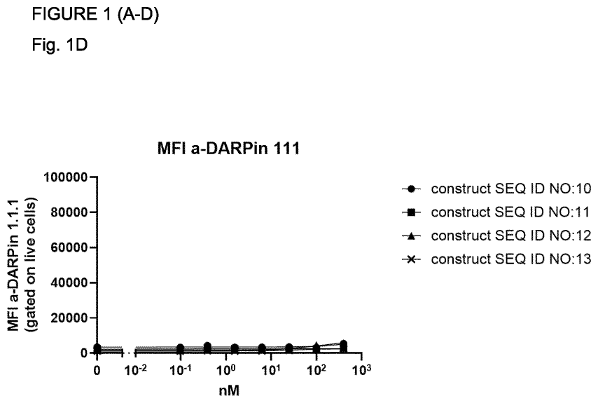

Binding specificity of selected CD16a binders as generated in Example 1 was assessed in reporter cell binding experiments. Jurkat-Lucia-NFAT-CD16 Reporter cells (InvivoGen) express the CD16aV allotype. Depending on the sources, the mutations in the CD16a allotypes can also be referred to as the V158 (see e.g. Bruhns P. et al., 2009. Blood. 113(16):3716; Nagelkerke S. Q. et al., 2019. Front. Immunol. 10:2237). In addition, cell lines stably expressing CD16b were generated by lentiviral transduction. Jurkat WT cell line from ATCC was used and lentiviruses encoding full length human CD16b were purchased at G&P Biosciences (USA). WT Jurkat cells were transduced, sorted by FACS and expanded according to standard protocols. Single clones with a clear expression of CD16b were obtained. Expression of CD16aV and CD16b from the respective cell lines was measured by flow cytometry and is shown in FIG. 1A. Briefly, transduced cells were washed with PBS, centrifuged at 300 g for 5 min and first stained for viability with the live/dead fixable green dead cell stain Kit-AF488 (Thermo). After 20 min incubation at 4° C., cells were washed with FACS buffer and centrifuged at 300 g for 5 min followed by the stain with an anti-CD16 antibody which binds to both CD16 forms, CD16a and CD16b (BV421 mouse anti-human CD16 clone 3G8, mouse IgG1, BD Biosciences). A mouse IgG-1-BV421 was used as isotype control. Gating was done on live/dead cells and CD16a/CD16b positive cells. FACS data were analyzed using FlowJo software.

### 2.1 First Experiment

CD16a-specific binders were formatted as mono domain (1D) or bi-specific binders (two domains or 2D). The 2D format is selected as a model NK-cell engager molecule, wherein the second binding domain is an ankyrin repeat domain having binding specificity for HER2. Representative tested constructs which were cloned and expressed according to standard protocols are shown in Table 1. In this example, Construct No. 3 comprises the HER2 binding domain C-terminally of the CD16a binding domain and in Construct No. 4 the HER2 binding domain is located N-terminally of the CD16a binding domain. All constructs in Table 1 further comprise a N-terminal His Tag of SEQ ID NO: 69.

TABLE 1

| Construct No. | Format | CD16a binder | HER2 binder | Linker |
|---|---|---|---|---|
| 1 | 1D (CD16a) | SEQ ID NO: 2 | NA | NA |
| | | (DARPin #2) | | |
| 2 | 1D (CD16a) | SEQ ID NO: 3 | NA | NA |
| | | (DARPin #3) | | |
| 3 (SEQ ID NO: 13) | 2D (CD16a × HER2) | SEQ ID NO: 2 | SEQ ID NO: 9 | PT linker (SEQ |
| | | (DARPin #2) | | ID NO: 70) |
| 4 (SEQ ID NO: 15) | 2D (HER2 × CD16a) | SEQ ID NO: 3 | SEQ ID NO: 9 | PT linker (SEQ |
| | | (DARPin #3) | | ID NO: 70) |

Serial dilutions (5-fold) of the constructs were incubated 30 min at 4° C. with 50000 cells in a 96-well plate. Detection antibody (Penta-His Alexa Fluor 488 Conjugate, Qiagen) targeting the penta-His tag of the constructs was subsequently added to the wells (2 μg/ml diluted in FACS buffer) and incubated at 4° C. for 30 min. Cold buffer was added, then cells were spun down prior to supernatant removal. Cells were then fixed with Cell fix buffer (BD) and signal was measured by flow cytometry. Resulting MFI values are shown with a standard non-linear regression three parameter logistic model fit in FIG. 1B.

As can be seen in FIG. 1B, dose-dependent binding is observed for the tested constructs both in 1D and 2D formats to CD16a expressing cells and no binding is observed to CD16b expressing cells, supporting the target specificity of the binders of the invention.

### 2.2 Second Experiment

An additional round of similar tests was performed with further 2D constructs shown in Table 2a. Constructs Nos. 5 and 6 further comprise a N-terminal His Tag of SEQ ID NO: 69 and Constructs Nos. 16, 17 and 18 further comprise a N-terminal Flag Tag of SEQ ID NO: 71. Apart from the dilutions steps and detection antibody dye which was AF647 in this experiment (Mouse IgG1 Penta-His Alexa Fluor 647 Conjugate, Qiagen), the conditions were the same as described in section 2.1. Similar observations as in section 2.1 were made, supporting target specificity of the binders of the invention (FIG. 1C).

TABLE 2a

| Construct No. | Format | CD16a binder | HER2 binder | Linker |
|---|---|---|---|---|
| 5 (SEQ ID NO: 11) | 2D (HER2 × CD16a) | SEQ ID NO: 2 | SEQ ID NO: 9 | PT linker (SEQ |
| | | (DARPin #2) | | ID NO: 70) |
| 6 (SEQ ID NO: 17) | 2D (CD16a × HER2) | SEQ ID NO: 3 | SEQ ID NO: 9 | PT linker (SEQ |
| | | (DARPin #3) | | ID NO: 70) |
| 16 (SEQ ID NO: 19) | 2D (HER2 × CD16a) | SEQ ID NO: 4 | SEQ ID NO: 9 | PT linker (SEQ |
| | | (DARPin #4) | | ID NO: 70) |
| 17 (SEQ ID NO: 20) | 2D (HER2 × CD16a) | SEQ ID NO: 5 | SEQ ID NO: 9 | PT linker (SEQ |
| | | (DARPin #5) | | ID NO: 70) |
| 18 (SEQ ID NO: 21) | 2D (HER2 × CD16a) | SEQ ID NO: 6 | SEQ ID NO: 9 | PT linker (SEQ |
| | | (DARPin #6) | | ID NO: 70) |

### 2.3. Third Experiment

Cell binding of four selected NK cell engagers with binding specificity for CD16a and HER2 in a 2D format, as shown on Table 2b, was tested on Jurkat-NFAT-CD32a expressing CD32a at their surface (Invivogen jktl-nfat-cd32). Serial dilutions (4-fold) of the constructs were incubated 30 min at 4° C. with 50000 cells in a 96-well plate. Detection antibody (anti-DARPin 1.1.1 Alexa Fluor 467) was subsequently added to the wells (2 μg/ml diluted in FACS buffer) and incubated at 4° C. for 30 min. Cold buffer was added, then cells were spun down prior to supernatant removal. Cells were then fixed with Cell fix buffer (BD) and signal was measured by flow cytometry. Resulting MFI (Median Fluorescent Intensity) values are shown with a standard non-linear regression three parameter logistic model fit in FIG. 1D. These data support target specificity of the binders.

TABLE 2b

| Construct | | | HER2 | |
|---|---|---|---|---|
| No. | Format | CD16a binder | binder | Linker |
| 7 (SEQ ID | 2D (HER2 × | SEQ ID NO: 2 | SEQ ID | PT linker (SEQ |
| NO: 10) | CD16a) | (DARPin #2) | NO: 8 | ID NO: 70) |
| 5 (SEQ ID | 2D (HER2 × | SEQ ID NO: 2 | SEQ ID | PT linker (SEQ |
| NO: 11) | CD16a) | (DARPin #2) | NO: 9 | ID NO: 70) |
| 8 (SEQ ID | 2D (CD16a × | SEQ ID NO: 2 | SEQ ID | PT linker (SEQ |
| NO: 12) | HER2) | (DARPin #2) | NO: 8 | ID NO: 70) |
| 3 (SEQ ID | 2D (CD16a × | SEQ ID NO: 2 | SEQ ID | PT linker (SEQ |
| NO: 13) | HER2) | (DARPin #2) | NO: 9 | ID NO: 70) |

### Example 3: Functional Testing of CD16a-Specific DARPins in ADCC and ADCP Reporter Cell Assays

### A. Functional Testing of CD16a-Specific DARPins in ADCC Reporter Cell Assay

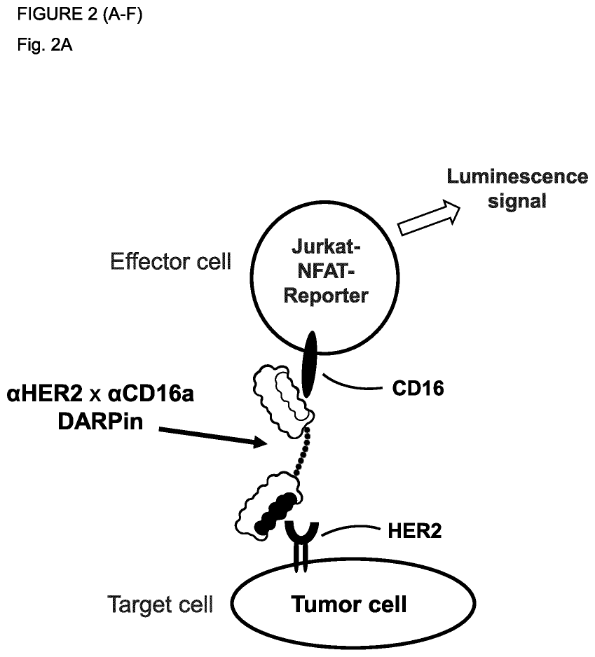

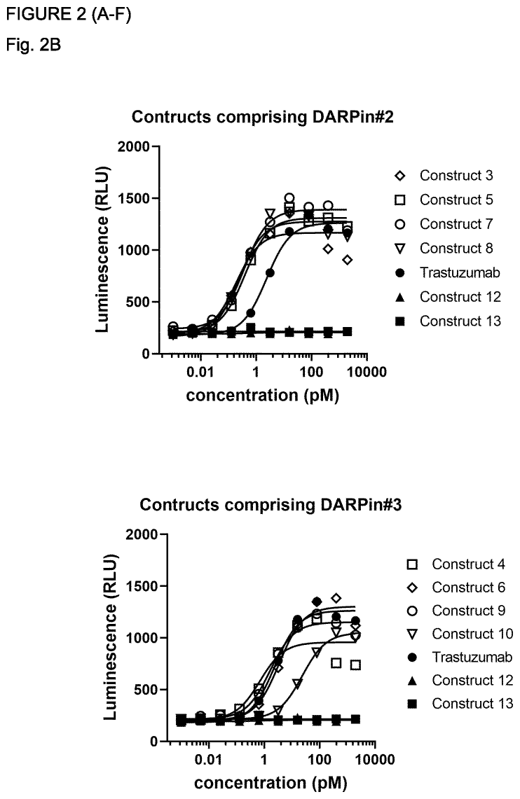

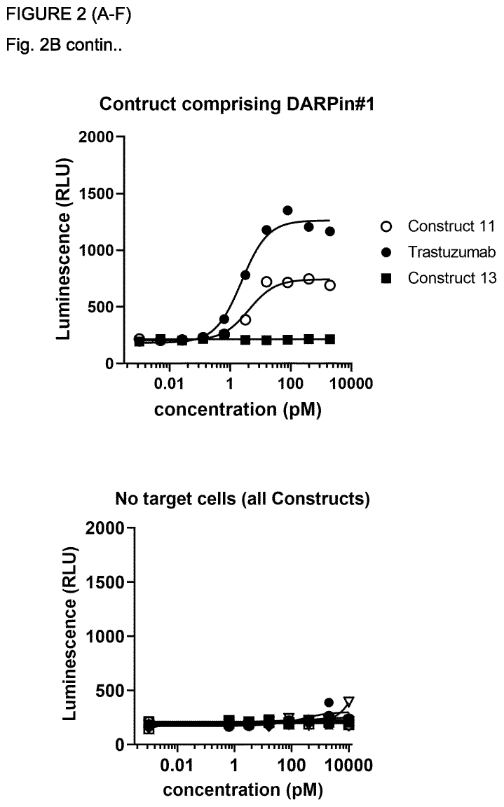

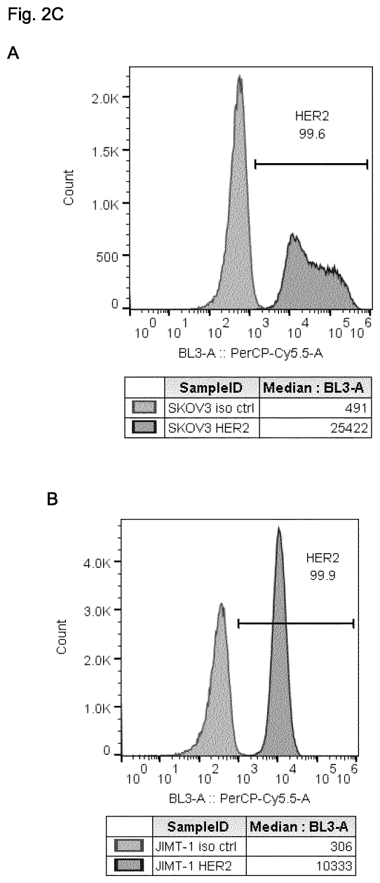

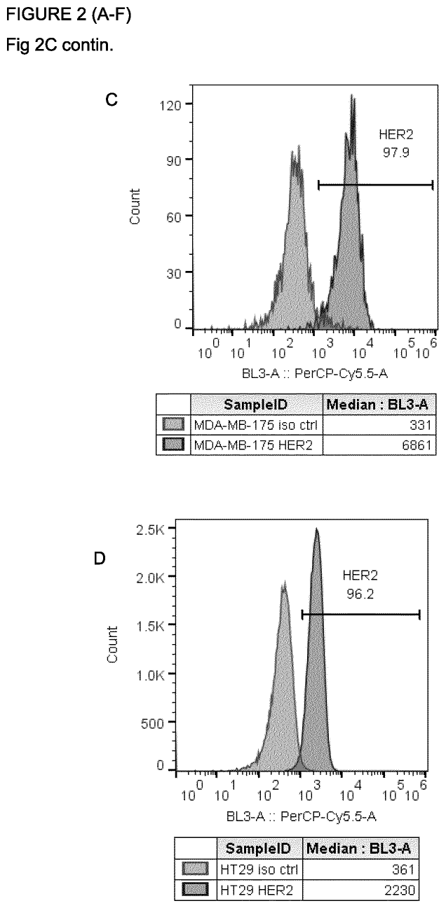

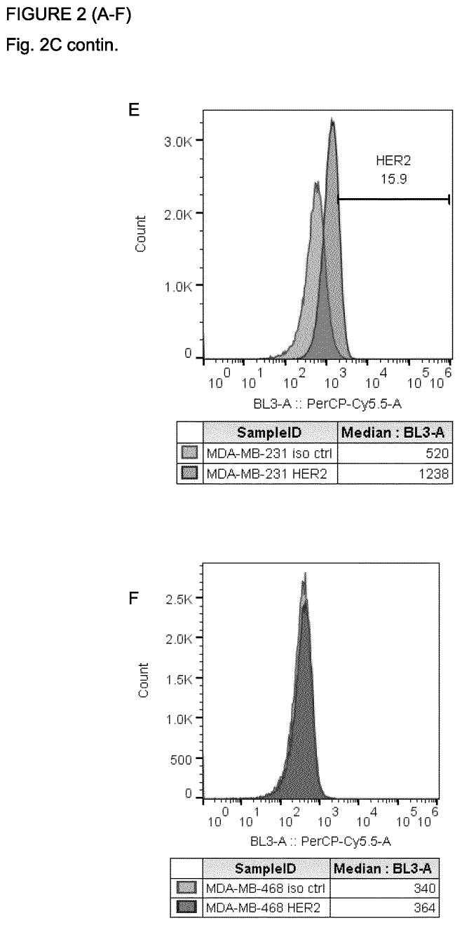

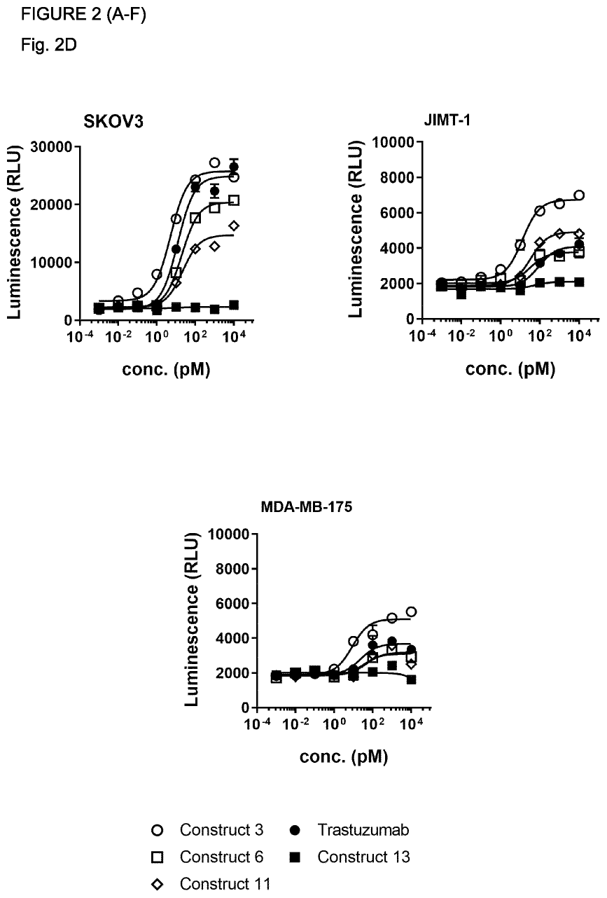

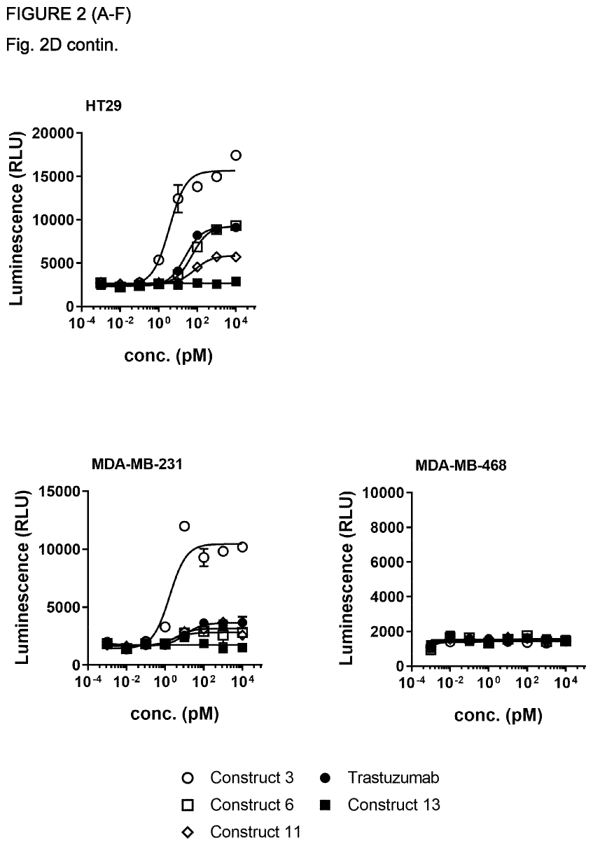

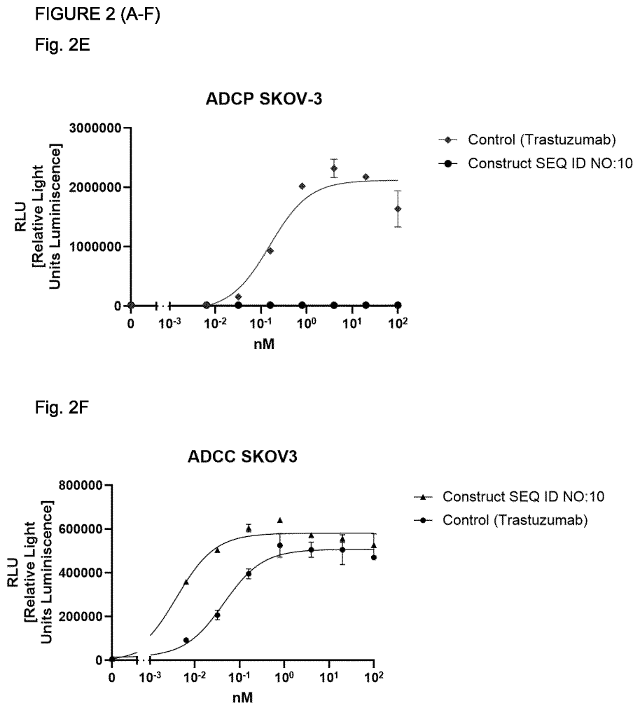

CD16a binders according to the invention were formatted as bi-specific (two-domains or 2D) NK cell engager DARPins having binding specificity for CD16a and HER2 as further detailed in Tables 3 (section 3.1) and 4 (section 3.2). All constructs in Tables 3 and 4 further comprise a N-terminal His Tag of SEQ ID NO: 69. The HER2 binder of SEQ ID NO: 9 binds the membrane distal domain II of HER2, whereas the one of SEQ ID NO: 8 binds to the membrane proximal domain IV of HER2, hence these binders are specific for different HER2 epitopes (U.S. Pat. No. 10,370,414 B2). The activity of such NK-cell engagers was tested in an antibody-dependent cellular cytotoxicity (ADCC) assay The effector cells used in this assay were Jurkat-NFAT-CD16a Reporter cells (same as in Example 2), which express the Lucia luciferase reporter gene upon binding of their Fc receptor CD16a. The extent of induced ADCC can therefore be quantified by a luminescent readout. The target cells used in the assay were SKOV3 tumor cells (DSMZ) which express the HER2 receptor. A schematic view of this assay is represented in FIG. 2A.

### 3.1 First Experiment

SKOV3 target cells were plated in 96 well U-bottom plates a day prior to the assay at a density of 10000 cells per well in DMEM medium supplemented with 10% FBS and were incubated overnight at 37° C. Serially diluted test constructs were added to the SKOV3 cells, in assay medium (IMDM+10% FBS). Trastuzumab (Roche) was used as a reference molecule and monovalent HER2 binders (Construct 12 and Construct 13) were used as negative controls. Finally, Jurkat-Lucia NFAT-CD16a Reporter cells were added at a concentration of 50000 cells per well, so as to achieve an E:T ratio of 5:1. The assay incubation duration was 5 h after which the assay plates were centrifuged at 300 g for 5 min. 50 μL supernatants from each plates were added onto Luminescence plates (Corning 3610) and mixed together with equal volume (50 μL) of QuantiLuc substrate which was prepared as per the manufacturer's guidelines. The resulting signal was measured as luminescence at Tecan plate reader (Tecan Infinite M1000 Pro Reader). A control measurement in which the constructs were incubated with the reporter cells but without tumor cells was also performed. Luminescence values are shown with a standard non-linear regression three parameter logistic model fit in FIG. 2B.

TABLE 3

| Construct | | | HER2 | |
|---|---|---|---|---|
| No. | Format | CD16a binder | binder | Linker |
| 3 (SEQ ID | 2D (CD16a × | SEQ ID NO: 2 | SEQ ID | PT linker (SEQ |
| NO: 13) | HER2) | (DARPin #2) | NO: 9 | ID NO: 70) |
| 4 (SEQ ID | 2D (HER2 × | SEQ ID NO: 3 | SEQ ID | PT linker (SEQ |
| NO: 15) | CD16a) | (DARPin #3) | NO: 9 | ID NO: 70) |
| 5 (SEQ ID | 2D (HER2 × | SEQ ID NO: 2 | SEQ ID | PT linker (SEQ |
| NO: 11) | CD16a) | (DARPin #2) | NO: 9 | ID NO: 70) |
| 6 (SEQ ID | 2D (CD16a × | SEQ ID NO: 3 | SEQ ID | PT linker (SEQ |
| NO: 17) | HER2) | (DARPin #3) | NO: 9 | ID NO: 70) |
| 7 (SEQ ID | 2D (HER2 × | SEQ ID NO: 2 | SEQ ID | PT linker (SEQ |
| NO: 10) | CD16a) | (DARPin #2) | NO: 8 | ID NO: 70) |
| 8 (SEQ ID | 2D (CD16a × | SEQ ID NO: 2 | SEQ ID | PT linker (SEQ |
| NO: 12) | HER2) | (DARPin #2) | NO: 8 | ID NO: 70) |
| 9 (SEQ ID | 2D (HER2 × | SEQ ID NO: 3 | SEQ ID | PT linker (SEQ |
| NO: 14) | CD16a) | (DARPin #3) | NO: 8 | ID NO: 70) |
| 10 (SEQ ID | 2D (CD16a × | SEQ ID NO: 3 | SEQ ID | PT linker (SEQ |
| NO: 16) | HER2) | (DARPin #3) | NO: 8 | ID NO: 70) |
| 11 (SEQ ID | 2D (CD16a × | SEQ ID NO: 1 | SEQ ID | PT linker (SEQ |
| NO: 18) | HER2) | (DARPin #1) | NO: 9 | ID NO: 70) |
| 12 | 1D (HER2) | NA | SEQ ID | NA |
| | | | NO: 8 | |
| 13 | 1D (HER2) | NA | SEQ ID | NA |
| | | | NO: 9 | |

Constructs in both formats (i.e. CD16a×HER2 and HER2×CD16a) showed a dose-dependent response (e.g. Construct 7 vs Construct 8) and by comparing constructs comprising HER2 binding domains with different HER2 epitope specificity, it can be concluded that binding to a particular epitope is not critical for activation (e.g Construct 7 vs Construct 5). EC50 values are shown in Table 3a.

TABLE 3a

| Construct No. | EC50 (pM) |
|---|---|
| 3 | 0.19 |
| 4 | 0.70 |
| 5 | 0.39 |
| 6 | 3.5 |
| 7 | 0.37 |
| 8 | 0.24 |
| 9 | 1.6 |
| 10 | 24 |
| 11 | 4.1 |
| Trastuzumab | 2.4 |
| (mean) | |

### 3.2 Second Experiment

A further ADCC assay was performed similarly as in section 3.1, except that various HER2-expressing cells with different HER2 expression profiles were used as target tumor cells in conjunction with the Jurkat-Lucia NFAT-CD16a Reporter cells. The expression of HER2 was measured by flow cytometry and is shown for each cell type in FIG. 2C. Constructs tested in this assay are shown in Table 4a. In brief, different in vitro cultured HER2 expressing cell lines were washed 1× with PBS, centrifuged at 300 g, supernatant was discarded and cells were resuspended in PBS at 10 mio/ml. Live/dead aqua stain was added at a 1:1000 dilution, incubated for 20 min at 4° C. and washed again with PBS+2% FBS. After centrifugation, cells were resuspended in PBS+2% FBS at 10 mio/ml. Cell suspension were distributed into 96-well round bottom plates at 100 μl/well and 100 μL/well of 1:20-diluted anti-HER2 antibody (Anti-Her2 PerCP/Cy5.5, BioLegend #324416 or Isotype ctrl PerCP/Cy5.5 antibody) were added. Following 30 min of incubation at 4° C., cells were washed 2× with PBS+2% FBS and resuspended in 200 μL/well 1:10-diluted cell fixation buffer (BD, #554655). FACS analysis was performed using a Attune Nxt Flow Cytometer. Shown are histograms of the PerCP/Cy5.5 fluorescence intensity signal of the anti-HER2 vs isotype control antibodies. Based on these measures, the following semi-quantitative HER2 expression was established:
SKOV3: ++++
JIMT-1: +++
MDA-MB-175: ++
HT29: +
MDA-MB-231: (+)
MDA-MB-468: −

The resulting signals in the ADCC assay using the different HER2-expressing cell lines were measured as in section 3.1 but after 24 hours after incubation instead of 5 hours, and are shown in FIG. 2D.

TABLE 4a

| Construct | | | HER2 | |
|---|---|---|---|---|
| No. | Format | CD16a binder | binder | Linker |
| 3 (SEQ ID | 2D (CD16a × | SEQ ID NO: 2 | SEQ ID | PT linker (SEQ |
| NO: 13) | HER2) | (DARPin #2) | NO: 9 | ID NO: 70) |
| 6 (SEQ ID | 2D (CD16a × | SEQ ID NO: 3 | SEQ ID | PT linker (SEQ |
| NO: 17) | HER2) | (DARPin #3) | NO: 9 | ID NO: 70) |
| 11 (SEQ ID | 2D (CD16a × | SEQ ID NO: 1 | SEQ ID | PT linker (SEQ |
| NO: 18) | HER2) | (DARPin #1) | NO: 9 | ID NO: 70) |
| 13 | 1D (HER2) | NA | SEQ ID | NA |
| | | | NO: 9 | |

### B. Functional Testing of CD16a-Specific DARPins in ADCP Reporter Cell Assay

The activity of an NK-cell engager with SEQ ID NO: 10 was tested in an antibody-dependent cellular phagocytosis (ADCP) reporter assay (Invivogen jktl-nfat-cd32). The effector cells used in this assay were Jurkat-NFAT-CD32a reporter cells, which express the Lucia luciferase reporter gene upon binding of their Fc receptor CD32a. The extent of induced ADCP can therefore be quantified by a luminescent readout. The target cells used in the assay were SKOV3 tumor cells (DSMZ) which express the HER2 receptor.

SKOV3 target cells were plated in 96 well U-bottom plates a day prior to the assay at a density of 100,000 cells per well in DMEM medium supplemented with 10% FBS and were incubated overnight at 37° C. Serially diluted test constructs were added to the SKOV3 cells, in assay medium (IMDM+10% FBS). Trastuzumab (Roche) was used as reference molecule. Finally, Jurkat-Lucia NFAT-CD32a Reporter cells were added at a concentration of 100,000 cells per well, so as to achieve an E:T ratio of 1:1. The assay incubation duration was 24 h after which the assay plates were centrifuged at 300 g for 5 min. 50 μL supernatants from each plates were added onto luminescence plates (Corning 3610) and mixed together with equal volume (50 μL) of QuantiLuc substrate which was prepared as per the manufacturer's guidelines. The resulting signal was measured as luminescence at Tecan plate reader (Tecan Infinite M1000 Pro Reader. As reference the same construct was tested in the ADCC reporter assay described in section 3.1 keeping the E:T ratio of 1:1 and 24 hours incubation time and Jurkat-Lucia NFAT-CD16a as reporter cells). Luminescence values of both experiments are shown with a standard non-linear regression three parameter logistic model fit in FIGS. 2E and 2F. The results obtained confirm, as shown in FIGS. 2E and 2F, that the tested NK cell engager exclusively mediates ADCC activity through CD16a engagement but not ADCP activity through CD32a engagement-namely the tested construct does not bind to CD32a expressed on the reporter cells (FIG. 2E) but to CD16a (FIG. 2F).

TABLE 4b

| | EC50 ADCP reporter assay | EC50 ADCC reporter assay |
|---|---|---|
| Construct SEQ | (CD32a) | (CD16a) |
| ID NO | (pM) | (pM) |
| SEQ ID NO:10 | NA | 4 |
| Trastuzumab | 160 | 44 |

### Example 4: CD16a-Specific DARPins Activate Primary NK Cells

Activation of human NK-cells by CD16a binders according to the invention formatted as bi-specific (two-domains or 2D) NK cell engagers was tested. Primary NK-cells were isolated from 50 mL whole blood (buffy coat) of a healthy donor through a CD56+ positive selection using a CD56 MicroBead Kit (Miltenyi) and following the manufacturer's protocol. HER2-expressing SKOV3 cells were used as target cells. A tumor to effector cells ratio of 5:1 was used, and readout was performed after 24 hours incubation at 37° C. The tested binders are shown in Table 5. All constructs in Table 5 also further comprise a N-terminal His Tag of SEQ ID NO: 69.

TABLE 5

| Construct | | | HER2 | |
|---|---|---|---|---|
| No. | Format | CD16a binder | binder | Linker |
| 3 (SEQ ID | 2D (CD16a × | SEQ ID NO: 2 | SEQ ID | PT linker (SEQ |
| NO: 13) | HER2) | (DARPin #2) | NO: 9 | ID NO: 70) |
| 4 (SEQ ID | 2D (HER2 × | SEQ ID NO: 3 | SEQ ID | PT linker (SEQ |
| NO: 15) | CD16a) | (DARPin #3) | NO: 9 | ID NO: 70) |
| 13 | 1D (HER2) | NA | SEQ ID | NA |
| | | | NO: 9 | |

### 4.1 IFNγ and Granzyme B Secretion

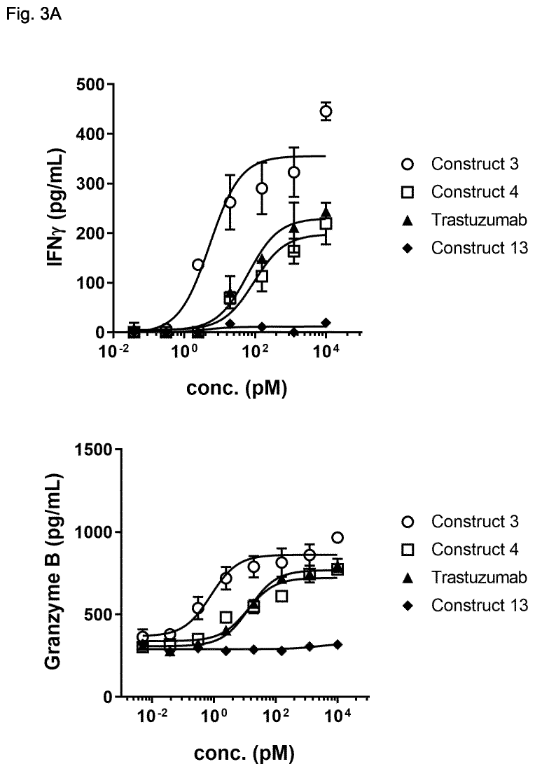

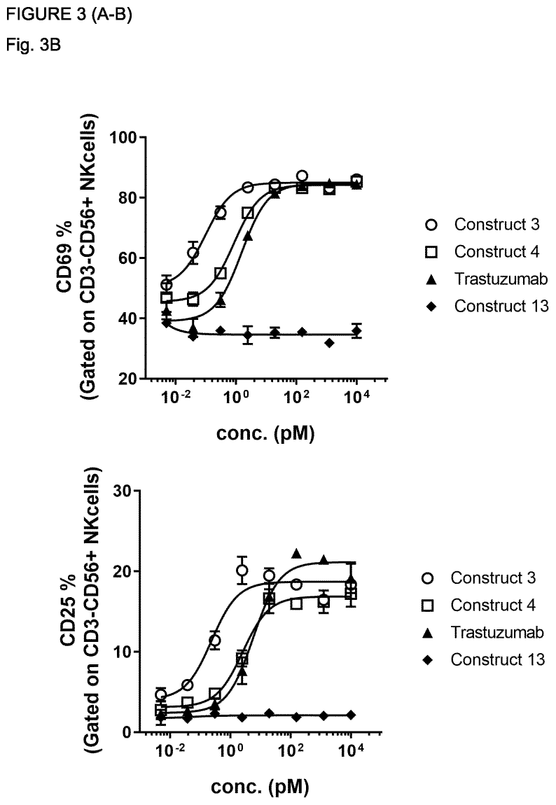

IFNγ and Granzyme B levels were measured by ELISA (IFNγ ELISA development Kit from Peprotech (900-K27) and Human Granzyme B Duoset ELISA from RnD (DY2906-05)) following the manufacturer's instructions. For each experiment, 50 μL of supernatant (undiluted) was transferred to 96-well flat bottom pre-coated ELISA plates and after incubation with the samples and the respective secondary antibodies substrate followed by stop solution were added to the wells and measurement was done on a TECAN reader (OD 405-620 nm for IFNγ and 420 nm-620 nm for Granzyme B). Results are shown in FIG. 3A. EC50 values are shown in Table 5a.

TABLE 5a

| | EC50 (pM) IFNγ | |
|---|---|---|
| Construct No. | assay | EC50 (pM) Granzyme B assay |
| 3 | 5.2 | 0.79 |
| 4 | 93 | 13 |
| Trastuzumab | 62 | 14 |

### 4.2 Activation Markers (CD69 and CD25)

50000 isolated NK effector cells and 10000 HER2-expressing SKOV3 cells per well were co-incubated (E:T ratio 5:1) with serial dilutions of the selected Constructs in duplicates for 24 hours at 37° C. After 24 hours, cells were washed and stained with 1:1'000 Live/Dead-aqua (Thermo Fisher) for 20 minutes at 4° C. Cells were washed once with PBS supplemented with 10% FBS and incubated with the staining mix containing 1:100 dilution of mouse anti-human CD56 APC (eBiosciences), mouse anti-human CD3 FITC, mouse anti-human CD16 BV421 (BD Biosciences), mouse anti-human Nkp46 PE (BD Biosciences), mouse anti-human CD25 PErCP-Cy5.5 (eBiosciences) and 1:200 dilution of mouse anti-human CD69 PE-Cy7 (BD Biosciences). After washing and fixation, cells were analyzed on Attune NxT (ThermoFisher) machine. NK cell activation was assessed by measuring CD25+ and CD69+ cells on Live/Dead-negative and CD3− CD56+ gated NK cells. FACS data was analyzed using FlowJo software and data was plotted using GraphPad Prism 8 (3-PL-fit). Results are shown in FIG. 3B. EC50 values are shown in Table 5b.

TABLE 5b

| Construct No. | EC50 (pM) CD69 marker | EC50 (pM) CD25 marker |
|---|---|---|
| 3 | 0.098 | 0.24 |
| 4 | 0.86 | 2.6 |
| Trastuzumab | 1.5 | 5.8 |

### Example 5: Tumor Cell Killing Assay, Primary NK-Cell and HER2 Expressing Tumor Cells

Tumor cell killing potency of CD16a binders according to the invention formatted as bi-specific (two-domains or 2D) NK cell engagers was tested. Primary NK-cells were prepared as described in Example 4. In a first experiment, HER2-expressing SKOV3 tumor cells were used (section 5.1), and in a second experiment, cell lines with variable HER2-expressing profiles (as in example 3.2) were tested as target cells. Potency was measured as LDH release using the Cytotoxicity Detection Kit (LDH) Roche/Sigma 11644793001.

### 5.1 First Experiment

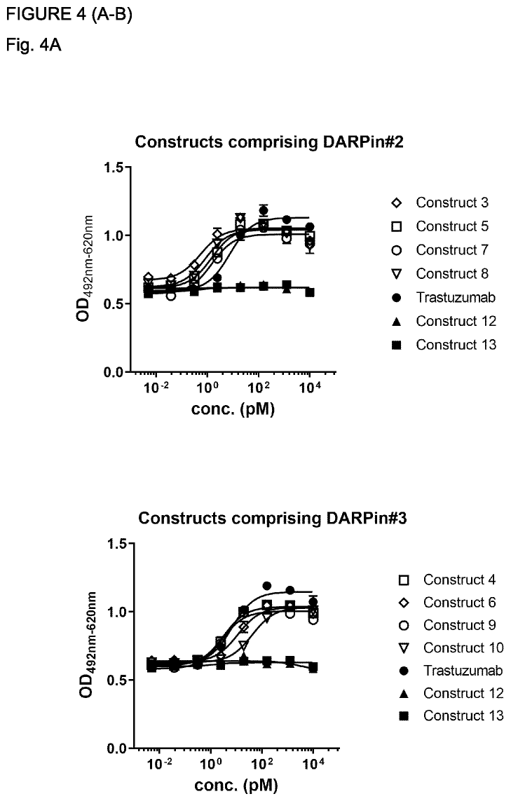

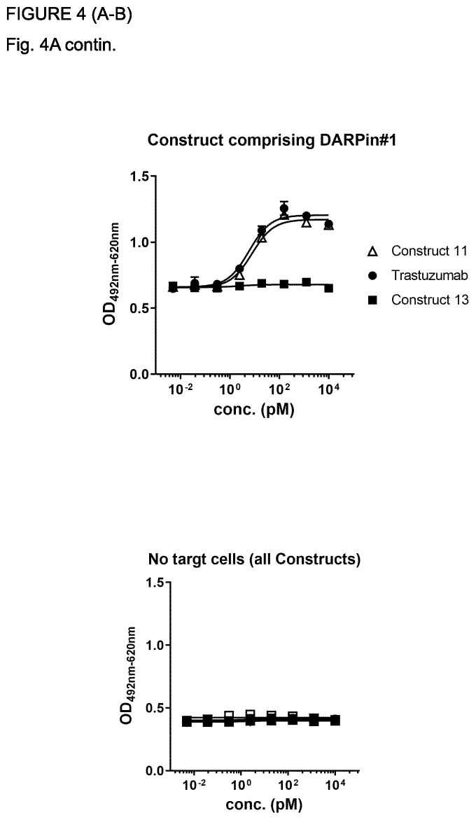

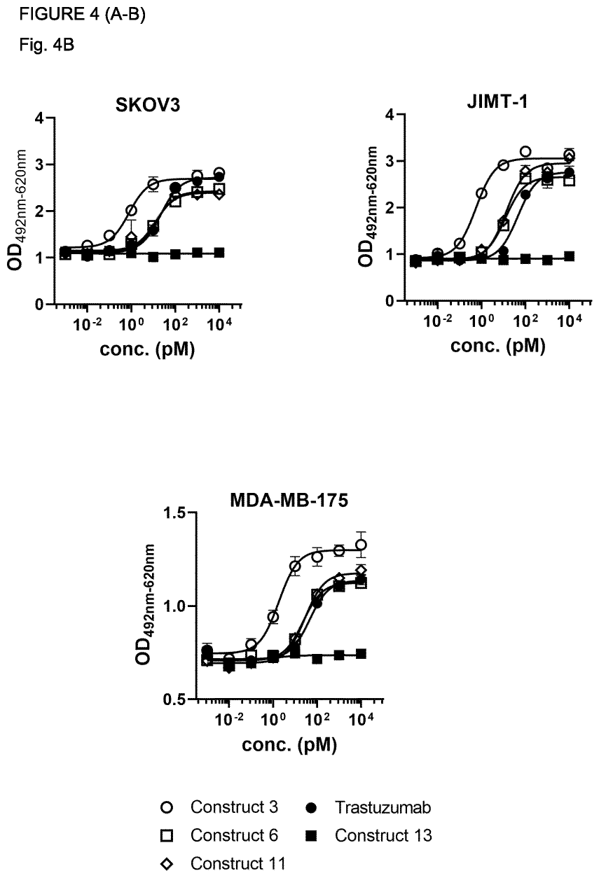

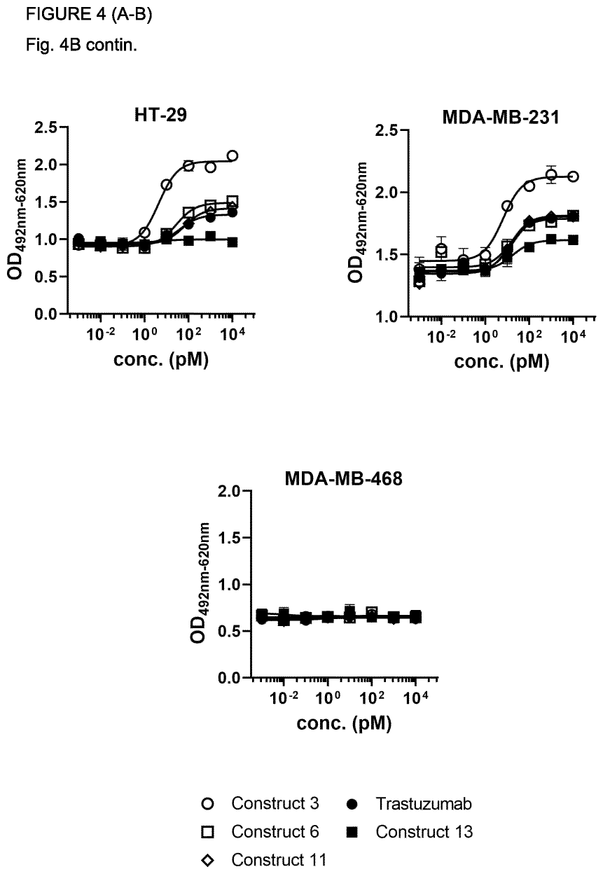

The same constructs as in section 3.1 (see Table 3) were tested following the same experimental procedure described in Example 4.2. For the cytotoxicity determination, 100 μL of supernatant were transferred to a 96-well flat bottom ELISA plate and the assay was performed following the manufacturer's instructions. A control measurement in which the constructs were incubated with the NK cells but without tumor cells was also performed. Results are shown in FIG. 4A. EC50 values are shown in Table 5c.

TABLE 5c

| Construct No. | EC50 (pM) |
|---|---|
| 3 | 0.54 |
| 4 | 3.2 |
| 5 | 1.5 |
| 6 | 11 |
| 7 | 1.5 |
| 8 | 0.86 |
| 9 | 2.7 |
| 10 | 41 |
| 11 | 7.8 |
| Trastuzumab (mean) | 6.7 |

### 5.2 Second Experiment

The same constructs as in section 3.2 (see Table 4a) were tested following the same experimental procedure as described in Example 4.2 but using the different cell lines as target cells. For the cytotoxicity determination, 100 μL of supernatant were transferred to a 96-well flat bottom ELISA plate and the assay was performed following the manufacturer's instructions. Results are shown in FIG. 4B. EC50 values are shown in Table 5d.

TABLE 5d

| | EC50 (pM) | | | | |
|---|---|---|---|---|---|
| Construct | | | MDA-MB- | | |
| No. | SKOV3 | JIMT-1 | 175 | HT-29 | MDA-MB-231 |
| 3 | 0.78 | 0.55 | 1.82 | 4.37 | 6.40 |
| 6 | 12.86 | 12.15 | 23.13 | 27.55 | 14.00 |
| 11 | 12.70 | 13.34 | 28.14 | 64.62 | 13.20 |
| Trastuzumab | 21.74 | 45.05 | 45.51 | 41.77 | 16.43 |

Both experiments 5.1 and 5.2 therefore support that the tested construct comprising ankyrin repeat proteins with binding specificity for CD16a according to the invention and a HER2 binding ankyrin repeat domain can induce activation of NK cells and target tumor cell killing.

### Example 6: Determination of Dissociation Constants (K_{D}) of Recombinant Ankyrin Repeat Proteins with Binding Specificity for CD16a by Surface Plasmon Resonance (SPR) Analysis

The binding affinity of Construct No. 1 comprising DARPin #2 (Table 1) on recombinant CD16a target was analysed by SPR, measurement was performed according to standard procedures known to the person skilled in the art.

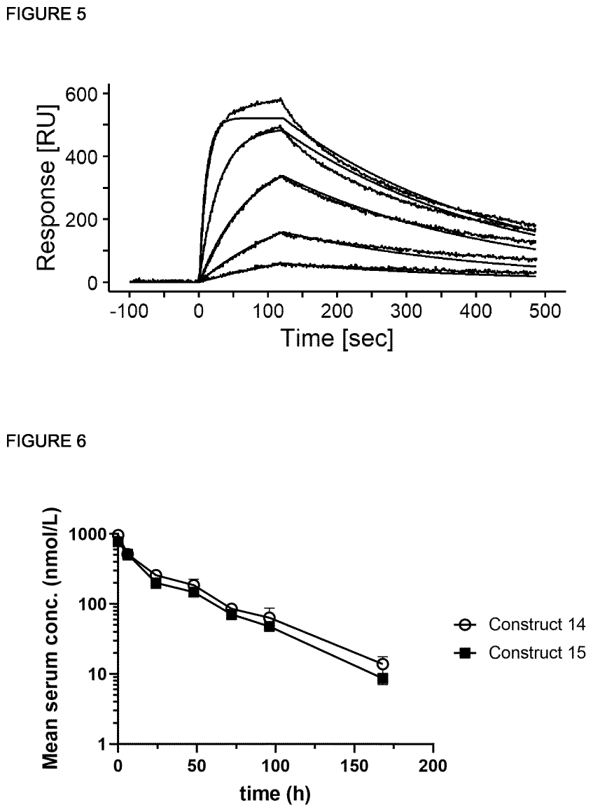

SPR measurements were performed using a ProteOn XPR36 instrument (BioRad). The running buffer was PBS pH 7.4 containing 0.005% Tween 20® (PBST). Biotinylated CD16aV (bio-CD16aV) target was immobilized on a NLC chip (BioRad) to a level of about 3200 RU. The interactions of purified CD16a-specific ankyrin construct with bio-CD16aV were measured by injecting the construct at 100 nM (and 1:3 dilutions) with an association of 120 s and dissociation of 300 s using a constant flow of 100 μl/min. The target was regenerated between the individual measurements using 16 mM H3PO4. The signals were double referenced against the running buffer (PBST) treated control lanes. The K_{D} was calculated with a 1:1 Langmuir model fitting from the estimated on- and off-rates using standard procedures known to the person skilled in the art. The K_{D} value obtained in for Construct No. 1 is 3.30 nM, and corresponding SPR curves are shown in FIG. 5.

### Example 7: Pharmacokinetic Analysis of CD16a-Specific Ankyrin Repeat Proteins in Female BALB/c Mice

To determine whether a CD16a-specific ankyrin repeat domain of the invention can have an appropriate serum half-life in vivo for it to be useful for the development of therapeutic agents, the pharmacokinetic profiles of DARPin #2 and DARPin #3 were analysed in mice. For that, DARPin constructs were subcloned and expressed as described above into derivatives of the pQE30 (Qiagen) expression vector, resulting in expression constructs encoding an N-terminal His-tag (SEQ ID NO: 69), a human serum albumin (HSA) binding ankyrin repeat domain (SEQ ID NO: 24) for half-life extension, a peptide linker (SEQ ID NO: 70), and at the C-terminal end one of the CD16a-specific binding domains. The constructs are summarized in Table 6.

TABLE 6

| Construct | | | HSA | |
|---|---|---|---|---|
| No. | Format | CD16a binder | binder | Linker |
| 14 (SEQ ID | 2D (HSA × | SEQ ID NO: 2 | SEQ ID | PT linker (SEQ |
| NO: 22) | CD16a) | (DARPin #2) | NO: 24 | ID NO: 70) |
| 15 (SEQ ID | 2D (HSA × | SEQ ID NO: 3 | SEQ ID | PT linker (SEQ |
| NO: 23) | CD16a) | (DARPin #3) | NO: 24 | ID NO: 70) |

### In Vivo Administration and Sample Collection

Constructs Nos. 14 and 15 as described above, were administered as a single intravenous bolus injection into the tail vein of 6 mice for each ankyrin repeat fusion protein. The target dose level was 1 mg/kg with an application volume of 5 mL/kg. Constructs were formulated in phosphate-buffered saline (PBS) solution.

Mice were split into 2 groups with equal numbers of animals. Four serum samples were collected from each mouse. Blood samples for pharmacokinetic investigations were collected from the saphenous vein at 5 min, 6 h, 24 h, 48 h, 72 h, 96 h and 168 h post compound administration. Blood was kept at room temperature to allow clotting followed by centrifugation and collection of serum.

### Bioanalytics by ELISA to Measure Ankyrin Repeat Proteins in Serum Samples

100 μl per well of 10 nM polyclonal goat anti-rabbit IgG antibody (Ab18) in PBS was coated onto a NUNC Maxisorb ELISA plate overnight at 4° C. After washing with 300 μl PBST (PBS supplemented with 0.1% Tween20) per well three times, the wells were blocked with 300 μl PBST supplemented with 0.25% Casein (PBST-C) for 1 h at room temperature (RT) on a Heidolph Titramax 1000 shaker (450 rpm). Plates were washed as described above. 100 μl 5 nM rabbit anti-DARPin 1-1-1 antibody in PBST-C was added and the plates were incubated at RT (22° C.) with orbital shaking (450 rpm) for 1 h. Plates were washed as described above. 100 μl of diluted serum samples (1:20-1:312500 in 1:5 dilution steps) or ankyrin repeat protein standard curve samples (0 and 50-0.0008 nM in 1:3 dilution steps) were applied for 2 h, at RT, shaking at 450 rpm. Plates were washed as described above.

Wells were then incubated with 100 μl murine anti-RGS-His-HRP IgG (Ab06, 1:2000 in PBST-C) and incubated for 1 h, at RT, 450 rpm. Plates were washed as described above. The ELISA was developed using 100 μl/well TMB substrate solution for 10 minutes and stopped by the addition of 100 μl 1 mol/L H₂SO₄. The difference between the absorbance at 450 nm and the absorbance at 620 nm was calculated. Samples were measured in duplicate on two different plates.

### Pharmacokinetic Analysis

Pharmacokinetic data analysis was performed using Version 8.0 of the WinNonlin program as part of Phoenix 64, Pharsight, North Carolina. Calculation of the pharmacokinetic parameters based on the mean concentration-time data of the animals dosed via intravenous bolus injection was performed with non-compartmental analysis (NCA model 200-202, IV bolus, linear trapezoidal linear interpolation). Pharmacokinetic parameters are calculated, such as the following:
AUCinf, AUClast, AUC_% extrapol, Cmax, Tmax, CI_pred, Vss_pred, t½.

Maximum serum concentrations (Cmax) and the times of their occurrence (Tmax) were obtained directly from the serum concentration-time profiles. The area under the serum concentration-time curve (AUCinf) was determined by the linear trapezoidal formula up to the last sampling point (Tlast) and extrapolation to infinity assuming mono-exponential decrease of the terminal phase. The extrapolation up to infinity was performed using Clast/λz, where λz denotes the terminal rate constant estimated by log linear regression and Clast denotes the concentration estimated at Tlast by means of the terminal log-linear regression. Total serum clearance (CI_pred) and the apparent terminal half-life were calculated as follows: CI_pred=i.v. dose/AUCinf and t½=ln 2/λz. The steady-state volume of distribution Vss was determined by: Vss=i.v. dose·AUMCinf/(AUCinf)2. AUMCinf denotes the total area under the first moment of drug concentration-time curve extrapolated to infinity using the same extrapolation procedure as described for calculation of AUCinf. To calculate PK parameters based on concentrations given in nmol/L dose, values given as mg/kg were converted to nmol/kg by using the molecular weight of the ankyrin repeat proteins. Evolution of the measured mean serum concentration of the injected Constructs is shown in FIG. 6.

Table 7 shows the approximate predicted half-lives of DARPin #2 and DARPin #3, as formatted with a human serum albumin-specific ankyrin repeat domain (Constructs No. 14 and 15 respectively).

TABLE 7
Half-life (t1/2) of two exemplary HSA ×
CD16a-specific ankyrin repeat proteins

| Parameter | unit | Construct No. 14 | Construct No. 15 |
|---|---|---|---|
| HL_Lambda_z (half- life) | h | ~32.1 | ~28.9 |

In conclusion, CD16a-specific ankyrin repeat domains of the invention can be combined with a half-life extending moiety, such as, e.g., a serum albumin-specific binding domain, to achieve an appropriate serum half-life in vivo for them to be useful for the development of therapeutic agents.

### Example 8: Cross-Reactivity to Human and Cynomolgus Monkey CD16a Target

Cross-reactivity of selected CD16a-specific ankyrin repeat domains of the invention (i.e. DARPin #2 of SEQ ID NO: 2 and DARPin #7 of SEQ ID NO: 7), each further comprising an N-terminal His Tag of SEQ ID NO: 69) to human and cyno CD16a target was tested by SPR.

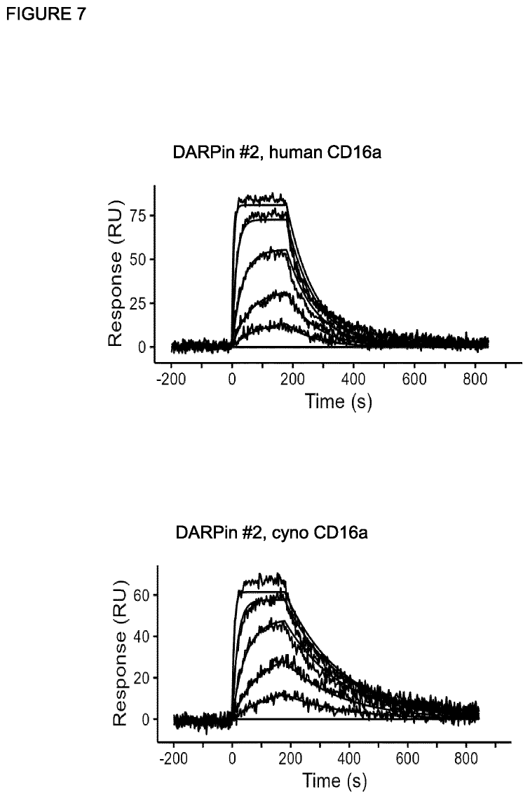

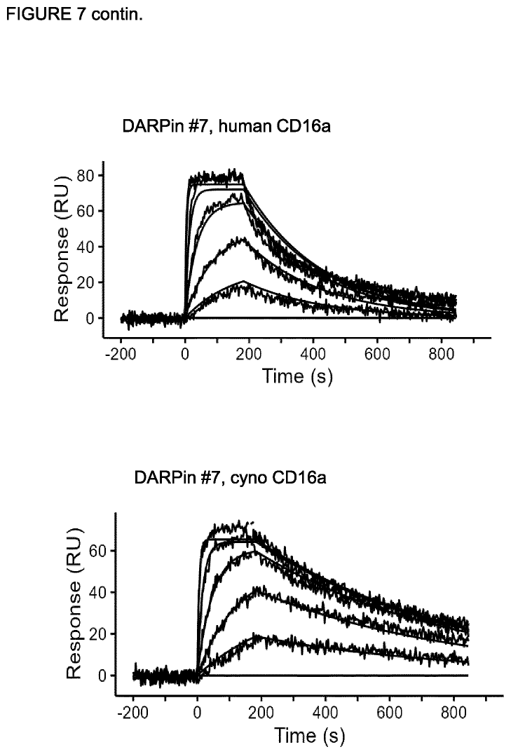

SPR measurements were performed using a ProteOn XPR36 instrument (BioRad). Each of biotinylated human target (bio-CD16aV of SEQ ID NO: 72) and biotinylated cyno target (bio-cCD16 of SEQ ID NO: 76) were immobilized on a neutravidin chip (NAHLC200M, Xantec ProteOn Sensor Chip) to a level of about 320 RU. The interactions of purified CD16a-specific ankyrin construct with bio-CD16aV or bio-cCD16 were measured by injecting the DARPins at 100 nM (and 1:3 dilutions) with an association of 180 s and dissociation of 600 s using a constant flow of 30 μl/min. The target was regenerated between the individual measurements using 31 mM H3PO4. The signals were double referenced against the running buffer (PBST) treated control lanes. The K_{D} were calculated with a 1:1 Langmuir model fitting from the estimated on- and off-rates using standard procedures known to the person skilled in the art. The K_{D} values obtained are shown in Table 8 and corresponding SPR curves are shown in FIG. 7.

TABLE 8

| Sample | Human CD16a, K_{D} in [M] | Cyno CD16a, K_{D} in [M] |
|---|---|---|
| DARPin #2 | 5.94E−09 | 3.41E−09 |
| DARPin #7 | 1.98E−09 | 9.04E−10 |

Both DARPins showed comparable K_{D} values for cyno CD16a and human CD16a targets.

### Example 9: CD16a Allelic Variants Specificity

DARPin #7 was formatted as bispecific (two-domains or 2D) immune cell engager construct (i.e. Construct 25 of SEQ ID NO: 94) having binding specificity for CD16a and CD117 (or cKit), by linking a CD117-specific ankyrin repeat domain of SEQ ID NO: 84 to the N-terminal side of DARPin #7.

Binding of Construct 25 to different CD16 allelic variants was tested, using three different Jurkat target cell lines expressing CD16aV, CD16aF or CD16b. Stable expression of the respective CD16 allelic variants on the cell surface was confirmed by flow cytometry. Experimental conditions were as described in Example 2. As shown by the measured EC50 values (Table 9), Construct 25 binds both CD16aV and CD16aF expressing cells, and does not bind to cells expressing CD16b, supporting the target specificity of the binders of the invention.

TABLE 9

| | EC50 Jurkat | EC50 Jurkat | EC50 Jurkat |
|---|---|---|---|
| Construct | CD16aV | CD16aF | CD16b |
| Construct 25 | 25.34 nM | 23.20 nM | Not detectable |
| (SEQ ID NO: 94) | | | |

Construct 25 further comprised an N-terminal His tag (SEQ ID NO: 69) for ease of purification.

### Example 10: Functional Assessment of Selected CD16a-CD117-Bispecific DARPins

### 10.1 DARPin-Dependent Cellular Cytotoxicity (DDCC)

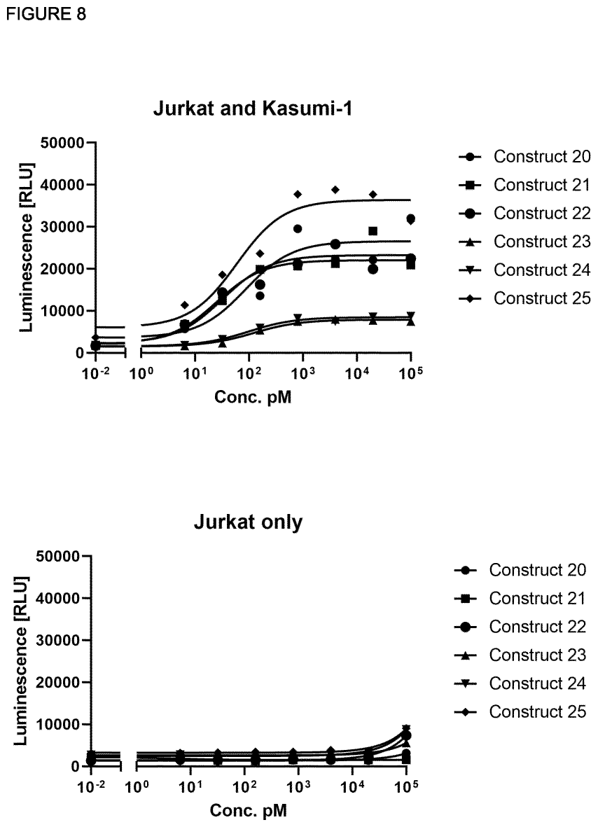

Selected CD16a-specific ankyrin repeat domains were formatted as bispecific (two-domains or 2D) immune cell engager DARPins having binding specificity for CD16a and CD117 (or cKit) as further detailed in Table 10. The cytotoxic activity of these engagers was tested in a DDCC reporter assay using Jurkat-NFAT-CD16a reporter cells. Upon binding of DARPin to CD16a, these reporter cells express the Lucia luciferase reporter gene. The extent of induced DDCC can therefore be quantified by a luminescent readout. Kasumi-1 cells, which express the CD117 receptor, were used as target cells in this assay. In order to assess the levels of specific DDCC activity, Jurkat cells in the absence of target cells were used as a control. Briefly, Kasumi-1 target cells were plated in 96 well U-bottom plates one day prior to the assay at a density of 100,000 cells per well in DMEM medium supplemented with 10% FBS and were incubated overnight at 37° C. Constructs serially diluted in assay medium were added to the Kasumi-1 cells. Finally, Jurkat-Lucia NFAT-CD16a Reporter cells were added at a concentration of 100,000 cells per well, so as to achieve an E:T ratio of 1:1. The assay incubation duration was 24 h after which the assay plates were centrifuged at 300 g for 5 min. 50 μL of the supernatant from each plate was added onto luminescence plates (Corning 3610) and mixed together with an equal volume (50 μL) of QuantiLuc substrate which was prepared as per the manufacturer's guidelines. The resulting signal was measured as luminescence using a Tecan plate reader (Tecan Infinite M1000 Pro Reader). Luminescence values are shown with a standard non-linear regression three parameter logistic model fit. As shown in FIG. 8 and Table 10, all tested constructs show specific and dose dependent cytotoxicity. The maximum levels of cytotoxicity observed correlated with the level of binding observed for the different CD117-CD16a specific DARPins tested.

TABLE 10

| Construct | | CD117 | | EC50 | Top |
|---|---|---|---|---|---|
| No. | Format | binder | CD16a binder | (pM) | (RLU) |
| 20 | CD117 × | SEQ ID | SEQ ID NO: 2 | 86.2 | 26541 |
| (SEQ ID | CD16a | NO: 84 | (DARPin #2) | | |
| NO: 89) | | | | | |
| 21 | CD117 × | SEQ ID | SEQ ID NO: 2 | 32.3 | 23236 |
| (SEQ ID | CD16a | NO: 85 | (DARPin #2) | | |
| NO: 90) | | | | | |
| 22 | CD117 × | SEQ ID | SEQ ID NO: 2 | 25.2 | 22034 |
| (SEQ ID | CD16a | NO: 86 | (DARPin #2) | | |
| NO: 91) | | | | | |
| 23 | CD117 × | SEQ ID | SEQ ID NO: 2 | 120.2 | 7871 |
| (SEQ ID | CD16a | NO: 87 | (DARPin #2) | | |
| NO: 92) | | | | | |
| 24 | CD117 × | SEQ ID | SEQ ID NO: 2 | 96.1 | 8471 |
| (SEQ ID | CD16a | NO: 88 | (DARPin #2) | | |
| NO: 93) | | | | | |
| 25 | CD117 × | SEQ ID | SEQ ID NO: 7 | 59.5 | 36390 |
| (SEQ ID | CD16a | NO: 84 | (DARPin #7) | | |
| NO: 94) | | | | | |

The bispecific Constructs further comprised an N-terminal His tag (SEQ ID NO: 69) for ease of purification. CD117 and CD16a binding domains are connected by a PT linker with SEQ ID NO: 70.

### 10.2 NK Cell Degranulation

The ability of Constructs 20 to 24 to mediate activation of primary NK cells was further assessed by NK cell degranulation observed in the presence of target expressing cells. Peripheral Blood Mononuclear cells (PBMCs) were isolated from buffy coats from healthy donors using the human NK cell isolation kit (Miltenyi Biotec) according to manufacturer's instructions. After isolation, NK cells were kept overnight in medium containing human interleukin-2 (hlL-2) at 37° C. Next, Kasumi-1 target cells, NK cells, titrations of the tested proteins and the anti-CD107a BV421 antibody (Biolegend) were added to a 96 well plate and incubated for 2 hours at 37° C. A negative control two-domain construct comprising as first domain a non-binding DARPin and as second domain DARPin #2 (“Control Construct 1” of SEQ ID NO: 95) was also included. Cells were then centrifuged at 350 g for 4 min at 4° C. and washed with PBS, followed by cell resuspension with human Fc block (BD Biosciences) and incubation for 15 min at 4° C. After centrifugation, an antibody mix (CD56 BV605 BD and CD16 FITC Biolegend) was added to the cells in FACS buffer which were incubated for 30 min at 4° C. Cells were centrifuged, washed and stained with the Live Dead Zombie NIR (Biolegend) for 20 min at 4° C., then were washed and fixed/permeabilized with the FoxP3/Transcription Factor staining buffer set (Invitrogen) following the instructions provided by the manufacturer. Cells were finally analysed by flow cytometry in the Attune NxT device.

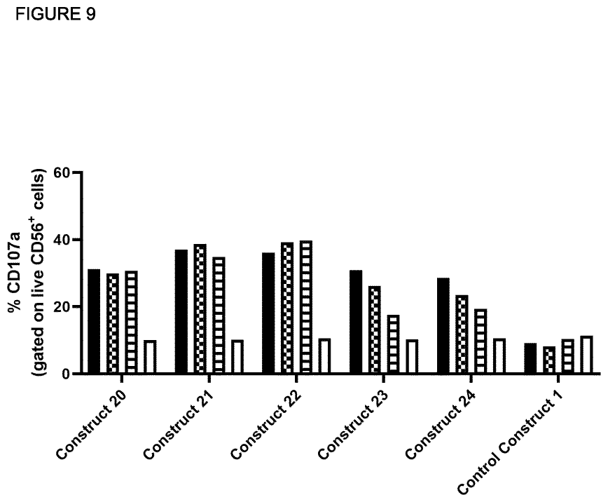

Live NK cells (CD56+) were gated and levels of degranulation were obtained by assessing the degranulation marker CD107a. FIG. 9 shows that all tested constructs mediated efficient NK cell degranulation through CD16a engagement.

### 10.3 Macrophage Induced Cellular Phagocytosis

The activity of bispecific Constructs 20 to 25 was further tested in a DARPin-dependent cellular phagocytosis (DDCP) assay by flow cytometry. Macrophages derived from monocytes isolated from cryopreserved Peripheral Blood Mononuclear cells (PBMCs) from healthy donors were used as effector cells. Kasumi-1 cells, which express CD117, were used as target cells. Briefly, monocytes were isolated from cryopreserved PBMCs using a CD14 ultrapure isolation kit (Miltenyi Biotec, 130-118-906), according to manufacturer's instructions. After isolation, monocytes were differentiated into MO-like macrophages by in vitro culture with M-CSF (Miltenyi Biotec, 130-093-866) in Cellgenix GMP DC medium for six days (final well concentration of 0.05 μg/ml). Macrophages were then harvested, washed and seeded overnight in an assay plate. The next day, Kasumi-1 target cells were harvested, labelled with a proliferation dye (Cell Trace TM Yellow cells, Thermofischer) according to the manufacturer's instructions and pre-incubated with the tested constructs, next to benchmark monoclonal antibody (anti-CD117 mAb) and a negative control IgG1. Kasumi-1 cells were then added on top of the pre-seeded macrophages at an effector to target ratio of 5:1 for 4 hours at 37° C. After co-culture, plates were centrifuged and cells were harvested using dissociation buffer (Cell dissociation buffer, Gibco, 13151014). Cells were washed and stained for viability (Zombie Aqua viability dye, Biolegend, 30 min at 4° C.). After a washing step, cells were incubated with Fc blocking reagent (Miltenyi Biotec, 130-059-901) for 5 min at 4° C. Cells were then centrifuged, washed twice and stained with an antibody mix (containing anti-CD11 b APC Miltenyi Biotec 130-110-554) for 30 min at 4° C. Cells were centrifuged and washed twice and 30 μl of fix buffer was added for 20 min at RT. Finally, cells were washed twice, resuspended in 150 μl of PBS+2 nM EDTA and acquired on the Attune NxT flow cytometer. The percentage of DDCP corresponds to the percentage of CD11b+CellTrace+ cells.

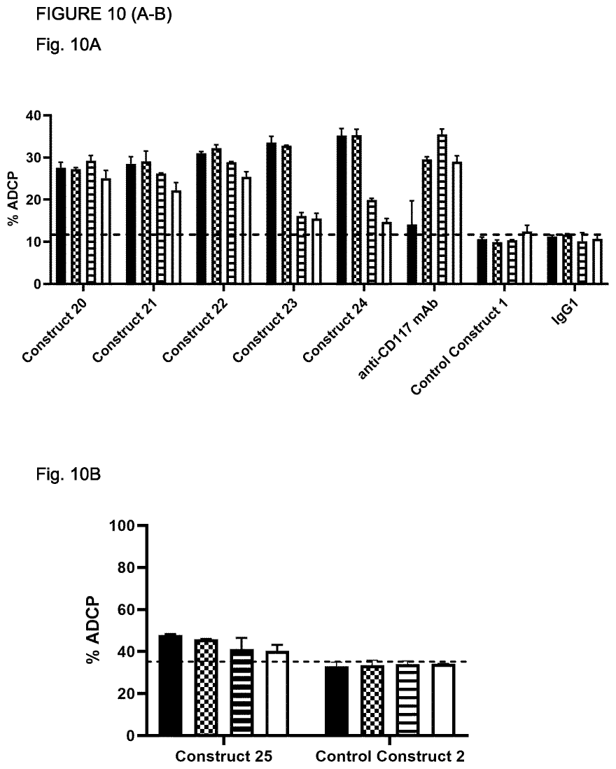

Two separate experiments were performed: Experiment 1 in which Constructs 20 to 24 and controls were applied at 10 nM, 1 nM, 0.1 nM and 0.01 nM (FIG. 10A), and Experiment 2 in which Construct 25 and control were applied at 1 nM, 0.2 nM, 0.04 nM and 0.008 nM (FIG. 10B). Indicated controls correspond to two-domain construct comprising as first domain a non-binding DARPin and as second domain DARPin #2 (“Control Construct 1” of SEQ ID NO: 95 used in Experiment 1) or DARPin #7 (“Control Construct 2” of SEQ ID NO: 96 used in Experiment 2). As can be seen in FIG. 10, all tested proteins mediated potent cellular phagocytosis by macrophages in a dose dependent manner.

The specification is most thoroughly understood in light of the teachings of the references cited within the specification. The aspects within the specification provide an illustration of aspects of the invention and should not be construed to limit the scope of the invention. The skilled artisan readily recognizes that many other aspects are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize or be able to ascertain using no more than routine experimentation, many equivalents to the specific aspects of the invention described herein. Such equivalents are intended to be encompassed by the following claims.

SEQUENCE TABLE

| SEQ | | |
|---|---|---|
| ID NO | Description | Sequence |
| 1 | Ankyrin repeat domain | DLGKKLLQAARAGQLDEVRELLKAGADVNAKDWSGFTPLHIAAVQGHLEIVEVLLKAGADVNAKDHSGWTPLHVAAFSGHLEI |
| | specific for CD16a | VEVLLKAGADVNAKDATGQTPLHLAARTGHLEIVEVLLKAGADVNAQDKSGKTPADLAARAGHQDIAEVLQKAA |
| 2 | Ankyrin repeat domain | DLGQKLLRAAKKGQDDEVRELLKAGADVNAKDEQGWTPLHIAAWKGHLEIVEVLLKAGADVNAKDVFDWTPLHVAAHFGHLE |
| | specific for CD16a | IVEVLLKAGADVNAQDRAGETPADLAAQAGHEDIAEVLQKAA |
| 3 | Ankyrin repeat domain | DLGKKLLQAARAGQLDEVRELLKAGADVNAKDEQGRTPLHIAAASGHLEIVEVLLKAGADVNAKDWFGITPLHIAAWWGHLEIV |
| | specific for CD16a | EVLLKAGADVNAKDQEGRTPLHIAAAIGHLEIVEVLLKAGADVNAQDKSGKTPADLAARAGHQDIAEVLQKAA |
| 4 | Ankyrin repeat domain | DLGEKLLLAAALGQDDEVRELLKAGADVNAKDVQGSTPLHLAAVEGHLEIVEVLLKAGADVNAKDQWGQTPLHVAAIWGHLEI |
| | specific for CD16a | VEVLLKAGADVNAQDELGKTPADLVALQGHEDIAEVLQKAA |
| 5 | Ankyrin repeat domain | DLGAKLLRAAKKGQDDEVRELLKAGADVNAKDELGFTPLHVAAEEGHLEIVEVLLKAGADVNAKDSWGATPLHVAAWVGHLEI |
| | specific for CD16a | VEVLLKAGADVNAQDQEGQTPADLAAEAGHEDIAEVLQKAA |
| 6 | Ankyrin repeat domain | DLGKKLLEAAEEGQDDEVRELLKAGADVNAKDEWGSTPLHIAAWVGHLEIVEVLLKAGADVNAKDSLGATPLHLAAEAGHLEI |
| | specific for CD16a | VEVLLKAGADVNAQDLKGKTPADLAALEGHEDIAEVLQKAA |
| 7 | Ankyrin repeat domain | DLGQKLLRAAKKGQDDEVRELLKAGADVNAKDEQGWTPLHNAAWKGHLEIVEVLLKAGADVNAKDVFDWTPLHVAAHFGHL |
| | specific for CD16a | EIVEVLLKAGADVNAQDRAGETPADLAAQAGHEDIAEVLQKAA |
| 8 | Ankyrin repeat domain | DLGKKLLEAARAGQDDEVRELLKAGADVNAKDEYGLTPLYLATAHGHLEIVEVLLKAGADVNAVDAIGFTPLHLAAFIGHLEIAE |
| | specific for HER2 | VLLKAGADVNAQDKFGKTPADIAAGAGNEDIAEVLQKAA |
| 9 | Ankyrin repeat domain | DLGIKLLFAAAKGQDDEVRELLKAGADVNAKDFQGVTPLHIAAQSGHLEIVEVLLKAGADVNAKDVTGDTPLHLAAQHGHLEIV |
| | specific for HER2 | EVLLKAGADVNAQDERGWTPADLAADWGHEDIAEVLQKAA |
| 10 | Ankyrin repeat protein | DLGKKLLEAARAGQDDEVRELLKAGADVNAKDEYGLTPLYLATAHGHLEIVEVLLKAGADVNAVDAIGFTPLHLAAFIGHLEIAE |
| | specific for CD16a and | VLLKAGADVNAQDKFGKTPADIAAGAGNEDIAEVLQKAAGSPTPTPTTPTPTPTTPTPTPTGSDLGQKLLRAAKKGQDDEVRE |
| | HER2 | LLKAGADVNAKDEQGWTPLHIAAWKGHLEIVEVLLKAGADVNAKDVFDWTPLHVAAHFGHLEIVEVLLKAGADVNAQDRAGET |
| | | PADLAAQAGHEDIAEVLQKAA |
| 11 | Ankyrin repeat protein | DLGIKLLFAAAKGQDDEVRELLKAGADVNAKDFQGVTPLHIAAQSGHLEIVEVLLKAGADVNAKDVTGDTPLHLAAQHGHLEIV |
| | specific for CD16a and | EVLLKAGADVNAQDERGWTPADLAADWGHEDIAEVLQKAAGSPTPTPTTPTPTPTTPTPTPTGSDLGQKLLRAAKKGQDDEV |
| | HER2 | RELLKAGADVNAKDEQGWTPLHIAAWKGHLEIVEVLLKAGADVNAKDVFDWTPLHVAAHFGHLEIVEVLLKAGADVNAQDRA |
| | | GETPADLAAQAGHEDIAEVLQKAA |
| 12 | Ankyrin repeat protein | DLGQKLLRAAKKGQDDEVRELLKAGADVNAKDEQGWTPLHIAAWKGHLEIVEVLLKAGADVNAKDVFDWTPLHVAAHFGHLE |
| | specific for CD16a and | IVEVLLKAGADVNAQDRAGETPADLAAQAGHEDIAEVLQKAAGSPTPTPTTPTPTPTTPTPTPTGSDLGKKLLEAARAGQDDE |
| | HER2 | VRELLKAGADVNAKDEYGLTPLYLATAHGHLEIVEVLLKAGADVNAVDAIGFTPLHLAAFIGHLEIAEVLLKAGADVNAQDKFGK |
| | | TPADIAAGAGNEDIAEVLQKAA |
| 13 | Ankyrin repeat protein | DLGQKLLRAAKKGQDDEVRELLKAGADVNAKDEQGWTPLHIAAWKGHLEIVEVLLKAGADVNAKDVFDWTPLHVAAHFGHLE |
| | specific for CD16a and | IVEVLLKAGADVNAQDRAGETPADLAAQAGHEDIAEVLQKAAGSPTPTPTTPTPTPTTPTPTPTGSDLGIKLLFAAAKGQDDEV |
| | HER2 | RELLKAGADVNAKDFQGVTPLHIAAQSGHLEIVEVLLKAGADVNAKDVTGDTPLHLAAQHGHLEIVEVLLKAGADVNAQDERG |
| | | WTPADLAADWGHEDIAEVLQKAA |
| 14 | Ankyrin repeat protein | DLGKKLLEAARAGQDDEVRELLKAGADVNAKDEYGLTPLYLATAHGHLEIVEVLLKAGADVNAVDAIGFTPLHLAAFIGHLEIAE |
| | specific for CD16a and | VLLKAGADVNAQDKFGKTPADIAAGAGNEDIAEVLQKAAGSPTPTPTTPTPTPTTPTPTPTGSDLGKKLLQAARAGQLDEVREL |
| | HER2 | LKAGADVNAKDEQGRTPLHIAAASGHLEIVEVLLKAGADVNAKDWFGITPLHIAAWWGHLEIVEVLLKAGADVNAKDQEGRTPL |
| | | HIAAAIGHLEIVEVLLKAGADVNAQDKSGKTPADLAARAGHQDIAEVLQKAA |
| 15 | Ankyrin repeat protein | DLGIKLLFAAAKGQDDEVRELLKAGADVNAKDFQGVTPLHIAAQSGHLEIVEVLLKAGADVNAKDVTGDTPLHLAAQHGHLEIV |
| | specific for CD16a and | EVLLKAGADVNAQDERGWTPADLAADWGHEDIAEVLQKAAGSPTPTPTTPTPTPTTPTPTPTGSDLGKKLLQAARAGQLDEV |
| | HER2 | RELLKAGADVNAKDEQGRTPLHIAAASGHLEIVEVLLKAGADVNAKDWFGITPLHIAAWWGHLEIVEVLLKAGADVNAKDQEG |
| | | RTPLHIAAAIGHLEIVEVLLKAGADVNAQDKSGKTPADLAARAGHQDIAEVLQKAA |
| 16 | Ankyrin repeat protein | DLGKKLLQAARAGQLDEVRELLKAGADVNAKDEQGRTPLHIAAASGHLEIVEVLLKAGADVNAKDWFGITPLHIAAWWGHLEIV |
| | specific for CD16a and | EVLLKAGADVNAKDQEGRTPLHIAAAIGHLEIVEVLLKAGADVNAQDKSGKTPADLAARAGHQDIAEVLQKAAGSPTPTPTTPT |
| | HER2 | PTPTTPTPTPTGSDLGKKLLEAARAGQDDEVRELLKAGADVNAKDEYGLTPLYLATAHGHLEIVEVLLKAGADVNAVDAIGFTP |
| | | LHLAAFIGHLEIAEVLLKAGADVNAQDKFGKTPADIAAGAGNEDIAEVLQKAA |
| 17 | Ankyrin repeat protein | DLGKKLLQAARAGQLDEVRELLKAGADVNAKDEQGRTPLHIAAASGHLEIVEVLLKAGADVNAKDWFGITPLHIAAWWGHLEIV |
| | specific for CD16a and | EVLLKAGADVNAKDQEGRTPLHIAAAIGHLEIVEVLLKAGADVNAQDKSGKTPADLAARAGHQDIAEVLQKAAGSPTPTPTTPT |
| | HER2 | PTPTTPTPTPTGSDLGIKLLFAAAKGQDDEVRELLKAGADVNAKDFQGVTPLHIAAQSGHLEIVEVLLKAGADVNAKDVTGDTP |
| | | LHLAAQHGHLEIVEVLLKAGADVNAQDERGWTPADLAADWGHEDIAEVLQKAA |
| 18 | Ankyrin repeat protein | DLGKKLLQAARAGQLDEVRELLKAGADVNAKDWSGFTPLHIAAVQGHLEIVEVLLKAGADVNAKDHSGWTPLHVAAFSGHLEI |
| | specific for CD16a and | VEVLLKAGADVNAKDATGQTPLHLAARTGHLEIVEVLLKAGADVNAQDKSGKTPADLAARAGHQDIAEVLQKAAGSPTPTPTT |
| | HER2 | PTPTPTTPTPTPTGSDLGIKLLFAAAKGQDDEVRELLKAGADVNAKDFQGVTPLHIAAQSGHLEIVEVLLKAGADVNAKDVTGD |
| | | TPLHLAAQHGHLEIVEVLLKAGADVNAQDERGWTPADLAADWGHEDIAEVLQKAA |
| 19 | Ankyrin repeat protein | DLGIKLLFAAAKGQDDEVRELLKAGADVNAKDFQGVTPLHIAAQSGHLEIVEVLLKAGADVNAKDVTGDTPLHLAAQHGHLEIV |
| | specific for CD16a and | EVLLKAGADVNAQDERGWTPADLAADWGHEDIAEVLQKAAGSPTPTPTTPTPTPTTPTPTPTGSDLGEKLLLAAALGQDDEV |
| | HER2 | RELLKAGADVNAKDVQGSTPLHLAAVEGHLEIVEVLLKAGADVNAKDQWGQTPLHVAAIWGHLEIVEVLLKAGADVNAQDELG |
| | | KTPADLVALQGHEDIAEVLQKAA |
| 20 | Ankyrin repeat protein | DLGIKLLFAAAKGQDDEVRELLKAGADVNAKDFQGVTPLHIAAQSGHLEIVEVLLKAGADVNAKDVTGDTPLHLAAQHGHLEIV |
| | specific for CD16a and | EVLLKAGADVNAQDERGWTPADLAADWGHEDIAEVLQKAAGSPTPTPTTPTPTPTTPTPTPTGSDLGAKLLRAAKKGQDDEV |
| | HER2 | RELLKAGADVNAKDELGFTPLHVAAEEGHLEIVEVLLKAGADVNAKDSWGATPLHVAAWVGHLEIVEVLLKAGADVNAQDQE |
| | | GQTPADLAAEAGHEDIAEVLQKAA |
| 21 | Ankyrin repeat protein | DLGIKLLFAAAKGQDDEVRELLKAGADVNAKDFQGVTPLHIAAQSGHLEIVEVLLKAGADVNAKDVTGDTPLHLAAQHGHLEIV |
| | specific for CD16a and | EVLLKAGADVNAQDERGWTPADLAADWGHEDIAEVLQKAAGSPTPTPTTPTPTPTTPTPTPTGSDLGKKLLEAAEEGQDDEV |
| | HER2 | RELLKAGADVNAKDEWGSTPLHIAAWVGHLEIVEVLLKAGADVNAKDSLGATPLHLAAEAGHLEIVEVLLKAGADVNAQDLKG |
| | | KTPADLAALEGHEDIAEVLQKAA |
| 22 | Ankyrin repeat protein | DLGKKLLEAARAGQDDEVRELLKAGADVNAKDYFSHTPLHLAARNGHLKIVEVLLKAGADVNAKDFAGKTPLHLAAADGHLEIV |
| | specific for CD16a and | EVLLKAGADVNAQDIFGKTPADIAADAGHEDIAEVLQKAAGSPTPTPTTPTPTPTTPTPTPTGSDLGQKLLRAAKKGQDDEVRE |
| | human serum albumin | LLKAGADVNAKDEQGWTPLHIAAWKGHLEIVEVLLKAGADVNAKDVFDWTPLHVAAHFGHLEIVEVLLKAGADVNAQDRAGET |
| | | PADLAAQAGHEDIAEVLQKAA |
| 23 | Ankyrin repeat protein | DLGKKLLEAARAGQDDEVRELLKAGADVNAKDYFSHTPLHLAARNGHLKIVEVLLKAGADVNAKDFAGKTPLHLAAADGHLEIV |
| | specific for CD16a and | EVLLKAGADVNAQDIFGKTPADIAADAGHEDIAEVLQKAAGSPTPTPTTPTPTPTTPTPTPTGSDLGKKLLQAARAGQLDEVRE |
| | human serum albumin | |
| | | LLKAGADVNAKDEQGRTPLHIAAASGHLEIVEVLLKAGADVNAKDWFGITPLHIAAWWGHLEIVEVLLKAGADVNAKDQEGRTP |
| | | LHIAAAIGHLEIVEVLLKAGADVNAQDKSGKTPADLAARAGHQDIAEVLQKAA |
| 24 | Ankyrin repeat domain | DLGKKLLEAARAGQDDEVRELLKAGADVNAKDYFSHTPLHLAARNGHLKIVEVLLKAGADVNAKDFAGKTPLHLAAADGHLEIV |
| | specific for human | EVLLKAGADVNAQDIFGKTPADIAADAGHEDIAEVLQKAA |
| | serum albumin | |
| 25 | Ankyrin repeat domain | DLGKKLLEAARAGQDDEVRELLKAGADVNAKDYFSHTPLHLAARNGHLKIVEVLLKAGADVNAKDFAGKTPLHLAANEGHLEIV |
| | specific for human | EVLLKAGADVNAQDIFGKTPADIAADAGHEDIAEVLQKAA |
| | serum albumin | |
| 26 | Ankyrin repeat domain | DLGKKLLEAARAGQDDEVRELLKAGADVNAKDYFSHTPLHLAARNGHLKIVEVLLKAGADVNAKDFAGKTPLHLAADAGHLEIV |
| | specific for human | EVLLKAGADVNAQDIFGKTPADIAADAGHEDIAEVLQKAA |
| | serum albumin | |
| 27 | Ankyrin repeat module | KDWSGFTPLHIAAVQGHLEIVEVLLKAGADVNA |
| 28 | Ankyrin repeat module | KDHSGWTPLHVAAFSGHLEIVEVLLKAGADVNA |
| 29 | Ankyrin repeat module | KDATGQTPLHLAARTGHLEIVEVLLKAGADVNA |
| 30 | Ankyrin repeat module | KDEQGWTPLHIAAWKGHLEIVEVLLKAGADVNA |
| 31 | Ankyrin repeat module | KDVFDWTPLHVAAHFGHLEIVEVLLKAGADVNA |
| 32 | Ankyrin repeat module | KDEQGRTPLHIAAASGHLEIVEVLLKAGADVNA |
| 33 | Ankyrin repeat module | KDWFGITPLHIAAWWGHLEIVEVLLKAGADVNA |
| 34 | Ankyrin repeat module | KDQEGRTPLHIAAAIGHLEIVEVLLKAGADVNA |
| 35 | Ankyrin repeat module | KDVQGSTPLHLAAVEGHLEIVEVLLKAGADVNA |
| 36 | Ankyrin repeat module | KDQWGQTPLHVAAIWGHLEIVEVLLKAGADVNA |
| 37 | Ankyrin repeat module | KDELGFTPLHVAAEEGHLEIVEVLLKAGADVNA |
| 38 | Ankyrin repeat module | KDSWGATPLHVAAWVGHLEIVEVLLKAGADVNA |
| 39 | Ankyrin repeat module | KDEWGSTPLHIAAWVGHLEIVEVLLKAGADVNA |
| 40 | Ankyrin repeat module | KDSLGATPLHLAAEAGHLEIVEVLLKAGADVNA |
| 41 | Ankyrin repeat module | KDEQGWTPLHNAAWKGHLEIVEVLLKAGADVNA |
| 42 | N-terminal capping | DLGKKLLQAARAGQLDEVRELLKAGADVNA |
| | module | |
| 43 | N-terminal capping | DLGQKLLRAAKKGQDDEVRELLKAGADVNA |
| | module | |
| 44 | N-terminal capping | DLGEKLLLAAALGQDDEVRELLKAGADVNA |
| | module | |
| 45 | N-terminal capping | DLGAKLLRAAKKGQDDEVRELLKAGADVNA |
| | module | |
| 46 | N-terminal capping | DLGKKLLEAAEEGQDDEVRELLKAGADVNA |
| | module | |
| 47 | C-terminal capping | QDKSGKTPADLAARAGHQDIAEVLQKAA |
| | module | |
| 48 | C-terminal capping | QDRAGETPADLAAQAGHEDIAEVLQKAA |
| | module | |
| 49 | C-terminal capping | QDELGKTPADLVALQGHEDIAEVLQKAA |
| | module | |
| 50 | C-terminal capping | QDQEGQTPADLAAEAGHEDIAEVLQKAA |
| | module | |
| 51 | C-terminal capping | QDLKGKTPADLAALEGHEDIAEVLQKAA |
| | module | |
| 52 | Ankyrin repeat module | KDXXGXTPLHXAAXXGHLEIVEVLLKAGADVNA |
| | | wherein X is selected from any amino acid. |
| 53 | Ankyrin repeat module | KDXXGXTPLHXAAXXGHPEIVEVLLKAGADVNA |
| | | wherein X is selected from any amino acid. |
| 54 | Ankyrin repeat module | XDXXGXTPLHXAAXXGHLEIVEVLLKXGADVNA |
| | | wherein X is selected from any amino acid. |
| 55 | Ankyrin repeat module | XDXXGXTPLHXAAXXGHPEIVEVLLKXGADVNA |
| | | wherein X is selected from any amino acid. |
| 56 | Ankyrin repeat module | XDXXGXTPLHXAAXXGHLEIVEVLLKAGADVNA |
| | | wherein X is selected from any amino acid. |
| 57 | Ankyrin repeat module | XDXXDXTPLHXAAXXGHLEIVEVLLKAGADVNA |
| | | wherein X is selected from any amino acid. |
| 58 | N-terminal capping | DLGXKLLXAAXXGQDDEVRELLKAGADVNA |
| | module | wherein X is selected from any amino acid. |
| 59 | N-terminal capping | DLGXKLLXAAXXGQLDEVRELLKAGADVNA |
| | module | wherein X is selected from any amino acid. |
| 60 | N-terminal capping | DLGXKLLEAAXXGQDDEVRELLKAGADVNA |
| | module | wherein X is selected from any amino acid. |
| 61 | N-terminal capping | DLGXKLLQAAXXGQLDEVRELLKAGADVNA |
| | module | wherein X is selected from any amino acid. |
| 62 | C-terminal capping | QDXXGXTPADLAAXXGHEDIAEVLQKAA |
| | module | wherein X is selected from any amino acid. |
| 63 | C-terminal capping | QDXXGXTPADLAAXXGHQDIAEVLQKAA |
| | module | wherein X is selected from any amino acid. |
| 64 | C-terminal capping | QDXXGXTPADLVAXXGHEDIAEVLQKAA |
| | module | wherein X is selected from any amino acid. |
| 65 | C-terminal capping | XDXXGXTPXXXXXDXXXEXXXXXXXXAA |
| | module | wherein X is selected from any amino acid. |
| 66 | C-terminal capping | XDXXGXTPXXXXXRXXXEXXXXXXXXAA |
| | module | wherein X is selected from any amino acid. |
| 67 | C-terminal capping | XDXXGXTPADLXAXXGHEDIAEVLQKAA |
| | module | wherein X is selected from any amino acid. |
| 68 | C-terminal capping | XDXXGXTPADLXAXXGHQDIAEVLQKAA |
| | module | wherein X is selected from any amino acid. |
| 69 | His Tag | MRGSHHHHHHGS |
| 70 | PT linker | GSPTPTPTTPTPTPTTPTPTPTGS |
| 71 | His-Flag Tag | MRGSHHHHHHDYKDDDDKPTPTPTGS |
| 72 | Target sequence | GMRTEDLPKAVVFLEPQWYRVLEKDSVTLKCQGAYSPEDNSTQWFHNESLISSQASSYFIDAATVDDSGEYRCQTNLSTLSD |
| | | PVQLEVHIGWLLLQAPRWVFKEEDPIHLRCHSWKNTALHKVTYLQNGKGRKYFHHNSDFYIPKATLKDSGSYFCRGLVGSKN |
| | | VSSETVNITITQGLAVSTISSFFPPGYQGGGSGGGSHHHHHHHHHH |
| 73 | Target sequence | MRTEDLPKAVVFLEPQWYRVLEKDSVTLKCQGAYSPEDNSTQWFHNENLISSQASSYFIDAATVDDSGEYRCQTNLSTLSDP |
| | | VQLEVHVGWLLLQAPRWVFKEEDPIHLRCHSWKNTALHKVTYLQNGKDRKYFHHNSDFHIPKATLKDSGSYFCRGLVGSKNV |
| | | SSETVNITITQGLAVSTISGGGSGGGSHHHHHHHHHH |
| 74 | Target sequence | GMRTEDLPKAVVFLEPQWYSVLEKDSVTLKCQGAYSPEDNSTQWFHNESLISSQASSYFIDAATVNDSGEYRCQTNLSTLSD |
| | | PVQLEVHIGWLLLQAPRWVFKEEDPIHLRCHSWKNTALHKVTYLQNGKDRKYFHHNSDFHIPKATLKDSGSYFCRGLVGSKN |
| | | VSSETVNITITQGLAVSTISGGGSGGGSHHHHHHHHHH |
| 75 | Target sequence | GMRTEDLPKAVVFLEPQWYRVLEKDSVTLKCQGAYSPEDNSTQWFHNESLISSQASSYFIDAATVDDSGEYRCQTNLSTLSD |
| | | PVQLEVHIGWLLLQAPRWVFKEEDPIHLRCHSWKNTALHKVTYLQNGKGRKYFHHNSDFYIPKATLKDSGSYFCRGLFGSKN |
| | | VSSETVNITITQGLAVSTISSFFPPGYQGLNDIFEAQKIEWHEGGGSGGGSHHHHHHHHHH |
| 76 | Target sequence | GMRAEDLPKAVVFLEPQWYRVLEKDRVTLKCQGAYSPEDNSTRWFHNESLISSQTSSYFIAAARVNNSGEYRCQTSLSTLSD |
| | | PVQLEVHIGWLLLQAPRWVFKEEESIHLRCHSWKNTLLHKVTYLQNGKGRKYFHQNSDFYIPKATLKDSGSYFCRGLIGSKNV |
| | | SSETVNITITQDLAVSSISSFFPPGYQGGGSGGGSHHHHHHHHHHGLNDIFEAQKIEWHE |
| 77 | Nucleic acid encoding | GACCTGGGTAAAAAACTGCTGCAAGCAGCACGTGCAGGTCAGCTGGATGAAGTTCGTGAACTGCTGAAAGCAGGCGCC |
| | domain of SEQ ID NO | GATGTTAATGCAAAAGATTGGTCTGGCTTCACCCCGCTGCATATCGCTGCTGTGCAAGGTCACCTGGAAATTGTTGAAGT |
| | 1 | TCTGCTGAAAGCCGGTGCAGATGTTAATGCAAAAGATCACTCTGGCTGGACCCCGCTGCATGTGGCTGCTTTCTCTGGT |
| | | CACCTGGAAATTGTTGAAGTTCTGCTGAAAGCCGGTGCAGATGTTAATGCAAAAGATGCTACCGGCCAAACCCCGCTGC |
| | | ATCTGGCTGCTAGAACCGGTCACCTGGAAATTGTTGAAGTTCTGCTGAAAGCCGGTGCAGATGTTAACGCACAGGATAAA |
| | | AGCGGTAAAACCCCTGCCGATCTGGCAGCTCGCGCCGGTCATCAAGATATTGCTGAAGTGCTGCAGAAGGCAGCA |
| 78 | Nucleic acid encoding | GACTTGGGACAAAAATTATTGCGCGCTGCAAAAAAAGGCCAGGACGACGAGGTGCGTGAGTTGCTTAAAGCAGGGGCC |
| | domain of SEQ ID NO | GATGTTAATGCAAAAGATGAACAAGGGTGGACGCCGTTGCATATTGCAGCGTGGAAAGGTCATTTAGAAATCGTAGAGGT |
| | 2 | GCTTCTTAAAGCGGGCGCTGACGTTAACGCAAAAGACGTATTCGATTGGACACCTTTGCATGTCGCTGCGCACTTTGGAC |
| | | ACTTAGAGATCGTGGAGGTTTTATTAAAGGCTGGCGCGGATGTAAATGCACAGGACCGTGCCGGAGAGACACCAGCGG |
| | | ATTTAGCAGCGCAAGCGGGACACGAAGACATCGCCGAGGTCCTGCAGAAGGCAGCG |
| 79 | Nucleic acid encoding | GACCTGGGTAAAAAACTGCTGCAAGCAGCACGTGCAGGTCAGCTGGATGAAGTTCGTGAACTGCTGAAAGCAGGCGCC |
| | domain of SEQ ID NO | GATGTTAATGCAAAAGATGAACAGGGCCGTACCCCGCTGCATATCGCTGCTGCTTCCGGTCACCTGGAAATTGTTGAAGT |
| | 3 | TCTGCTGAAAGCCGGTGCAGATGTTAATGCAAAAGATTGGTTCGGCATCACCCCGCTGCATATCGCTGCTTGGTGGGGT |
| | | CACCTGGAAATTGTTGAAGTTCTGCTGAAAGCCGGTGCAGATGTTAATGCAAAAGATCAGGAAGGCCGTACCCCGCTGC |
| | | ATATCGCTGCTGCTATCGGTCACCTGGAAATTGTTGAAGTTCTGCTGAAAGCCGGTGCAGATGTTAACGCACAGGATAAA |
| | | AGCGGTAAAACCCCTGCCGATCTGGCAGCTCGCGCCGGTCATCAAGATATTGCTGAAGTGCTGCAGAAGGCAGCG |
| 80 | Nucleic acid encoding | GACCTGGGTGAAAAGCTGCTGCTGGCTGCTGCTCTGGGTCAGGATGATGAAGTTCGTGAACTGCTGAAAGCAGGCGCC |
| | domain of SEQ ID NO | GATGTTAATGCAAAAGATGTGCAAGGCTCTACCCCGCTGCATCTGGCTGCTGTGGAGGGTCACCTAGAAATTGTTGAAGT |
| | 4 | TCTGCTGAAAGCCGGTGCAGATGTTAATGCAAAAGATCAATGGGGCCAAACCCCGCTGCATGTGGCTGCTATCTGGGGT |
| | | CACCTGGAAATTGTTGAAGTTCTGCTGAAAGCCGGTGCAGATGTTAACGCACAGGATGAACTGGGCAAGACCCCTGCCG |
| | | ATCTGGTAGCTCTGCAAGGTCATGAAGATATTGCAGAAGTGCTGCAGAAGGCAGCA |
| 81 | Nucleic acid encoding | GACCTGGGTGCTAAGCTGCTGCGTGCTGCTAAGAAGGGTCAGGATGATGAAGTTCGTGAACTGCTGAAAGCAGGCGCC |
| | domain of SEQ ID NO | GATGTTAATGCAAAAGATGAGCTGGGCTTCACCCCGCTGCATGTGGCTGCTGAGGAGGGTCACCTGGAAATTGTTGAAG |
| | 5 | TTCTGCTGAAAGCCGGTGCAGATGTTAATGCAAAAGATTCTTGGGGCGCTACCCCGCTGCATGTGGCTGCTTGGGTGGG |
| | | TCACCTGGAAATTGTTGAAGTTCTGCTGAAAGCCGGTGCAGATGTTAACGCACAGGATCAGGAAGGCCAGACCCCTGCC |
| | | GATCTGGCAGCTGAAGCTGGTCATGAAGATATTGCAGAAGTGCTGCAGAAGGCAGCA |
| 82 | Nucleic acid encoding | GACCTGGGTAAGAAGCTGCTGGAAGCTGCTGAAGAAGGTCAGGATGATGAAGTTCGTGAACTGCTGAAAGCAGGCGCC |
| | domain of SEQ ID NO | GATGTTAATGCAAAAGATGAGTGGGGCTCTACCCCGCTGCATATCGCTGCTTGGGTGGGTCACCTGGAAATTGTTGAAG |
| | 6 | TTCTGCTGAAAGCCGGTGCAGATGTTAATGCAAAAGATTCTCTGGGCGCTACCCCGCTGCATCTGGCTGCTGAGGCTGG |
| | | TCACCTGGAAATTGTTGAAGTTCTGCTGAAAGCCGGTGCAGATGTTAACGCACAGGATCTGAAGGGCAAGACCCCTGCC |
| | | GATCTGGCAGCTCTGGAAGGTCATGAAGATATTGCAGAAGTGCTGCAGAAGGCAGCA |
| 83 | Nucleic acid encoding | GGATCCGACTTGGGACAAAAATTATTGCGCGCTGCAAAAAAAGGCCAGGACGACGAGGTGCGTGAGTTGCTTAAAGCAG |
| | domain of SEQ ID NO | GGGCCGATGTTAATGCAAAAGATGAACAAGGGTGGACGCCGTTGCATAATGCAGCGTGGAAAGGTCATTTAGAAATCGT |
| | 7 | AGAGGTGCTTCTTAAAGCGGGCGCTGACGTTAACGCAAAAGACGTATTCGATTGGACACCTTTGCATGTCGCTGCGCAC |
| | | TTTGGACACTTAGAGATCGTGGAGGTTTTATTAAAGGCTGGCGCGGATGTAAATGCACAGGACCGTGCCGGAGAGACAC |
| | | CAGCGGATTTAGCAGCGCAAGCGGGACACGAAGACATCGCCGAGGTCCTGCAGAAGGCAGCG |
| 84 | Ankyrin repeat domain | DLGQKLLKAAQYGQDDEVRILLAAGADVNAKDWAGKTPLHVAAEAGHLEIVEVLLKAGADVNAKDKAGETPLHLAAEQGHLEI |
| | specific for CD117 | VEVLLKAGADVNAQDEWGQTPADLAAYYGLEDIAEVLQKAA |
| 85 | Ankyrin repeat domain | DLGAKLLKAAQSGQDDEVRILLAAGADVNAKDKDGYTPLHLAAREGHLEIVEVLLKAGSDVNAKDYYGETPLHQAAEYGHLEIV |
| | specific for CD117 | EVLLKAGADVNAQDEWGATPADLAAQQGHEDIAEVLQKAA |
| 86 | Ankyrin repeat domain | DLGKKLLQAARAGQLDEVRELLKAGADVNAKDVYGLTPLHLAAREGHLEIVEVLLKAGADVNAKDAYGETPLHLAAEAGHLEIV |
| | specific for CD117 | EVLLKAGADVNAKDEWGETPLNVAARAGHQDIAEVLQKAA |
| 87 | Ankyrin repeat domain | DLGKKLLKAASWGQDDEVRILLAAGADVNAKDNSGWTPLHAAAWYGHLEIVEVLLKAGADVNAKDKTGLTPLHKAAHVGHLEI |
| | specific for CD117 | VEVLLKAGADVNAQDEWGATPADLAASTGHEDIAEVLQKAA |
| 88 | Ankyrin repeat domain | DLGHKLLHAAAWGQDDEVRILLAAGADVNAKDQSGWTPLHVAAWYGHLEIVEVLLKAGADVNAKDLKGETPLHLAALVGHLEI |
| | specific for CD117 | VEVLLKAGADVNAQDIAGDTPADLAAIAGHEDIAEVLQKAA |
| 89 | Ankyrin repeat protein | DLGQKLLKAAQYGQDDEVRILLAAGADVNAKDWAGKTPLHVAAEAGHLEIVEVLLKAGADVNAKDKAGETPLHLAAEQGHLEI |
| | specific for CD16a and | VEVLLKAGADVNAQDEWGQTPADLAAYYGLEDIAEVLQKAAGSPTPTPTTPTPTPTTPTPTPTGSDLGQKLLRAAKKGQDDEV |
| | CD117 | RELLKAGADVNAKDEQGWTPLHIAAWKGHLEIVEVLLKAGADVNAKDVFDWTPLHVAAHFGHLEIVEVLLKAGADVNAQDRA |
| | | GETPADLAAQAGHEDIAEVLQKAA |
| 90 | Ankyrin repeat protein | DLGAKLLKAAQSGQDDEVRILLAAGADVNAKDKDGYTPLHLAAREGHLEIVEVLLKAGSDVNAKDYYGETPLHQAAEYGHLEIV |
| | specific for CD16a and | EVLLKAGADVNAQDEWGATPADLAAQQGHEDIAEVLQKAAGSPTPTPTTPTPTPTTPTPTPTGSDLGQKLLRAAKKGQDDEV |
| | CD117 | RELLKAGADVNAKDEQGWTPLHIAAWKGHLEIVEVLLKAGADVNAKDVFDWTPLHVAAHFGHLEIVEVLLKAGADVNAQDRA |
| | | GETPADLAAQAGHEDIAEVLQKAA |
| 91 | Ankyrin repeat protein | DLGKKLLQAARAGQLDEVRELLKAGADVNAKDVYGLTPLHLAAREGHLEIVEVLLKAGADVNAKDAYGETPLHLAAEAGHLEIV |
| | specific for CD16a and | EVLLKAGADVNAKDEWGETPLNVAARAGHQDIAEVLQKAAGSPTPTPTTPTPTPTTPTPTPTGSDLGQKLLRAAKKGQDDEV |
| | CD117 | RELLKAGADVNAKDEQGWTPLHIAAWKGHLEIVEVLLKAGADVNAKDVFDWTPLHVAAHFGHLEIVEVLLKAGADVNAQDRA |
| | | GETPADLAAQAGHEDIAEVLQKAA |
| 92 | Ankyrin repeat protein | DLGKKLLKAASWGQDDEVRILLAAGADVNAKDNSGWTPLHAAAWYGHLEIVEVLLKAGADVNAKDKTGLTPLHKAAHVGHLEI |
| | specific for CD16a and | VEVLLKAGADVNAQDEWGATPADLAASTGHEDIAEVLQKAAGSPTPTPTTPTPTPTTPTPTPTGSDLGQKLLRAAKKGQDDEV |
| | CD117 | RELLKAGADVNAKDEQGWTPLHIAAWKGHLEIVEVLLKAGADVNAKDVFDWTPLHVAAHFGHLEIVEVLLKAGADVNAQDRA |
| | | GETPADLAAQAGHEDIAEVLQKAA |
| 93 | Ankyrin repeat protein | DLGHKLLHAAAWGQDDEVRILLAAGADVNAKDQSGWTPLHVAAWYGHLEIVEVLLKAGADVNAKDLKGETPLHLAALVGHLEI |
| | specific for CD16a and | VEVLLKAGADVNAQDIAGDTPADLAAIAGHEDIAEVLQKAAGSPTPTPTTPTPTPTTPTPTPTGSDLGQKLLRAAKKGQDDEVR |
| | CD117 | ELLKAGADVNAKDEQGWTPLHIAAWKGHLEIVEVLLKAGADVNAKDVFDWTPLHVAAHFGHLEIVEVLLKAGADVNAQDRAGE |
| | | TPADLAAQAGHEDIAEVLQKAA |
| 94 | Ankyrin repeat protein | DLGQKLLKAAQYGQDDEVRILLAAGADVNAKDWAGKTPLHVAAEAGHLEIVEVLLKAGADVNAKDKAGETPLHLAAEQGHLEI |
| | specific for CD16a and | VEVLLKAGADVNAQDEWGQTPADLAAYYGLEDIAEVLQKAAGSPTPTPTTPTPTPTTPTPTPTGSDLGQKLLRAAKKGQDDEV |
| | CD117 | RELLKAGADVNAKDEQGWTPLHNAAWKGHLEIVEVLLKAGADVNAKDVFDWTPLHVAAHFGHLEIVEVLLKAGADVNAQDRA |
| | | GETPADLAAQAGHEDIAEVLQKAA |
| 95 | Control Construct 1 | DLGKKLLEAARAGQDDEVRELLKAGADVNAKDKDGYTPLHLAAREGHLEIVEVLLKAGADVNAKDKDGYTPLHLAAREGHLEI |
| | | VEVLLKAGADVNAQDKSGKTPADLAADAGHEDIAEVLQKAAGSPTPTPTTPTPTPTTPTPTPTGSDLGQKLLRAAKKGQDDEV |
| | | RELLKAGADVNAKDEQGWTPLHIAAWKGHLEIVEVLLKAGADVNAKDVFDWTPLHVAAHFGHLEIVEVLLKAGADVNAQDRA |
| | | GETPADLAAQAGHEDIAEVLQKAA |
| 96 | Control Construct 2 | DLGKKLLEAARAGQDDEVRELLKAGADVNAKDKDGYTPLHLAAREGHLEIVEVLLKAGADVNAKDKDGYTPLHLAAREGHLEI |
| | | VEVLLKAGADVNAQDKSGKTPADLAADAGHEDIAEVLQKAAGSPTPTPTTPTPTPTTPTPTPTGSDLGQKLLRAAKKGQDDEV |
| | | RELLKAGADVNAKDEQGWTPLHNAAWKGHLEIVEVLLKAGADVNAKDVFDWTPLHVAAHFGHLEIVEVLLKAGADVNAQDRA |
| | | GETPADLAAQAGHEDIAEVLQKAA |
| 97 | Ankyrin repeat module | KDXXDXTPLHXAAXXGHLEIVEVLLKAGADVNA |
| | | wherein X is selected from any amino acid. |

1-17. (canceled)

18. A recombinant binding protein, comprising an ankyrin repeat domain having binding specificity for CD16a, wherein the ankyrin repeat domain comprises an ankyrin repeat module having an amino acid sequence selected from:
(i) SEQ ID NOs: 27 to 41, and
(ii) sequences having up to 9 amino acids substitutions by other amino acids, relative to SEQ ID NOs: 27 to 41.

19. The recombinant binding protein according to claim 18, wherein the ankyrin repeat module is a first ankyrin repeat module, and wherein the ankyrin repeat domain further comprises a second ankyrin repeat module having an amino acid sequence selected from:
(i) SEQ ID NOs: 27 to 41, and
(ii) sequences having up to 9 amino acids substituted by other amino acids, relative to any of SEQ ID NOs: 27 to 41.

20. The recombinant binding protein according to claim 19, wherein the first and second ankyrin repeat modules each independently comprise an amino acid sequence selected from:
(i) SEQ ID NO: 30, 31, 35, 36 and 37 to 41, and
(ii) sequences having up to 9 amino acids substituted by other amino acids, relative to any of SEQ ID NO: 30, 31, 35, 36 and 37 to 41.

21. The recombinant binding protein according to claim 19, wherein the first ankyrin repeat module comprises an amino acid sequence selected from:
(i) SEQ ID NO: 30, and
(ii) sequences having up to 9 amino acids substituted by other amino acids, relative to SEQ ID NO: 30,
wherein the second ankyrin repeat module comprises an amino acid sequence selected from:
(i) SEQ ID NO: 31, and
(i) sequences having up to 9 amino acid substituted by other amino acids, relative to of SEQ ID NO: 31.

22. The recombinant binding protein according to claim 19, wherein the ankyrin repeat domain further comprises a third ankyrin repeat module having an amino acid sequence selected from:
(i) SEQ ID NOs: 27 to 41, and
(ii) sequences having up to 9 amino acids substituted by other amino acids, relative to any of SEQ ID NOs: 27 to 41.

23. The recombinant binding protein according to claim 22, wherein the first, second and third ankyrin repeat modules each independently comprise an amino acid sequence selected from:
(i) SEQ ID NO: 27 to 29 and 32 to 34, and
(ii) sequences having up to 9 amino acids substituted by other amino acids, relative to any of SEQ ID NO: 27 to 29 and 32 to 34.

24. The recombinant binding protein according to claim 19, wherein the first ankyrin repeat module is located N-terminally of said second ankyrin repeat module within the ankyrin repeat domain.

25. The recombinant binding protein according claim 22, wherein the first ankyrin repeat module is located N-terminally of the second ankyrin repeat module, and wherein the second ankyrin repeat module is located N-terminally of the third ankyrin repeat module within the ankyrin repeat domain.

26. A recombinant binding protein, comprising an ankyrin repeat domain with binding specificity for CD16a, wherein the ankyrin repeat domain comprises an amino acid sequence with at least 80% amino acid sequence identity with any one of SEQ ID NOs: 1 to 7.

27. The recombinant binding protein according to claim 26, wherein:
(a) the alanine in the penultimate position of SEQ ID NOs: 1 to 7 is substituted by leucine, or
(b) the alanine at the last position of SEQ ID NOs: 1 to 7 is substituted by asparagine, or
(c) both (a) and (b).

28. The recombinant binding protein according to claim 26, wherein the ankyrin repeat domain comprises an amino acid sequence with at least 80% amino acid sequence identity with SEQ ID NO: 2, and wherein the alanine at the penultimate position of SEQ ID NO: 2 is optionally substituted by leucine, and/or the alanine at the last position of SEQ ID NO: 2 is optionally substituted by asparagine.

29. The recombinant binding protein according to claim 18, wherein the binding protein binds human CD16a in PBS with a dissociation constant (KD) below 10⁻⁷ M.

30. The recombinant binding protein according to claim 18, wherein the binding protein does not substantially bind human CD16b.

31. The recombinant binding protein according to claim 18, wherein the recombinant binding protein further comprises one, two or three binding moieties, wherein each of the binding moieties has binding specificity for a disease-associated antigen.

32. The recombinant binding protein according to claim 31, wherein at least one of the binding moieties with binding specificity for a disease-associated antigen is an ankyrin repeat domain.

33. The recombinant binding protein according to claim 18, wherein the recombinant binding protein further comprises a half-life extending moiety.

34. A nucleic acid, encoding the binding protein according to claim 18.

35. A pharmaceutical composition, comprising the binding protein according to claim 18 and a pharmaceutically acceptable carrier and/or diluent.

36. A method of treating a medical condition, comprising the step of administering to a subject in need thereof a therapeutically effective amount of the recombinant binding protein according to claim 18.

37. The method according to claim 36, wherein said medical condition is cancer.
